(19) United States
(12) Patent Application Publication
(10) Pub. No.: US 2013/0316326 A1
(43) Pub. Date: Nov. 28, 2013

(54) ANIMAL MODEL FOR THE EVALUATION OF THE EFFICACY OF AN HIV VACCINE

(71) Applicant: Elena Yu Filinova, Moscow (RU)

(72) Inventor: Elena Yu Filinova, Moscow (RU)

(73) Assignee: TECHNOLOGIE INTEGRALE LTD., Ramsey (GB)

(21) Appl. No.: 13/818,890

(22) PCT Filed: Jun. 3, 2011
(86) PCT No.: PCT/EP2011/002737

§ 371 Date: Jun. 25, 2013

(30) Foreign Application Priority Data
Aug. 27, 2010 (EP) 10008952.3

(51) Int. Cl. A01K 67/027; A61K 39/21; G01N 33/569

(52) U.S. Cl. CPC A01K 67/0271; G01N 33/56988; A61K 39/21
USPC 435/5; 506/9; 424/208.1; 424/188.1; 536/23.72; 800/11

(57) ABSTRACT

The present invention relates to the use of a Severe Combined T-B-Immune Deficient (SCID) mouse engrafted with human immunocompetent cells (Hu-SCID-mouse) as an animal model for the evaluation of the effectiveness of an HIV vaccine. Furthermore, the present invention relates to a method for the evaluation of an HIV vaccine, wherein a Hu-SCID-mouse of the invention is inoculated with the HIV vaccine and thereafter challenged with HI-virus. The invention also relates to novel HIV vaccine compositions, which can be evaluated using the animal model.

The present application relates to the new attitude of an HIV preventive vaccine development, the particular details of its production and composition description and the method of the evaluation of the efficacy of an HIV vaccine in the laboratory animal model—sever combined T-B-immune deficient mice (SCID mice). Moreover, the present invention relates to an animal model for the evaluation of the efficacy of an HIV vaccine.

Library approach and mass spectrometry quantitative HIV envelop proteins variability analysis as well as in vivo confirmation of a correlation between HIV gp120-gp160 infection-active envelop protein complexes presentation with variability of human cells CD4 and co-receptors responsible for HIV infection obtaining and development in humans are in the background of the present invention.

The current application describes the method of immunizations and challenging of the immune deficient SCID mice capable for engraftment with human immunocompetent cells (Hu-SCID-mouse) as an animal model for the evaluation of the effectiveness of an HIV vaccine.

### SUMMARY OF THE INVENTION

The very first mathematic modeling calculations and mass spectrometry analysis of the variability of HIV envelop proteins collected using antibodies libraries and reverse palming techniques showed a huge gap between possible sequence variability up to 10⁶ for a single patient and a number of gp120 major representatives, responsible for HIV infection catching and further AIDS disease progression. The difference in these “calculated” and “found out” sequence numbers was similar to an in vitro disparity between HIV RNA titre measured in copies per ml in culture media and tissue cell infectivity dose (TCID₅₀) which is usually 10⁶-10⁸ lower than HIV RNA titre for laboratory strains.

A simple explanation of these differences was found. The number of gp120 sequence variability and/or a number gp160 trimer isoforms which provides infectiously active HI-virus particles survival selection and advantages for further multiplication depends on the number of human cells receptors such as CD4, CCR5 and CXCR4 variations available for virus invasion. As the variability of human peripheral blood mononuclear (PBMC) and other cells membrane protein's sequences and isoforms exists but is limited by eukaryotic genome stability the number of passed cell-invading selection HIV gp160 infectiously-capable envelop proteins variations is limited to several decades or one-two hundred variants maximum too.

The assays of this theory were confirmed in HIV-1 laboratory strain's and their “playbacks”—the same laboratory strains human PBMC in vitro cultivation isolate's vaccine compositions. Results were obtained as in vivo immunizations efficiency evaluation in SCID mice engrafted with human immunocompetent cells (PBMC, DC) and challenged with HIV laboratory strain and their “playback” PBMC in vitro derivatives according to protocols described in detailed description of the invention.

In a preferred embodiment, the HIV vaccine is an immunogenic composition consist of HIV-1 envelop peptides/proteins cocktail in the presence of an adjuvant, wherein the proteins or peptides are of recombinant polyclonal origin and which immune boost adjuvant enhances vaccine efficiency through subcutaneous inoculation.

In another aspect, the invention relates to a method for the evaluation of an HIV vaccine, wherein a hu-SCID-mouse is inoculated with the HIV vaccine and thereafter challenged with HI-virus, wherein the efficacy is determined by determining the protection to HIV challenge.

Animal models for studying HIV infection were known in the art. These models include monkeys, and also SCID mice optionally engrafted with human leukocytes. However, in the past, the animal models used for studying preclinical assays of HIV vaccine forms were run only in a very limited number of animal models, mostly in two models only—Macaque Rhesus and Chimpanzees. In macaque rhesus human HIV infection does not work, so vaccines were tested exclusively for eliciting HIV-specific antibodies response with extremely sensitive ELISPOT kit's assays. In chimps that are bearing HIV infection but without developing any symptoms of immune deficiency disease (AIDS) finally tests results were resumed to the same ELISPOT data.

Therefore, there is a need for an efficient animal model for testing HIV vaccines, and for novel, efficient HIV vaccine compositions.

In one aspect, the present invention relates to the use of a Severe Combined T-B-Immune Deficient (SCID) mouse engrafted with human immunocompetent cells (Hu-SCID-mouse) as an animal model for the evaluation of the effectiveness of an HIV vaccine.

In a preferred embodiment, the immunocompetent cells are able to develop a human-type immune reaction for HIV. Preferably, the immune reaction is detectable in the blood of the animal, in particular in blood serum.

In a further preferred embodiment, the human immunocompetent cells are PBMC, dendritic cells or a mixture of PBMC and dendritic cells.

In a preferred embodiment, the human immunocompetent cells are PBMC, in particular PBMC pre-cultivated in vitro, more particularly PBMC pre-cultivated in vitro for a short period of time, even more preferred for about 1 day to about 6 weeks.

In a further preferred embodiment, the dendritic cells have been obtained by culturing human PBMC in the presence of cytokines capable of inducing the formation of dendritic cells in vitro. Preferably, the cytokines are also capable of cell differentiation blockage.

In a preferred embodiment, the human immunocompetent cells are derived from one human donor.

In a preferred embodiment, the mouse has been engrafted with 5-15×10⁶ cells, in particular immunocompetent cells.

In a preferred embodiment, the Hu-SCID mouse is the particular breed of animals deficient in their own endogenous immune system and introduced with human immunocompetent cells or any other human cells, in particular human immunocompetent cells.

In a preferred embodiment, the Hu-SCID mouse is the particular breed of animals deficient in their own endogenous immune system and can bear the engraftment of human immunocompetent cells, PBMC, dendrite cells (DC) or a mixture of PBMC and dendrite cells able to develop a human-type immune reaction for HIV.

In another aspect, the invention relates to a method for the evaluation of an HIV vaccine, wherein a Hu-SCID-mouse of the invention is inoculated with the HIV vaccine and thereafter challenged with HI-virus.

In a preferred embodiment, the HIV vaccine is an HIV-1 envelop peptides/proteins cocktail, preferably wherein the proteins or peptides are of recombinant origin.

In a preferred embodiment, the evaluation of the vaccine is determined by determining the efficacy of the vaccine.

In a preferred embodiment, the efficacy is determined by determining the protection to HIV challenge.

In a preferred embodiment, the Hu-SCID-mouse is inoculated with the HIV vaccine 1 to 4 weeks after the last engrafting of the human immunocompetent cells.

In a preferred embodiment, the human immunocompetent cells, PBMC or DC, OR A MIXTURE of PBMC and dendrite cells, intended for one mouse for one experiment are delivered from one human donor and have been engrafted as 5-15×10⁶ cell for one inoculation for a single animal.

An infectious doses of 5×10²-10⁴ TCID₅₀ or higher, preferably higher than 10⁴ TCID₅₀ for PBMC-cultivated and laboratory HIV strains are used for challenging of one Hu-SCID animal.

Preferably, infectious doses of at least 10⁴ TCID₅₀, more preferably of at least 10⁵ TCID₅₀, even more preferred of at least 10⁶ TCID₅₀ are used. Preferably, infectious doses of up to 10⁷ TCID₅₀ are used.

In a preferred embodiment, the efficacy of the HIV vaccine is determined by detection of the viral load, in particular the absence/presence of the viral load, and/or the specificity an/or the intensity of an immune response in a body fluid sample of the mouse. In particular, the efficacy of the HIV vaccine is determined by detection of the viral load absence/presence and/or the specificity of an immune response in a body fluid sample of the mouse.

In a preferred embodiment, the body fluid sample is blood serum. As HI virus infection in humans is being detected in blood samples the other tests like intraperitoneal liquid/cells viral load PCR assays or ELISA immune response assays can be due to residue ex vivo effect and should not be taken as vaccine efficiency results.

In a preferred embodiment, the inoculation with a vaccine is performed in the presence of an adjuvant.

The acceptability of control Hu-SCID mice to HIV challenging as well as their resistance to the challenging after vaccinations are preferably detected as viral load in the bloodstream measured as a number of HIV RNA copies in 1 ml serum by means of Real Time PCR.

The dynamics of HIV laboratory strains and PBMC-DC-“playback” strains replication in vitro controls is preferably carried out by means of Real Time PCR and p24 ELISA.

The HIV specificity of an immune response in a body fluid sample of the mouse is preferably carried out by means of ELISA with envelop p120-p160 antigens.

In a preferred embodiment, HIV-specific immune response for PBMC-Hu-SCID or DC-PBMC-Hu-SCID mice is detectable within the period of several weeks after the last vaccination.

In a preferred embodiment, the evaluation of the vaccine is carried out by means of RT-PCR, Real Time PCR or ELISA.

In a preferred embodiment, at least one HIV laboratory strain playback is used to infect and/or expose the Hu-SCID mice with/to HIV.

A method for producing an HIV vaccine, is described in WO2009/046984. Regarding the description and disclosure of the method and method steps, it is explicitly referred to the respective sections in WO2009/046984.

In particular the present invention also relates to a method for producing an HIV vaccine and are recombinant proteins/peptides cocktail composition obtained thereby, wherein the method comprises the steps:
a) creation of a library comprising HIV-1 specific antibodies,
b) enrichment for HIV-1-specific antibodies in the library by panning with HIV-1 peptides, in particular native and/or recombinant HIV-1 peptides,
c) multiplying HIV-1 material comprising HIV-1 peptides, polypeptides or proteins,
d) collecting HIV-1 peptides of the multiplied HIV-1 material using HIV-1-specific antibodies of step b) bound to a support,
e) identification and characterization of the HIV-1 peptides obtained in step d) by mass spectrometry, in particular by MS-MS,
f) cloning of fragments of the gp120 gp160 genes encoding the peptides identified in step e),
g) expressing glycosylated env HIV-1 peptides using the results of step f) in a eukaryotic expression system,
h) purification of the glycosylated env HIV-1 peptides, and
i) production of a vaccine composition,
   characterized in that for cloning and/or expressing glycosylated env HIV-1 peptides in step f) and/or g), at least one primer selected from the group consisting of:
(i) V1 forward for subtypes A, G B, C, F1, and H:

(SEQ ID No. 1)

5′-CTC TGY GTY ACT TTA XXX XXX XXX-3′,

(ii) V2 forward for all subtypes:

(SEQ ID No. 3)

5′-AAA ACT GCT CTT WCA XXX XXX XXX-3′,

(iii) V3 forward for A, B, G subtypes:

(SEQ ID No. 5)

5′-TAV AAA TTA ATT GTA XXX XXX XXX X-3′,

(iv) V3 forward for subtype D:

(SEQ ID No. 7)

5′-TAV CAA TTA ATT GCA XXX XXX XXX X-3′,

(v) V4 forward for all subtypes:

(SEQ ID No. 9)

5′-GAA TTT TTC TAT TGY AAXXX XXX XXX-3′,

(vi) V5 forward for A, B, D, G subtypes:

(SEQ ID No. 11)

5′-ACA AGA GAT GGT GGX XXX XXX X-3′,
and

(vii) V5 forward for subtype C:

(SEQ ID No. 13)

5′-ACA CGT GAT GGA GGX XXX XXX X-3′,

And at least one primer selected from the group consisting of:
(viii) gp41(160) Reverse for A, B, D subtypes, including sequence encoding His-Tag:

(SEQ ID No. 46)
ATA GCG GCC GCC TAG TGG TGG TGA TGG TGG TGT AG YAA
AGC YCT TTC NAA GCC CTG TC,

(ix) a primer derivative of (viii) lacking the sequence encoding a His-Tag, and/or comprising a sequence encoding a tag,
(x) gp41(160) Reverse for subtype A, rare variant:

(SEQ ID No. 66)
ATA GCG GCC GCC TAG TGG TGG TGA TGG TGG TGT AGC
AAA GCY CTT TCN GCG CCC TGT C,

(xi) gp41(160) Reverse for C subtype:

(SEQ ID No. 67)
ATA GCG GCC GCC TAG TGG TGG TGA TGG TGG TGT WGC
AAA GCT GCT TCA AAG CCC TGT C,

(xii) gp41(160) Reverse for G subtype:

(SEQ ID No. 68)
ATA GCG GCC GCC TAG TGG TGG TGA TGG TGG TGT AGC
AAA GCY CTT TCN AAG CCT TGT C,

(xiii) gp120 Reverse Const5 for all subtypes, including sequence encoding His-Tag:

(SEQ ID No. 52)
5′-ATA GCG GCC GCC TAG TGG TGG TGA TGG TGG TGT CTT
TTT TCT CTY TSC ACC ACT CTY CT-3′,

and
(xiiii) a primer derivative of (viii) lacking the sequence encoding a His-Tag, and/or comprising a sequence encoding a tag,
   are used.

In a preferred embodiment, the primers used are suitable for subtype identification. For example primer (xi) gp41(160) Reverse for C subtype and primer (vii) V5 forward for subtype C may be used for subtype C identification.

In a preferred embodiment, 2, 3, 4, 5, 6, 7, 8, 9, 10, 12, 15, 18 or more variants of primer (i), (ii), (iii), (iv), (v), (vi) and/or (vii) are used, wherein the variants differ in the variables “X”.

In a preferred embodiment, the primers (i) to (vii), and (x) to (xii) and one of (xiii) and (xiiii) and one of (viii) and (ix) are used.

In a further embodiment, the present invention relates to a method for producing an HIV vaccine composition, comprising the steps of:
a) creation of a library comprising HIV-1 specific antibodies,
b) enrichment for HIV-1-specific antibodies in the library by panning with HIV-1 peptides, in particular native and/or recombinant HIV-1 peptides,
c) multiplying HIV-1 material comprising HIV-1 peptides, polypeptides or proteins,
d) collecting HIV-1 peptides of the multiplied HIV-1 material using HIV-1-specific antibodies of step b) bound to a support,
e) identification and characterization of the HIV-1 peptides obtained in step d) by mass spectrometry, in particular by MS-MS,
f) cloning of fragments of the gp120 gp160 genes encoding the peptides identified in step e),
g) expressing glycosylated env HIV-1 peptides using the results of step f) in a eukaryotic expression system,
h) purification of the glycosylated env HIV-1 peptides, and
i) production of a vaccine composition,
   characterized in that for cloning and/or expressing glycosylated env HIV-1 peptides in step 1) and/or g), at least one primer selected from the group consisting of:
(i) Forward gp120 Const1 for A subtype (with XbaI site on 5′ end):
   5′-AAT TCT AGA CRC TRC AGA AAA CTT GTG GGT YAC-3′ (SEQ ID No. 52), or a derivative thereof lacking the XbaI site, and/or comprising a restriction site at the 5′ end,
(ii) Forward gp120 Const1 for B subtype (with XbaI site on 5′ end):
   5′-AAT TCT AGA CGC TRC AGA AMA ATT GTG GGT CAC-3′ (SEQ ID No. 69), or a derivative thereof lacking the XbaI site, and/or comprising a restriction site at the 5′ end,
(iii) Forward gp120 Const1 for C subtype (with XbaI site on 5′ end):
   5′-AAT TCT AGA CGT RRT GGG RAA CTT GTG GGT CAC-3′ (SEQ ID No. 70), or a derivative thereof lacking the XbaI site, and/or comprising a restriction site at the 5′ end, and
(iv) Forward gp120 Const1 for G subtype (with XbaI site on 5′ end):
   5′-AAT TCT AGA CGC CTC ARA TAA CTT GTG GGT CAC AG-3′(SEQ ID No. 71), or a derivative thereof lacking the XbaI site, and/or comprising a restriction site at the 5′ end,
      And at least one primer selected from the group consisting of:
(v) V1 reverse for all subtypes:

(SEQ ID No. 25)

5′-GCA GTT TTT YAT TTC TYX XXX XXX XXX-3′,

(vi) V2 reverse for subtypes A, D, C:

(SEQ ID No. 28)

5′-AGG TAT TRC AAT TTA TTX XXX XXX X-3′,

(vii) V2 reverse for subtype B:

(SEQ ID No. 29)

5′-CTG AGG TRT TAC AAX XXX XXX X-3′,

(viii) V2 reverse for subtype G:

(SEQ ID No. 30)

5′-AGA CAT TAC AAT TTA TTX XXX XXX X-3′,

(ix) V2 reverse for subtype F1:

(SEQ ID No. 31)

5′-TTG AGG TAT TRC AAX XXX XXX X-3′,

(x) V3 reverse for subtypes A, D, (C):

(SEQ ID No. 40)

5′-AAA GTT TBA TTC CAX XXX XXX XX-3′,

(xi) V3 reverse for subtype B:

(SEQ ID No. 41)

5′-AAA GTG TTR TTC CAX XXX XXX XX-3′,

(xii) V4 reverse for subtype G:

(SEQ ID No. 19)

5′-CAA TTT GTT TTA TYY TAC A XX XXX XXX X-3′,

(xiii) V4 reverse for subtypes A, B, C, D:

(SEQ ID No. 20)

5′-TAA TTT GYT TTA TTY TGC A XX XXX XXX X-3′,

(xiv) V5 reverse for all subtypes:

(SEQ ID No. 15)

5′-TCC TCC TSC AGG TCT GAA XXX XXX XXX X-3′,

(xv) gp41(160) Reverse for A, B, D subtypes, including sequence encoding His-Tag:

(SEQ ID No. 46)
ATA GCG GCC GCC TAG TGG TGG TGA TGG TGG TGT AG YAA
AGC YCT TTC NAA GCC CTG TC,

(xvi) a primer derivative of (viii) lacking the sequence encoding a His-Tag, and/or comprising a sequence encoding a tag,
(xvii) gp41(160) Reverse for subtype A, rare variant:

(SEQ ID No. 66)
ATA GCG GCC GCC TAG TGG TGG TGA TGG TGG TGT AGC
AAA GCY CTT TCN GCG CCC TGT C,

(xviii) gp41(160) Reverse for C subtype:

(SEQ ID No. 67)
ATA GCG GCC GCC TAG TGG TGG TGA TGG TGG TGT WGC
AAA GCT GCT TCA AAG CCC TGT C,

and
(xix) gp41(160) Reverse for G subtype:

(SEQ ID No. 68)

ATA GCG GCC GCC TAG TGG TGG TGA TGG TGG TGT AGC
AAA GCY CTT TCN AAG CCT TGT C,

are used.

In a preferred embodiment, the primers used are suitable for subtype identification. For example primer (i) Forward gp120 Const1 for A subtype and primer (vi) V2 reverse for subtypes A, D, C may be used for subtype A identification.

In a preferred embodiment, 2, 3, 4, 5, 6, 7, 8, 9, 10, 12, 15, 18 or more variants of primer (v), (vi), (vii), (viii), (ix), (x), (xi), (xii), (xiii) and/or (xiv) are used, wherein the variants differ in the variables, in particular the variables “X”.

In a preferred embodiment, the primers (i) to (xiv), and (xvii) to (xix) and one of (xv) and (xvi) are used.

According to the present invention, “X” in the nucleotide sequences is understood as, independently from each other, a DNA nucleotide selected from A, C, T and G.

In a preferred embodiment, the library is a human antibodies library.

In a further preferred embodiment, the library is a phagemid library or native antibodies library, or HIV envelop-specific peptides/proteins library bound to a solid support.

The antibodies may be in IgG, in particular an IgG1, IgG2, IgG3, IgG4, or IgM, IgA1, IgA2, IgAsec, IgD or IgE format. In a further preferred embodiment, the antibodies of the library are in IgGformat.

In a further preferred embodiment scFv antibodies, in particular scFv antibodies in a phagemid library may be used, as shown in the examples.

HIV envelop-specific peptides/proteins library may be presented as proteins and/or peptides selected for HIV specificity and capable to bind HIV gp120-gp160 proteins in enzyme-immune reactions, flow cytometry, fluorescent microscopy or Western blotting tests. Such envelop-specific peptides/proteins may be based on scaffolds like the DARPIN or lipocalin scaffold.

According to the present invention, “antibody” is understood as encompassing antibodies, antibody fragments and antibody mimetics. Antibody fragments encompass for example Fab, F(ab′)2, scFv (single chain Fv), diabodies, single domain VHH, VH or VL single domains. A particularly preferred antibody is the scFv antibody fragment. Antibody mimetics encompass for example DARPINs and Lipocalins.

In a preferred embodiment, the sequence variables “X” in the primers are specified by
(a) identifying the peptide sequences or parts thereof according to method step (e) above, and
(b) identifying the nucleotide sequences encoding the peptide sequences or parts thereof of step (a).

In particular, step (b) is performed by taking into account the standard triplet code. This is for example described in Example 4.1.

In a further embodiment, the present invention relates to a composition comprising at least one of the following nucleic acids:
(i) V1 forward for subtypes A, G, B, C, F1, H:

(SEQ ID No. 1)

5′-CTC TGY GTY ACT TTA XXX XXX XXX-3′,

(ii) V2 forward for all subtypes:

(SEQ ID No. 3)

5′-AAA ACT GCT CTT WCA XXX XXX XXX-3′,

(iii) V3 forward for A, B, G subtypes:

(SEQ ID No. 5)

5′-TAV AAA TTA ATT GTA XXX XXX XXX X-3′,

(iv) V3 forward For subtype D:

(SEQ ID No. 7)

5′-TAV CAA TTA ATT GCA XXX XXX XXX X-3′,

(v) V4 forward for all subtypes:

(SEQ ID No. 9)

5′-GAA TTT TTC TAT TGY AAXXX XXX XXX-3′,

(vi) V5 forward for A, B, D, G subtypes:

(SEQ ID No. 11)

5′-ACA AGA GAT GGT GGX XXX XXX X-3′,

(vii) V5 forward for subtype C:

(SEQ ID No. 13)

5′-ACA CGT GAT GGA GGX XXX XXX X-3′,

(viii) gp41(160) Reverse for A, B, D subtypes, including sequence encoding His-Tag:
   ATA GCG GCC GCC TAG TGG TGG TGA TGG TGG TGT AG YAA AGC YCT TTC NAA GCC CTG TC (SEQ ID No. 46) or a derivative thereof lacking the sequence encoding a His-Tag, and/or comprising a sequence encoding a tag,
(ix) gp41(160) Reverse for subtype A, rare variant:

(SEQ ID No. 66)

ATA GCG GCC GCC TAG TGG TGG TGA TGG TGG TGT AGC
AAA GCY CTT TCN GCG CCC TGT C,

(x) gp41(160) Reverse for C subtype:

(SEQ ID No. 67)

ATA GCG GCC GCC TAG TGG TGG TGA TGG TGG TGT WGC
AAA GCT GCT TCA AAG CCC TGT C,

(xi) gp41(160) Reverse for G subtype:

(SEQ ID No. 68)

ATA GCG GCC GCC TAG TGG TGG TGA TGG TGG TGT AGC
AAA GCY CTT TCN AAG CCT TGT C,

and
(xii) gp120 Reverse Const5 for all subtypes, including sequence encoding His-Tag:
   5′-ATA GCG GCC GCC TAG TGG TGG TGA TGG TGG TGT CTT TTT TCT CTY TSC ACC ACT CTY CT-3′ (SEQ ID No. 52), or a derivative thereof lacking the sequence encoding a His-Tag, and/or comprising a sequence encoding a tag,

In a preferred embodiment, the composition comprises 1, 2, 3, 4, 5, 6, 7, 8, 9, 10, 11 or 12 of above nucleic acids.

In a further embodiment, the present invention relates to a composition comprising at least one of following nucleic acids:
(i) Forward gp120 Const1 for A subtype (with XbaI site on 5′ end):
   5′-AAT TCT AGA CRC TRC AGA AAA CTT GTG GGT YAC-3′ (SEQ ID No. 52), or a derivative thereof lacking the XbaI site, and/or comprising a restriction site at the 5′ end
(ii) Forward gp120 Const1 for B subtype (with XbaI site on 5′ end):
   5′-AAT TCT AGA CGC TRC AGA AMA ATT GTG GGT CAC-3′ (SEQ ID No. 69), or a derivative thereof lacking the XbaI site, and/or comprising a restriction site at the 5′ end,
(iii) Forward gp120 Const1 for C subtype (with XbaI site on 5′ end):
   5′-AAT TCT AGA CGT RRT GGG RAA CTT GTG GGT CAC-3′ (SEQ ID No. 70), or a derivative thereof lacking the XbaI site, and/or comprising a restriction site at the 5′ end,
(iv) Forward gp120 Const1 for G subtype (with XbaI site on 5′ end):
   5′-AAT TCT AGA CGC CTC ARA TAA CTT GTG GGT CAC AG-3′(SEQ ID No. 71), or a derivative thereof lacking the XbaI site, and/or comprising a restriction site at the 5′ end,
(v) V1 reverse for all subtypes:

(SEQ ID No. 25)

5′-GCA GTT TTT YAT TTC TYX XXX XXX XXX-3′,

(vi) V2 reverse for subtypes A, D, C:

(SEQ ID No. 28)

5′-AGG TAT TRC AAT TTA TTX XXX XXX X-3′,

(vii) V2 reverse for subtype B:

(SEQ ID No. 29)

5′-CTG AGG TRT TAC AAX XXX XXX X-3′,

(viii) V2 reverse for subtype G:

(SEQ ID No. 30)

5′-AGA CAT TAC AAT TTA TTX XXX XXX X-3′,

(ix) V2 reverse for subtype F1:

(SEQ ID No. 31)

5′-TTG AGG TAT TRC AAX XXX XXX X-3′,

(x) V3 reverse for subtypes A, D, (C):

(SEQ ID No. 40)

5′-AAA GTT TBA TTC CAX XXX XXX XX-3′,

(xi) V3 reverse for subtype B:

(SEQ ID No. 41)

5′-AAA GTG TTR TTC CAX XXX XXX XX-3′,

(xii) V4 reverse for subtype G:

(SEQ ID No. 19)

5′-CAA TTT GTT TTA TYY TAC A XX XXX XXX X-3′,

(xiii) V4 reverse for subtypes A, B, C, D:

(SEQ ID No. 20)

5′-TAA TTT GYT TTA TTY TGC A XX XXX XXX X-3′,

(xiv) V5 reverse for all subtypes:

(SEQ ID No. 15)

5′-TCC TCC TSC AGG TCT GAA XXX XXX XXX X-3′,,

(xv) gp41(160) Reverse for A, B, D subtypes, including sequence encoding His-Tag:
   ATA GCG GCC GCC TAG TGG TGG TGA TGG TGG TGT AG YAA AGC YCT TTC NAA GCC CTG TC (SEQ ID No. 46), or a derivative thereof lacking the sequence encoding a His-Tag, and/or comprising a sequence encoding a tag,
(xvi) gp41(160) Reverse for subtype A, rare variant:

(SEQ ID No. 66)

ATA GCG GCC GCC TAG TGG TGG TGA TGG TGG TGT AGC
AAA GCY CTT TCN GCG CCC TGT C

(xvii) gp41(160) Reverse for C subtype:

(SEQ ID No. 67)

ATA GCG GCC GCC TAG TGG TGG TGA TGG TGG TGT WGC
AAA GCT GCT TCA AAG CCC TGT C,

and
(xviii) gp41(160) Reverse for G subtype:

(SEQ ID No. 68)

ATA GCG GCC GCC TAG TGG TGG TGA TGG TGG TGT AGC
AAA GCY CTT TCN AAG CCT TGT C.

In a preferred embodiment, the composition comprises 1, 2, 3, 4, 5, 6, 7, 8, 9, 10, 11, 12, 13, 14, 15, 16, 17 or 18 of above nucleic acids.

The primers and primer compositions are used for cloning the HIV-1 peptides, which are then part of the HIV-1 vaccine cocktail. Thus, the primers define the 5′ and 3′ ends of the amplified nucleic acids, respectively. It is of course understood, that sequences in the primers encoding His-Tags or sequences introducing an extra restriction endonuclease site are added in addition to the sequences encoding env peptide or protein fragments.

In a further preferred embodiment, the present invention relates to a kit, comprising at least one composition of the present invention and optionally further compounds suitable for PCR. Suitable further compounds are for example a thermostable polymerase and buffers.

The invention further relates to the use of the compositions comprising nucleic acids defined by the above sequences as primers for the production of a HIV vaccine composition.

In a further embodiment, the present invention relates to a method for producing an HIV vaccine composition, comprising the steps of:
a) creation of a library comprising HIV-1 specific antibodies,
b) enrichment for HIV-1-specific antibodies in the library by panning with HIV-1 peptides, in particular native and/or recombinant HIV-1 peptides,
c) multiplying HIV-1 material comprising HIV-1 peptides, polypeptides or proteins,
d) collecting HIV-1 peptides of the multiplied HIV-1 material using HIV-1-specific antibodies of step b) bound to a support,
e) identification and characterization of the HIV-1 peptides obtained in step d) by mass spectrometry, in particular by MS-MS,
f) cloning of fragments of the gp120 gp160 genes encoding the peptides identified in step e),
g) expressing glycosylated env HIV-1 peptides using the results of step in a eukaryotic expression system,
h) purification of the glycosylated env HIV-1 peptides, and
i) production of a vaccine composition,
wherein the glycosylated env HIV-1 peptides in step g) are characterized by:
   (i) the gp120 peptides have a length of 441-541 amino acids and/or a glycosylated protein molecular weight of about 90-115 kDa, and
   (ii) the gp160 peptides have a length of 816-923 aminoacids, and/or a glycosylated protein molecular weight of about 145-175 kDa, and
   (iii) at least 50, preferably at least 70, more preferably at least 100, even more preferably at least 150 different HIV-1 envelop peptides are expressed.

In a preferred embodiment, the vaccine composition of step i) comprises
(a) at least one peptide characterized by the N-terminal sequence L C V T L (SEQ ID No. 72)
(b) at least one peptide characterized by the N-terminal sequence N C S X, wherein X is Y or F (SEQ ID No. 73)
(c) at least one peptide characterized by the N-terminal sequence X I N C, wherein X is K or Q or E (SEQ ID No. 74),
(d) at least one peptide characterized by the N-terminal sequence X I N C, wherein X is T or P or A, (SEQ ID No. 75),
(e) at least one peptide characterized by the N-terminal sequence E F F Y C (SEQ ID No. 76),
(f) at least one peptide characterized by the N-terminal sequence T R D G (SEQ ID No. 77), and
(g) at least one peptide characterized by the N-terminal sequence L D X X E N L W V L D, wherein X is T or A, independently from each other (SEQ ID No. 78).

The invention further relates to a HIV vaccine composition, obtainable by a method of the invention.

The invention further relates to a HIV vaccine composition comprising at least 50, preferably at least 70, more preferably at least 100, even more preferably at least 150 different HIV-1 envelop proteins/peptides, characterized in that:
(i) the gp120 peptides have a length of 441-541 amino acids and/or a glycosylated protein molecular weight of about 90-115 kDa, and
(ii) the gp160 peptides have a length of 816-923 amino acids, and/or a glycosylated protein molecular weight of about 145-175 kDa.

In a preferred embodiment, the vaccine composition further comprises
(a) at least one peptide characterized by the N-terminal sequence L C V T L (SEQ ID No. 72)
(b) at least one peptide characterized by the N-terminal sequence N C S X, wherein X is Y or F (SEQ ID No. 73)
(c) at least one peptide characterized by the N-terminal sequence X I N C, wherein X is K or Q or E (SEQ ID No. 74),
(d) at least one peptide characterized by the N-terminal sequence X I N C, wherein X is T or P or A, (SEQ ID No. 75),
(e) at least one peptide characterized by the N-terminal sequence E F F Y C (SEQ ID No. 76),
(f) at least one peptide characterized by the N-terminal sequence T R D G (SEQ ID No. 77), and
(g) at least one peptide characterized by the N-terminal sequence L D X X E N L W V L D, wherein X is T or A, independently from each other (SEQ ID No. 78).

In a preferred embodiment, the HIV-1 envelop peptides are produced recombinantly.

The peptides may be expressed in an expression system suitable for expressing eukaryotic peptides and proteins, like e.g. in yeast strains, insect cells or eukaryotic cellular parasite systems. In particular, the peptides may be expressed in L. tarentolae, as exemplified in example 4.2 and/or in WO 2009/046984.

In a preferred embodiment, gp120 peptides are produced recombinantly and/or are encoded by nucleic acids having a length of 1323 to 1623 bp.

In a preferred embodiment, gp160 peptides are produced recombinantly and/or are encoded by nucleic acid having a length of 2448 to 2769 bp

In a further preferred embodiment of the invention, the nucleic acids encoding the peptides of the HIV vaccine composition optionally comprise sequences encoding a tag, in particular a His-Tag, and/or sequences encoding a signal peptide suitable for recombinant expression and/or sequences enabling expression in a eukaryotic expression systems.

In particular, the nucleic acids encoding the peptides of the HIV vaccine composition are inserted in a suitable vector for eukaryotic expression. A vector may comprise one or more nucleic acids encoding one or more peptides of the HIV vaccine composition.

The original PCR products for expressing the peptides of the HIV vaccine composition are obtained using primers as described above and tailored in suitable (Leishmania tarentolae) vector in identified between for-rev ends loop, from V1 to V5, respectively.

The immunogenic component of immunogenic composition is expressed in and extracted from suitable eukaryotic host in vivo system, in particular in Leishmania tarentolae (L. tarentolae).

In a preferred embodiment, the HIV vaccine composition is effective as determined by detection of the viral load absence/presence and/or the specificity of an immune response in a body fluid sample of the mouse model of the present invention.

The immunogenic component consists of a number of recombinant HIV-1 envelop peptides/proteins not less than several decades of variants of them with length for gp120 representatives from 1323 to 1623 bp (441-541 aminoacids) and glycosylated protein molecular weight 90-115 kDa, and with length for gp160 representatives from 2448 to 2769 bp (816-923 aminoacids, respectively) and glycosylated protein molecular weight 145-175 kDa.

A “tag” according to the present invention is a peptide sequence which is suitable for isolation, and which is covalently attached to a peptide or protein sequence of interest. In a preferred embodiment, the tag is a His-Tag. A His-tag is a peptide sequence consisting of six Histidine-residues. The His-Tag binds to metal matrices. Other suitable tags are chitin binding protein (CBP), maltose binding protein (MBP), and glutathione-S-transferase (GST) and FLAG-tag.

According to the examples, a His-Tag was used, for purifying the expressed peptides. However, also other tags, in particular affinity tags may be used for purifying the peptides.

Also, several primers of the invention comprise a XbaI site. This site suitable for cloning amplified PCR products into suitable expression vectors. It is understood that also other restriction sites are suitable for cloning PCR products into expression vectors.

The creation of a humane recombinant IgG phagemid library containing HIV-1 specific scFv antibody fragments and the enrichment for HIV-specific scFv antibody fragments in the phagemid library by panning with native or recombinant HIV-1 peptides is described in examples 2.1, 2.2 and in WO2009/046984.

Methods for multiplying HIV-1 material comprising HIV-1 peptides, polypeptides or proteins is described in WO 2009/046984 and in the present invention. In particular, HIV-1 may be amplified in PBMC culture cells.

Collecting HIV-1 peptides by reverse panning of the multiplied HIV-1 material using the enriched HIV-1 phagemid library described above bound to a support is described in example 2.3 and in WO 2009/046984. The identification and characterization of the HIV-peptides thus obtained is described e.g. in WO 2009/046984 and in examples 3.1 and 3.2 of the present invention.

The identification and characterization of the HIV-1 peptides by mass spectrometry, in particular by MS-MS, more preferred by LC-MS-MS is for example described in example 3.

Expressing glycosylated env HIV-1 peptides using the results of above step in an expression system, is described in WO 2009/046984 or in examples 4.1, 4.2 and 4.3 of the present invention.

The purification of the glycosylated env HIV-1 peptides is described in WO 2009/046984 and in Example 4.3 of the present invention.

The production of a vaccine composition is described in WO2009/046984.

In a preferred embodiment of the method of the present invention, the individuals, from which the HIV material is obtained are infected by the same or a different HIV subtype.

In a preferred embodiment of the method of the present invention, the individuals, from which the viral material is obtained are antiretroviral therapy naïve patients or patients that have been subjected to antiretroviral therapy.

In a preferred embodiment of the method of the present invention, the phagemid library is prepared by the steps of
a) preparing DNA-fragments derived from nucleic acids encoding the variable region of a light chain and a heavy chain, respectively, of immunoglobulines expressed in B-Lymphocytes obtained from a number of individuals infected with HIV,
b) linking the DNA-fragments encoding the immunoglobuline light and heavy chain, to allow expression of a polypeptide, comprising the variable regions of a light chain and heavy chain, respectively, of immunoglobulines, to create a multitude of different specificities,
c) cloning of linked fragments in phagemid vector and transforming bacterial strain for expression on the bacteriophage's surface, preferably wherein amplification is carried out with any of the primer combination listed in tables 1-7 of WO2009/046984, more preferably wherein obtained scFv phagemid recombinant antibodies are specific to resistant HIV variants carried out in HAART- or any other antiretroviral therapy experienced patients.

In a preferred embodiment of the method of the present invention, step a) further comprises an enrichment of the phagemid library presenting antibody's scFv fragments in panning procedure binding HIV-specific antibodies with recombinant gp120-, gp41- and native HIV-polypeptides isolated from different donors.

In a preferred embodiment of the method of the present invention, LC mass spectrometry is applied for a quantitative analysis, identification and sequencing of HIV-1 gp120 and HIV-1 gp41 and its standard and variable fragments.

In a preferred embodiment preparing a HIV preventive vaccine composition is accomplished by addition and/or conjugation of optional immunogenic stimulants, adjuvants or carriers, such as sterically stabilized liposomes (SSL).

In a preferred embodiment, the present invention relates to a HIV vaccine composition of the present invention further comprising immunogenic stimulants, adjuvants or carriers, such as sterically stabilized liposomes (SSL).

The present invention also relates to the use of a HIV vaccine composition of the present invention for immunization of non-infected individuals against catching and development of HIV infection and AIDS disease.

### FIGURE LEGENDS

FIG. 1 shows samples of preparative PAAG electrophoresis with or without marked bands for cutting for trypsinolysis with further mass spectrometry analysis:
A—PokA-79-PBMC and U455-PBMC samples (FIL-1 and FIL-2, respectively), Coumassi staining;
B—H9/IIIB and PokA-79 laboratory strains samples, Silver staining.

FIG. 2 shows SCID mice endogenous immune deficiency testing compared to BalbC response for immunization with isolated U455 laboratory strain proteins.

FIG. 3 shows SCID-PBMC and BalbC immune response for immunization with cocktails of recombinant p120-1 and p120-2 proteins.

FIG. 4 shows PokA-79 viral load rates on U937 and MT-4 background, 3 challenges 7 days b/w period.

FIG. 5 shows PokA-79 viral load rates on U937 and MT-4 background, 2 challenges 3 days b/w period.

FIG. 6 shows PokA-79-MT4 and U937-background's viral load rates for different periods between challenging in SCID mice.

FIG. 7 shows PokA-79 strain in vitro replication dynamics, lymphocyte MT-4, monocyte U937 cultures, PBMC and DC background.

FIG. 8 shows PokA-79 strain in vitro replication dynamics, p24 expression, lymphocyte MT-4 and monocyte U937 background.

FIG. 9 shows the morphology of cell culture and HIV-1 strain infection manifestation on the 3^{rd} day of in vitro cultivation. Syncithyum formations are marked with arrows.

A) MT-4 lymphocytes

### B) PokA-79-MT4

FIG. 10 shows the morphology of U937 monocytes culture and HIV-1 strain infection manifestation on the 5^{th} day of in vitro cultivation. Syncithyum formations are marked with arrows.

A) U937 intact monocytes, B) PokA-79-U937 and C) U455-U937

FIG. 11 shows the morphology of PBMC and HIV-1 strain infection manifestation on the 5^{th} day of PBMC passage in vitro cultivation. Syncithyum formations are marked with arrows.

A) Healthy donor PBMC
B) PokA-79-PBMC 6^{th} passage (6 weeks)
C) U455-PBMC 5^{th} passage (5 weeks)

FIG. 12 shows the morphology of DC and PokA-79-DC strain cultivation on the 3^{rd} day of DC passage—the is no visible difference between donor dendrite cells and chronically infected with PokA-79 DC at 5^{th} passage.

A) Healthy donor DC
B) PokA-79-DC 5^{th} passage (5 weeks)

FIG. 13 shows Laboratory strains U455 and H9/IIIB viral load rates on MT-4 and U937 cell cultures background, 4 challenges with 7 days b/w period.

FIG. 14 shows U455 and H9/IIIB in vitro replication dynamics on lymphocytes MT-4 and monocyte U937 backgrounds.

FIG. 15 shows U455 and H9/IIIB in vitro replication dynamics, p24 expression, lymphocyte MT-4 and monocyte U937 cultures.

FIG. 16 shows PokA-79 viral load rates on cell culture U937 and MT4 background depending on the number of challenging.

FIG. 17 shows SCID-PBMC and SCID-cell culture background viral Load rates for challenging with PokA-PBMC and PokA-79 strains, respectively.

FIG. 18 shows negative challenging results for dendrite cells (DC) and Fresh PBMC PokA-79-U937 and U455-U937 lab. strains (PBMC 1st week) compared to 6-8th PBMC passage and U937 background.

FIG. 19 shows PokA-79 in vitro passages replication dynamics, DC and PBMC background, 1st week and 5-6th weeks passages.

FIG. 20 shows U455 and PokA-79 in vitro replication dynamics, dendrite cells (DC) or PBMC background, 5th and 6th passages.

FIG. 21 shows:
A) Scaffold software HIV Envelop protein's identification and trustworthy analysis,
B) HIV Envelop protein's spectra,
C) HIV Envelop protein's triptic peptides mapping,
D) Scaffold software HIV Envelop protein's identification and trustworthy analysis,
E) HIV Envelop protein's triptic peptides mapping.

FIG. 22 shows the HIV Envelop protein's triptic peptides mapping.

FIGS. 23 A) to 23 M) show t:he HIV Envelop protein's spectra.

FIG. 24 shows primers disposition for PCR products of immunogenic composition's tailoring mapped for several sequences in alignment.

FIG. 25A) shows immune responses for gp160-1 and gp160-2 in challenged with PokA-79-PBMC strain SCID-PBMC Mice.

FIG. 25B) shows recombinant gp160-1 and gp160-2 immunizations effect for SCID-PBMC challenging with PokA-79-PBMC 6th passage strain.

FIG. 25C shows recombinant gp160-PokA-PBMC and gp160-U455-PBMC immunization effect for challenging with PokA79-PBMC and U455-PBMC 6-8 weeks strains.

FIG. 26 shows A) the scheme of constant and variable regions in HIV-1 gp120 and primers arrangement, and B) Gp160 schematic map and primers arrangement.

### THE STATE OF THE ART

The present invention relates to the animal model for HIV vaccine effectiveness evaluation and novel HIV vaccine compositions. In particular the presented method comprises endogenous immune system deficient CSID-humanized mouse model application for testing preventive HIV vaccine's immunization effect for in vivo HIV challenging prevention. SCID humanized peripheral blood lymphocytes (SCID-PBMC) or SCID humanized dendrite cells-engrafted mice (SCID-DC-PBMC) were used for immunizations and following challenging with HIV laboratory strains which envelop protein's pool was obtained according to reverse panning and mass spectrometry analysis method. One donor PBMC or unmixed one donor DC-PBMC derivations were inoculated into one animal.

Severe Combined T-B-Immune Deficient (SCID) and thymus-free nude mice were never used in practice for in vivo evaluation of HIV vaccines effectiveness in spite it was known since early 90-ies that such mice can be successfully engrafted with human peripheral blood (PBMC), dendrite (DC) and stem cells believed to be capable for anti-HIV immune response formation [1, 13, 16] and in vivo HIV infection development [2, 3, 9, 12, 22]. An idea to combine these two features and to use hu-SCID mice for vaccination assays was discussed [9]. Preclinical assays of DNA- and peptide-based HIV vaccine forms were run in a very limited number of animal models, mostly in two models only, i.e. in Rhesus Macaques and Chimpanzees (Chimps). In Rhesus Macaques human HIV infection does not work, so vaccines were tested exclusively for eliciting HIV-specific antibody responses detecting with extremely sensitive ELISPOT assays. In Chimps that are bearing HIV infection but without developing any symptoms of acquired immune deficiency syndrome (AIDS) finally test results were resumed to the same ELISPOT data. Therefore the existing monkey models are not at all satisfying since HIV infection does not at all develop in these animals like in humans. In general vaccines are distinguished from other therapeutic/preventive medical agents with their luck of the first stage of research in vitro in positive effect testing.

There are two main vaccine generations from beginning research in a vaccine development to finish with large scale medical purpose production. The first generation is represented by natural pathogen or its derivations deactivated with chemical compounds treatment and further neutralization of possible toxicity, fermentative pathogen's lysis into peptides and proteins, sterilization via autoclaving or any other similar method for vaccine active component's conservation. This way of vaccines creation was running for XXth century successfully for all bacterial pathogens like tuberculosis, plague, diphtheria, and also for large, stable DNA viruses diseases as smallpox, stable single- and double-strand RNA viruses diseases like morbilli, hepatitis A and B, human encephalitis, rabbies and other more rare tropical disease's vaccines. The first class of vaccines normally provides the formation of pathogen-specific, elongated and strong immune responses sufficient for infection development prevention. However the backwards of this vaccine's generation always were the lack of safety in administration due to high risk of survived pathogen's activity causing the development of real disease instead of immunization against it, and also anaphylactic reactions of immune system to foreign proteins and further immune vulnerability and disorders.

The second generation of vaccines development started with contributions of genetic engineering and molecular biology as recombinant proteins/peptides-based vaccines that are replacing the first class since the late 80-ies. Recombinant vaccines were quickly accepted since they provide a higher degree of safety in administration and production, since they are free of disease-evoking pathogens [14, 20]. In case they were targeted to conservative and stable pathogens after several years of efforts in development they matched highly specific immunity and close to complete protection against infectious disease. Recombinant proteins or peptide's mixtures produced in cheap and easy-to-maintain prokaryotic expression systems (E. coli transformed strains) are able to make exactly the mimic representation of prokaryote pathogen surface “checkpoints” crucial for antibodies recognition and vaccination against a number of bacterial infectious diseases. Bacterial cell surface normally contains a standard number of very conservative proteins, totally about one thousand. The similar situation with standard surface antigens exposition and their number is observed for many large DNA and RNA pathogenic viruses of different families like coronaviridae, adenoviridae, poxyiridae, etc. Some small RNA viruses also have very stable genotype spread in its host practically all over the world—for example picornaviridae hepatitis A virus. For each known recombinant vaccine in history years were spent to solve the difficulty of distinguishing these protein's “checkpoints” responsible for immunity against the current pathogen from several hundred of possible candidates. Then immunogenic peptides cocktail preparation and pre-clinic efficacy assays took some more years of efforts. Within next several years of clinical trials doctors studied and demonstrated the advantages of recombinant vaccine over native pathogen's derivatives administration. This process normally took 15-50 years for every individual recombinant vaccine development.

If we try to transfer an HIV vaccine development attempts to this scheme of two stages described above it becomes obvious immediately that HIV vaccines do not fit there. There are several main reasons for it. First of all, the development of anti-HIV vaccine based on native pathogen described in several patents from early 90-ies [5, 11] was not possible due to impossibility to produce and collect the viral substrate in somehow reasonable amounts. In our research we certainly showed that it was not possible to concentrate virus envelop proteins with standard sucrose gradient untracentrifugation methods with over 200000 g gravitation speed even in amounts sufficient for analytical purpose [7].

Therefore the first stage was absent for HIV vaccine, the second stage—recombinant vaccines development attempts—was tried [8, 14]. The virus genome sequencing prior to attempts to create recombinant versions revealed so great variability of about one third viral genome that it was poor chances to find two identical full-length variants even from the same sample of one patient or group of patients mixed together [15].

As a result now modern spectrum of HIV vaccines issued for clinical trials is represented with DNA vectors form—in terms of vaccines development evolution it is an incomplete recombinant second stage which did not yet reach a protein composition necessary for vaccination effect. So DNA HIV vaccine preclinical assays were run in Rhesus Macaques and Chimpanzees.

Meanwhile the animal model for studying human HIV infection and its treatment with antiviral agents was originally created in the late 80-ies by Derek Mosier [13] and improved by several laboratories in the world for different practical applications in HIV research [2, 6, 9, 10]. SCID mice engrafted with human leukocytes (PBL) provided human-like immune responses for immunization with different antigens including HIV peptides and proteins [1, 2, 4, 6]. Animals were supplied with 20-50×10⁶ PBL intraperitoneally in the age of 2-5 weeks [9, 10], for the next two months they were examined for presence of human lymphocytes in peritoneal cavity, spleen, lymph nodes, liver and for human-like cytokine's profiles in bloodstream. After two-three months lymphocytes depletion, anemia, in some cases—leukemia development, were observed in grown up animals [9, 16]. To avoid such side effects (graft versus host disease—GVHD) it was offered to engraft immune deficient mice with dendrite cells produced via treatment of human leukocytes with human IL-2, IL-12, INF-γ before engrafting [6, 9, 10]. Some researchers engrafted newborn CSID mice with parts of human organs and tissues—spleen, thymus, dendrite cells [19]. One human donor organ's tissue engrafting for a single mouse was successfully made by Stoddart group [18].

Different methods were adapted for modeling HIV infections in SCID and NOD-SCID mice, 95 percent of these experiments were based on laboratory HIV strains cultivated in vitro at cell cultures. CD4, CCR5 and CXCR4 receptors expressing cultures such as transfected HeLa-CD4, MT2, MT4, MAGI-CCR5, U937 and so on were used as an engrafted substrate for viral infection [10, 21]. Grafts of human tissues (Hu-SCID Thy/Liv), isolated human leukocytes (PBL) and their infected with laboratory strain variants were administered intraperitoneally and provided HIV infection [2, 11, 22]. Infection rates were measured with RT-PCR or Real-Time PCR from intraperitoneal lavage, liver, spleen and thymus area [9]. But more often SCID mice are engrafted with dendrite cells cultivated in vitro with interleukin factors from human peripheral blood leukocytes (PBL). Such humanized chimerical mice are reported to be able to bear HIV infection for long periods of time (1-6 months) [3, 9] and were used for testing and demonstration of antiviral activity for different classes of antiretroviral agents [9, 17, 21]. It was demonstrated that engrafted with dendrite cells animals normally are protected from development of graft vs host disease and live for months after the xenograft embedding [22].

A number of researchers especially from those who was convinced in clinical trials HIV vaccines they developed themselves do not work proclaimed it is not possible to invent HIV immunization vaccine which will protect humankind from viral infection catching and development [17]. Some experiments with dendrite cells cultivated in vitro in presence of HIV laboratory strain cell culture or cultivating media and then engrafted into SCID or NOD-SCID mice showed these animals do not catch and do not maintain HIV infections from challenging with the same laboratory strain [9]. The same experiment with dendrite cells engraftment into SCID mice and blockage of infection with natural HIV isolates cultivated on healthy donors PBLs was also described once [9].

Within the present invention, however, it is understood that the other immune deficient laboratory animals such as nude mice, immune deficient rats, immune deficient cats, etc. can be used.

Severe combined T-B-immune deficient mice (SCID mice) and methods for their production are known in the art. Furthermore, SCID mice are also commercially available. However the production of hu-SCID mice with presence of blood serum-detectable immune response to HIV envelop proteins and HIV challenging blood serum-measured viral load as a number HIV RNA copies per ml of serum of non-immunized mice with the same PBMC background was never described. In spite of high HIV-specific immunogeniety and declared HIV challenging ability nobody ever published results that SCID mice were used for testing DNA HIV vaccines efficacy [2, 6, 9, 10]. Only one of all developed hu-SCID mice models—grown up SCID off-springs with engrafted in life first 1-3 weeks human thymus and liver (hu-SCID Thy/Liv)—proved to be able to bear both HIV-specific immune response and the viral challenging in the bloodstream of the same animals [2, 11, 22]. However such humanized mice production is greatly labor-consuming and highly expensive. The cost of one human thymus and liver-humanized SCID mouse exceeds the cost of one small primate, such as Macaque Rhesus monkey, being used for laboratory experiments.

It is advantageous that Hu-SCID mice can provide a quick generations replacement due to the short lifespan, and also a valid statistical analysis because little 15-25 gram body weight animals need approximately 50 times less immunization material for testing each than Macaques with several kilos body weight. A real possibility to provide mice with enough immunocompetent human cells material for engraftment is also worth for consideration.

### DETAILED DESCRIPTION OF THE INVENTION

The present invention demonstrates that Hu-SCID mice represent an efficient animal model for the evaluation of an HIV vaccine.

Within the present invention, however, it is understood that also nude mice, immune deficient rats, immune deficient cats and other immunodeficient animals can be used.

Severe combined T-B-immune deficient mice (SCID mice) and methods for their production are known in the art. Furthermore, SCID mice are also commercially available.

Techniques for engrafting SCID mice with human immunocompetent cells are also known in the art and are, additionally, described in the attached example.

As used in the present invention the term “immunocompetent cells” comprises any cell of the specific immune system, such as lymphocytes and in particular T and B lymphocytes, which have been exposed to a particular antigen of interest, i.e. the HIV-vaccine to be evaluated, in vitro or in vivo. Examples for T-lymphocytes (T cells, thymus cells) are T-helper cells, cytotoxic T cells, NKT-cells, γδ-T cells, and memory T cells. T cells may be CD4 and/or CD8 positive and/or negative respectively, depending on their maturation status. Examples for B lymphocytes (B cells, bone cells), include plasma B-cells, B1- and B2-cells, marginal zone B cells and memory B cells. Immunocompetent cells may be obtained e.g. from individuals, such as peripheral blood mononuclear cells (PBMC), or from cell lines.

Preferably, the immunocompetent cells may be selected from the group consisting of PBMC, dendritic cells, B-cells, and T-cells. More preferably, the human immunocompetent cells are PBMC or dendritic cells.

Introduction of cells of human origin into the SCID mice may be achieved by using any conventional route, such as via the intraperitoneal or parenteral route. The intraperitoneal route is normally preferred due to the ease of administration and allowing administration of a volume of fluid. The cells may be introduced in form of a suspension in any suitable medium, allowing cell survival, such as phosphate buffered saline PBS or Dulbecco's modified Eagle's medium (DMEM), Roswell Park Memorial Institute medium (RPMI) or Glasgow's Minimal Essential Medium (GMEM).

The immunocompetent cells may be stimulated with immune stimulators prior to being introduced into the SCID mice. Immune stimulators for such purpose may be the immune stimulators mentioned above. Preferred stimulators include but are not limited to interleukin factors such as IL-2 (interleukin-2), IL-4 (interleukin-4), IL-12 (interleukin-12), and other factors such as INF-γ and GCSF (granulocyte colony stimulating factor). Further or different factors or combinations of such factors may also be used. The immune stimulators promote growth and/or development of e.g. T, B, and hematopoietic cells.

The dendritic cells may have been obtained by culturing human PBMC in the presence of cytokines capable of inducing the formation of dendritic cells in vitro. Methods for the production of dendritic cells in vivo are known in the art (see e.g. [25]).

The cell count for each introduction of cells of human origin may vary from about 1×10³ to 1×10⁸ cells per introduction, preferably from about 1×10³ to 1×10⁷ cells per introduction, more preferably from about 1×10⁴ to 1×10⁷ cells per introduction, even more preferred from about 1×10⁵ to 1×10⁷ cells per introduction, and even more preferred about 1−15×10⁶ cells per introduction (i.e. for a single engraftment only). The introduction of cells of human origin may be repeated prior to step (b), such as two to three times once weekly, or twice weekly, or providing a pause prior to a third or further introductions.

In case PBMCs are introduced into SCID mice followed by immunization these mice are referred to as “PBMC-Hu-SCID mice”. In case dendritic cells (DCs) are introduced into SCID mice followed by immunization and subsequent introduction of cells of the immunogenic background such as PBMCs, these mice are referred to as “DC-PBMC-Hu-SCID mice”.

Preferably, the immunocompetent cells intended for one mouse are from one human donor. In case that various mice are used, it is preferred that for one assay, the immunocompetent cells are derived from one human donor and are then engrafted into the different mice.

As discussed above, the present invention relates to a method for the evaluation of an HIV vaccine, wherein a Hu-SCID-mouse as defined above is inoculated with the HIV vaccine and thereafter challenged with HI-virus.

The vaccine may be applied/introduced in form of a suspension or mixture in any suitable medium and the exposure (vaccination) may be carried out in the presence of one or more adjuvants such as one or more pharmacological or immunological agents that modify the effect of the HIV-vaccine, such as immunopotentiators—also known as boosters of the immune system—which are effective in stimulating cell-mediated immunity. A preferred adjuvant for this purpose is Freund's adjuvant.

For an in vivo vaccination the vaccine to be tested may be administered directly to the mice on the day following the last introduction of immunocompetent cells or of the cells of the immunogenic background, or it may be administered some time (e.g. a couple of days or even weeks, preferably after two weeks) after the last introduction of said cells. Immunization may be carried out once or in a number of subsequent steps, e.g. it may be repeated after some time, e.g. after one to three weeks, preferably after two weeks two to four times, preferably three times.

The Hu-SCID-mouse may be inoculated with the HIV vaccine 1 to 3 weeks after engrafting of the human immunocompetent cells.

Administering the vaccine to the mice may be performed using any route considered suitable, e.g. via the parenteral route or via the intraperitoneal route such as intravenous injection, subcutaneous injection, or intramuscular injection. The preferred route for administering the vaccine to the mice is via subcutaneous injection.

The vaccine to be tested may be administered to the Hu-SCID mice once, twice or more often (three to six times). Preferably, the Hu-SCID mice are vaccinated for a total of four times within two to four weeks, preferably within three weeks.

In case that the vaccine is a peptide or protein, the amount of vaccine peptides or proteins administered to the Hu-SCID mice may be between 1 and 100 μg, preferably between 10 and 80 μg, more preferably between 15 and 50 μg, even more preferably between 20 and 40 μg and most preferably about 25 μg of vaccine peptides or proteins in each step for each mouse. Administering may be performed in one or more individual steps, interrupted by lag phases lasting one day, days or weeks.

The infection of the hu-SCID mouse be performed by using an amount of HIV strain infectious doses in cell culture, e.g. from about 5×10² to 5×10⁶ TCID₅₀ (50% Tissue Culture Infective Dose), preferably from about 5×10² to 5×10⁴ TCID₅₀. For example, for infecting Hu-SCID mice having freshly isolated PBMCs or DCs as background, from about 5×10³-10⁴ TCID₅₀ of HIV-1 laboratory strain infectious doses in cell culture are administered to each individual mouse, or from about 5×10²-5×10³ TCID₅₀ infectious doses. Suitable cell cultures for this purpose include but are not limited to MT-4 or U937 culture cells. Preferably Hu-SCID mice can be challenged with 5×10³-10⁴ TCID₅₀ of HIV-1 laboratory strain infectious doses in MT-4 culture cells, or with from about 5×10²-5×10³ TCID₅₀ infectious doses in U937 culture cells.

It will be understood that different ranges of cell numbers and TCID₅₀ may be used for infecting/exposing the Hu-SCID mice with/to HIV. Also, the HIV strains used for this purpose are not limited to a specific strain. Rather, all known HIV strains including HIV-1 strains and HIV-2 strains may be used as long as it is made sure that unprotected immunocompetent cells/mice (i.e. cells/mice not vaccinated with the HIV-vaccine to be tested) will be successfully infected with HIV. Preferably the HIV strain is an HIV-1 strain. More preferably the HIV strain is selected from the group consisting of U455, 9H/IIIB, and PokA-79.

In particular, an infectious dose of 5×10²-10⁴ TCID₅₀ or higher for challenging of one Hu-SCID animal may be used. Preferably, infectious doses of at least 10⁴ TCID₅₀, more preferably of at least 10⁵ TCID₅₀, even more preferred of at least 10⁶ TCID₅₀ are used. Preferably, infectious doses of up to 10⁷ TCID₅₀ are used.

“HIV laboratory strain playback” according to the present invention is understood as an HIV laboratory strain obtained by culturing a HIV laboratory strain in PBMC cells of a healthy donor for several weeks passages, in particular for at least about 2 weeks passages, and up to about 10 weeks passages.

In a preferred embodiment, at least one HIV laboratory strain playback is used to infect and/or expose the Hu-SCID mice with/to HIV.

As an alternative to challenging Hu-SCID mice with an amount of HIV strain infectious doses in cell culture, simultaneous challenging of Hu-SCID mice with a mixture of infected and uninfected cells may be performed. Such a mixture of infected and uninfected cells may comprise from about 1×10⁴ to 10×10⁸, preferably from about 1×10⁵ to 1×10⁸ and most preferably about 10×10⁶ of pure (uninfected) cells and from about 1×10⁴ to 10×10⁷, preferably from about 1×10⁶ to 1×10⁷ and most preferably about 5×10⁶ cells infected with an HIV strain. In a preferred embodiment SCID mice are challenged with e.g. 5-10×10⁶ MT-4 or U937 pure culture cells and with 5×10²-10⁶ TCID₅₀ of HIV-1 laboratory strain infectious doses in cell culture. Preferably the mice are challenged with 5×10³-10⁴ TCID₅₀ of HIV-1 laboratory strain infectious doses in MT-4 culture, or with 5×10²-5×10³ TCID₅₀ infectious doses in U937 culture.

The challenges with HIV may be repeated for a number of times, e.g. for 2-5 times, preferably for 2 to 3 times with different time periods in between. A 3 day period between challenging is preferred for the achievement of the high viral load numbers in a lymphocyte background culture and a 7 day period is preferred for a high viral load in a macrophage background culture.

For measuring the efficacy of a vaccine, all methods generally known in the art for that purpose may be used. In particular, the efficacy of the HIV vaccine may be determined by determining the viral load in a body fluid sample of the mouse. Said body fluid sample may be blood serum.

For determining the viral load every method known in the art can be used. This includes Real Time PCR, RT-PCR and ELISA.

The figures, scientific work and examples provided herein are intended to illustrate, but not to limit the invention.

1. HIV Envelop Proteins Variability Modeling Analysis.

In the very beginning of the current immunogenic composition development we tried to estimate a number of possible variations in main envelop protein gp120 for cohorts of patients applying new mathematics modeling based on differential analysis and molecular biochemistry parameters [23, 24]. The length of gp120 nucleotide sequence is variable and depends on deletions and insertions presence, their number and size. There is a scheme of gp120 mapping, where C1-C5—conservative regions; V1-V5—variable loops or variable regions.

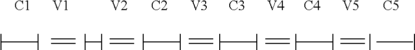

According to formula:

$$ln N = {\sum }_{i , j}^{ } {α}_{i , j} ln {C}_{{n}_{i}}^{j} ,$$

where i—is number of variable nucleotide positions in env gp120 sequence, nᵢ—number of matching sites in each of i variable regions (in our case i=5, as variable regions of gp120 are V₁, V₂, V₃, V₄, V₅), j—number of particles, that can be placed. In our case there are four particles—AT, GC and TA, CG.

According to experimental data total number of possible positions, in which nucleotides (A, T, G, C) can be allocated, is N_{box}=NᵥₐᵣN_{del}. Number of components—nucleotides, that will be allocated in boxes is Nₚ=4.

We can estimate variations number by calculating number of combinations by which can be realized states of these Nₚ=4 components in total number of boxes N_{box}:

$${C}_{{N}_{box}}^{Np} = \frac{( {N}_{box} ) !}{( {N}_{box} - {N}_{p} ) ! ( {N}_{p} ) !} \qquad [ 23 ]$$

Length of gp120 sequences used in our alignments is 1443 bp.

Alignment 1—HIV-1 subtype A infected 30 individuals from Former Soviet Union territories and Africa. Nucleotide substitution positions Nᵥₐᵣ=546, there are deletions in 10 positions. Constant positions 887. Total number of positions=1443. For calculation of possible variants number we must summarize number of nucleotide substitution positions and number of deletions. Altogether: N_{box}=546+10=556.

$${C}_{556}^{4} = \frac{556 !}{552 ! \cdot 4 !} = 3939049415 \approx 3 , 9 \cdot {10}^{9}$$

Alignment 2—HIV-1 subtype A infected 10 individuals from Former Soviet Union territories. Nucleotide substitution positions Nᵥₐᵣ=331, there are deletions in 30 positions. Constant positions 1082. Total number of positions=1443. For calculation of possible variants number we must summarize number of nucleotide substitution positions and number of deletions. Altogether: N_{box}=331+30=361.

$${C}_{361}^{4} = \frac{361 !}{357 ! \cdot 4 !} = 695946630 \approx 6 , 9 \cdot {10}^{8}$$

Researchers tried to overcome such genetic diversity for many years fishing out, cloning and reproducing fragments of the most conservative proteins or their domains. However it occurred that internal viral peptides like nef, pol and gag have no connection with virion's infectious activity and immunogenicity against it, and envelop proteins conservative fragments are small enough and are always covered with “loops” parts from outside of invading new host cells virions. These “loops” envelop peptides fragments were not only highly variable but extremely quickly mutation-altering regions at least up to now, until sequence information was obtained from standard infected individual's lymphocyte's DNA sequencing.

It is known from published and our practical experimental data that HIV infectious dose (TCID) and HIV viral load (virus titre or a number of RNA copies per 1 ml of body fluid sample) are very much different numbers. Precisely, TCID value is the thousands times lower number than the viral load. We believe this difference represents the real percentage of infectiously active virions that matches to a number of CD4, CCR5 and CXCR4 receptors variations available for virus recognition and cell entrance in a single human organism subjected to HIV infection danger. An eukaryotic genome is very conservative and stable characteristics. The whole HIV great genetic variability calculated above cannot be repeated in any human genome for CD4 and two more receptors including the number of these proteins isoforms. Therefore the number of HIV infectiously active variants with certain env sequences is limited by the number of tissue-specific variations of CD4 receptor and co-receptors. The identification of these viral env variants is the answer for question about preventive HIV vaccine creation.

2. Viral Envelop Proteins Material Collection with Reverse Panning and Native Human Antibodies Libraries Affine Separation Techniques

We suppose mass spectrometry is the best possible method which can provide quantitative and sequence analysis of HIV envelop proteins for the antiviral vaccine development. We suppose the method was never applied for gp120 and gp160 study due to inability to collect any sufficient biomaterial amount for envelop proteins identification. We have used two stages of envelop material collection from laboratory strains culture media, patients isolates and from laboratory strains put back into “isolates” conditions for cultivation during at least 5 weeks:

1. Virus ultracentrifugation concentration;
2. Reverse panning and native human antibodies libraries affine separation.

2.1. Virus Ultracentrifugation Concentration

Three methods of virus ultracentrifugation concentration were used. According to the first method, HIV proteins were harvested from supernatants by PEG precipitation, resuspended in TNE buffer (0.01 M Tris-HCl pH 7.2, 0.1M NaCl, and 1 mM EDTA in Milli-Q water), and clarified by centrifugation for 30 min at 4,000 rpm. About half of total viral sample volume in TNE buffer was stratified over 20% sucrose solution pored to the bottom of ultracentrifuge tubes and ultraspinned at 160000 g during 45 minutes through sucrose gradient with density 1.16-1.18 g/sm³. The virus-containing pellets were pooled, diluted approximately 1:3 with TNE buffer.

According to the second method, supernatant was run spinning at 1000 g for 15 minutes, then the obtained supernatant is run spinning at 16000 g for next 15 minutes, then stratified over 20% sucrose solution pored to the bottom of ultracentrifuge tubes and ultraspinned at 160000 g during 45 minutes through sucrose gradient with density 1.16-1.18 g/sm³. The pellet is dissolved in small volume of culture media.

The third ultrafiltration method is reasonable for not loosing surface envelop glycoproteins in sucrose gradient and keep them without any denaturating effect. Supernatants were run through ultrafiltration with 30 kDa membrane (Pelicon device Biomax) at 4 C for 2 hours. Filtered hydrolyzed protein mixture was lyophilized. Viral RNA concentration in collected pellets was measured by Real time PCR using Amplisense HIV-monitor-FRT (Amplisense) on Rotor-Gene 6000 thermal cycler (Corbett Research). Protein concentration in the virus-containing fractions was determined by BCA assay. Protein material pellets were stored at −80° C. after dilution and measurements.

The first and the third approaches—PEG precipitation and ultrafiltration—previous to ultracentrifugation and PAAG protein bands separation showed reasonable results in further mass spectrometry analysis.

2.2. Reverse Panning Libraries Affine Separation

The combination of reverse panning technique described in [7] and native antibodies libraries affine chromatography was used for HIV envelop proteins further concentration.

For phagemid library presenting recombinant phage production M13KO₇ helper phage is added to overnight TG1 E. coli culture for 1 hour pre-incubation and 12 hours incubation in presence of 100 μg/ml ampicillin and 50 μg/ml kanamycin at 37° C. (the typical phage yield is 10¹⁰ to 10¹¹ ampicillin-transducing units per ml). The culture is spinned at 1000 g for 10 min., supernatant was collected and cooled. Then ⅕ v/v of PEG8000/NaCl (20% PEG/2.5M NaCl) solution is added to supernatant and incubated 1 hour at ice, then precipitation performed with spinning 10000 g at 4° C. for 20 minutes. The pellet is dissolved in LB or 10 MM TrisHCl pH 8.0 and filtered through 0.45 μm. Recombinant phage can be stored at 4° C. overnight when 0.01% timerosal is added.

Phage presented mAb libraries were immobilized on streptavidine-coated ferroxide magnetic nanoparticles. Particles were washed with sterile water and equilibrated with 0.1M carbonate buffer, pH 9.5 at a flow rate of 1 ml/min. M13 phages in concentration 2.5*10¹⁰ in 10 ml 0.1M carbonate buffer pH 9.5 were immobilized on streptavidine by re-circulating phage coupling solution for 16 h at a flow rate of 1 ml/min at 4° C. using peristaltic pump. After coupling the phages particles were washed with 0.1M carbonate buffer pH 9.5 until all non-bound phages were washed away. The remaining active streptavidine groups were blocked by re-circulation of 25 ml 0.1 M ethanolamine in 0.1 M carbonate buffer pH 9.5 for 3 h at a flow rate of 1 ml/min. The suspension was washed with sterile water and phosphate-buffered saline (PBS) and stored at 4° C. until further use. 8. To estimate immobilization yield, 100 μl of suspension was sonicated and total protein concentration was determined by micro BCA assay. To converse protein concentration into phage concentration, calibration with control phage particles was performed.

HIV-1 peptides mixture was proceeded through reverse panning with current technique:

HIV-1 peptides mixture hydrolyzed in 0.05 M Tris-HCl, pH 8.0 buffer was pored into 50 ml glass tubes together with phage-embedded streptavidine particles and stirred for 2 hours in orbital shaker with speed 120 r/min. Then the suspension was diluted with 5 volumes of the same buffer. AC magnetic field conductor was applied to collect the particles. After supernatant was discarded HIV peptides-bound phage was eluted with 0.1M glycine pH 2.2 gradient. Obtained fractions were incubated in glycine elution buffer with presence 0.001 M PMSF for 5 hours at RT until phage-antigen complexes are re-adjusted completely.

2.3. Native Human Antibodies Libraries Column Preparation and Pool of HIV Envelop Proteins Capturing.

Native human antibodies libraries were extracted from a cohort of HIV-1 infected individuals blood serum with ammonium sulfate precipitation method and then and Protein G affinity chromatography. For that 150-200 of patients blood serum was spinned for 20-30 min at 10000 g at 4° C., the pellet was discarded. The supernatant was cooled to 4° C. and stirred slowly in presence of solid ammonium sulfate added to saturation (31.5 g ammonium sulfate per 100 ml of serum=50% saturation at 25° C.). The sediment was clarified at 4000 g for 15 min at 4° C. Supernatant was discarded and the precipitate was dissolved in 20% of the original volume in PBS. Then supernatant in 20 mM PBS pH 7.4 is pored into 1 ml HiTrap protein G column with highly cross-linked 6% agarose from 20 ml syringe. The elution of Ab library from protein G is carried out in 0.1M glycine buffer with pH 2.8 at a flow rate of 0.5 ml\min. The antibody purity 99% was determined by SDS-PAGE.

Polyclonal antibodies were dialized against coupling buffer (0.2 M NaHCO3, 0.5 M NaCl, pH 8.3 overnight at 4° C. The protein mixture (0.5 ml, c=20 mg\ml) was added to 1 ml of NHS-activated Sepharose 4 Fast Flow, previously washed with 10-15 medium volumes of cold 1 mM HCl immediate before use, and incubated overnight at 4° C. After the coupling is completed, any non-reacted groups on the medium should be blocked. Thus, the medium can be kept in 0.5 M ethanolamine, 0.5 M NaCl, pH 8.3 or 0.1 M Tris-HCl, pH 8.5 for a few hours. To wash the medium after coupling, use a method which alternates two different buffers (high and low pH respectively). Buffers used are 0.1 M Tris-HCl buffer pH 8-9 and 0.1 M acetate buffer, 0.5 M NaCl pH 4-5. A suitable procedure could be 3×1 medium volumes Tris buffer followed by 3×1 medium volumes acetate buffer. This cycle is repeated 3-6 times. To prevent microbial contamination antibodies library column can be stored in 20% ethanol.

For capturing the pool of HIV envelop proteins the virus-containing ultracentrifugation precipitate was applied to Ab library column, equilibrated with PBS. One milliliter virus containing sample was diluted up to 10 ml with binding buffer (PBS) and applied to the column at a flow rate of 0.5 ml/min, by recirculation during 2 hours (all buffers were filtered through a 0.22 um). Gradient elution of targeted protein with increased sodium chloride concentration was applied. Two peaks were collected: one—at 23% sodium chloride (0.3M NaCl, major fraction) and second—at 50% sodium chloride (0.6M NaCl, minor fraction). Fractions of 1 ml were collected. The column was re-equilibrated with PBS buffer. Fractions were assayed by measuring absorbance at 214 nm and by Phage Western blot for the detection of viral peptides as it was shown in [7].

The properly glycosylated gp120 molecule consists of 60 kDa polypeptide core (Example 4), and extensive carbohydrate by N-linked glycosylation increases the apparent molecular weight to 90-110 kDa in average and up to 120 kDa. This difference is due to five hyper-variable domains (loops) that contain extensive amino acid substitutions, insertions and deletions for up to 25% overall molecular weight variability. Gp160 polypeptide core is 95-100 kDa (Example 4), and glycosylation increases the molecular weight to 120-160 kDa.

Samples of collected HIV envelop proteins pools were run through reparative SDS-PAGE (FIG. 1), bands of different molecular weight were cut and applied for trypsinolysis.

3. HIV Envelop Proteins Variability Quantitative Mass Spectrometry Analysis

3.1. Triptic Hydrolysis of Polyacrylamide Gel Protein Stripes with Trypsin

Mass spectrometry identification of HIV envelop proteins, especially gp120 in its variability requires 1) the sufficient amount of protein material provided in reverse panning technique cycles; 2) the precise envelop sequence mapping for search and identification of env triptic peptides in samples.

For triptic hydrolysis protein stripes of interest masses from 1D-SDS-PAGE gels stained with Coumassi (FIG. 1A) were cut out and incubated in deionized water at room temperature 15 min, supernatant was discarded. 50-100 μl acetonitryl were added to rinsed gel strips and incubated 20 min at room temperature, acetonitryl excess was evaporated with passive drying. 10 μg/ml modified trypsin (Promega) solution in 0.05M NH₄HCO₃ was added to dehydrated gel stripes and incubated on ice at 4° C. for 60 minutes. Trypsinolysis reaction was run overnight at 37° C. Tryptic peptides extraction from gel was made at room temperature in presence of 0.5% three-fluoric acetic acid in water. Extraction supernatant was used for the following mass spectrometry analysis.

3.2. One-Dimensional Liquid Chromatography-Mass Spectrometry (LC-MS-MS Analysis)

The tryptic peptides from 1D-SDS-PAGE gels were analyzed with ion electrospray quadrupole mass analyzer trap method using HCT-Ultra instrument (Agilent Technologies, USA, cat. #G4240-62001). HCT-Ultra instrument is supplied with integrated liquid chromatography system Agilent Chip Cube™ (Agilent Technologies, USA) consisting of a preliminary enrichment and deionization column (C18), an analytic column (C18), a nanospray needle and quadrupole ion electrospray mass analyzer trap. This instrument is joined to nanochromatography system and sample's fractions collector Agilent 1200 in on line manner.

The mixture of triptic peptides (8 μl in every sample) was delivered into the column for superconcentration and deionization (5 μm Zorbax 300 SB-C18 precolumn, 40 nl, Agilent Technologies) using capillary high pressure pump with stream speed 3 μl/min. in 3% acetonitryl—0.1% formic acid buffer. Chromatography peptides separation in system described above was run using capillary high pressure pump with stream speed 0.3 μl/min. (before splitter) in column with diameter 75 μm×50 μm and length 43 mm, the real elution speed achieved 200 nl/min.

For separation of desalinated triptic peptides the gradient of buffer A (5% acetonitryl, 0.1% formic acid) and buffer B (90% acetonitryl, 0.1% formic acid) was run according to the following program:

| Time, | Buffer B |
|---|---|
| minutes | concentration, % |
| 2 | 0 |
| 5 | 15 |
| 20 | 20 |
| 55 | 50 |
| 60 | 90 |
| 65 | 90 |
| 66 | 0 |

Peptides masses measurements were taken in the quadrupole ion electrospray mass analyzer trap in diapason 300-2200 m/z with trap mass optimization equal to 900; positively charged ions were detected. Ions—predecedors accumulation time in ion trap was 150 ms, the average number of ions in trap (ICC parameter) was 500000, the number of mass-spectrum average was equal 2. Peak's selection for further MS/MS analysis was done in DDA regime, 3 ions-predecedors with maximum intensity were selected and then defragmented during helium co-hits CID regime. Ions with charge number equal 2 or higher and intensity higher than the threshold are taken for tandem experiments. The obtained mass-prints were sent into MASCOT search system.

Peptide's identification confirmation was verified with two software complexes Scaffold 01-07-00 (http://www.proteomesoftware.com) and PEAKS (Canada). The database protein's identification search was run through Los Alamos and NCBI proteomics databases, Peptides with identification expectancy more than 50% were listed in the final schedule. Some results of Scaffold identification are presented in Example 1. However only results obtained with PEAKS analysis (Example 2) allowed quantity-comprehensive HIV envelop triptic peptides identification and gp160 mapping for recombinant cocktail tailoring. PEAKS proteomics makes possible loops V1-V5 ends sequence probability mapping for “playback” HIV strains and can serve the same for HIV isolates analysis.

It is worth to mention that LC-MS-MS mass spectrometry method could not identify HIV gp120 and gp41 triptic peptides in samples which were prepared from the stage of ultracentrifugation virus concentration and without further reverse palming and/or Ab libraries HIV peptides/proteins collection stage.

4. Immunogenic Cocktail Composition Tailoring.

4.1. HIV Envelop Mapping and Original PCR Products Size Analysis.

The same sequence mapping is the basic description of recombinant peptides/proteins cocktail composition being used for immunization of laboratory animals before challenging. There are envelop mapping and sequence analysis calculations that were optional for these two stages of vaccine producing such as 1) gp120 major variants MS-MS identification and 2) recombinant glycosylated env proteins immunogenic composition's production.

gp120 start—V1 loop end about 393 bp
gp120 start—V2 loop end about 534 bp
gp120 start—V3 loop end about 940 bp
gp120 start—V4 loop end about 1193 bp
gp120 start—V5 loop end about 1342 bp
V1 loop start—gp120 end about 1184 bp
V1 loop start—gp41 end about 2240 bp
V2 loop start—gp120 end about 1096 bp
V2 loop start—gp41 end about 2152 bp
V3 loop start—gp120 end about 679 bp
V3 loop start—gp41 end about 1735 bp
V4 loop start—gp120 end about 416 bp
V4 loop start—gp41 end about 1472 bp
V5 loop start—gp120 end about 202 bp
V5 loop start—gp41 end about 1258 bp

The scheme of constant and variable regions in HIV-1 gp120 and primers arrangement is shown in FIG. 26.

For gp120 aminoacid sequence length's possible variations the situation is as follows:

### Mechanic Calculation:

The minimal length of loops V1-V5 sequences is 279 bp or 93 aa
The maximal length of loops V1-V5 sequences is 711 bp or 237 aa
Constant fragments sum length is 1008 bp or 336 aa
Then the whole gp120 sequence length is vary from 1287 to 1719 bp or from 429 to 573 aa

gp41 sequence length does not vary much: 996-1068 bp or 332-356 aa. In average it is 1056 bp (352 aa) for A, C, G and 1035 bp (345 aa) for subtype B.

Then with average gp41 length 1032 bp the total gp160 sequence for subtype A is from 2319 bp to 2751 bp or from 773 aa to 917 aa.

However, mechanic calculations are very much not precise in terms of gp120 sequence and its length analysis due to the evolution selection of real existing protein isoforms.

First, one cannot find only short or only long loops in one gp120 sequence, therefore real minimal and maximal lengths differences are two times smaller.

Second, constant fragments are also variable in length, though it is not that significantly and not in every sequence. Insertions and deletions appear in constant fragments in random regime:

Const 1 length is from 294 to 339 bp (309 in average)
Const 2 length is from 261 to 303 bp (297 in average)
Const 3 length is from 153 to 186 bp (159 in average)
Const 4 length is from 120 to 126 bp (123 in average)
Const 1 length is from 105 to 141 bp (120 in average)

In order to avoid such mistakes in protein molecular weight analysis we suggest it is reasonable to use an alignment calculation. Gaps are mechanically removed from sequences in alignment in Clustal X. Then the software calculates a number of by in every sequence, and range between them becomes obvious.

### Alignment Calculation:

The whole gp120 sequence is vary from 1310 to 1595 bp, or 437-532 aa;

In sequence alignment gp41 sequence length is vary between 1035-1068 bp normally, in average it is 1051 bp or 350 aa for all HIV-1 subtypes;

The whole gp160 sequence therefore is vary from 2361 to 2646 bp, or 787-882 aa.

For peptides and proteins being synthesized in Leishmania tarentolae and further being excreted outside cells into the growth media the signal peptide of L. mexicana 15 bp or 5 aminoacids is added to C-end of protein (this peptide is responsible for transitory expression in growth media). 18 bp 6-His fragment is added to N-terminus for possibility to extract recombinant proteins from growth media using ion-exchange liquid chromatography. So, final recombinant products of HIV env proteins are elongated for 33 bp.

The whole gp120 sequence is vary from 1343 to 1628 bp, or 448-543 aa;

The whole gp160 sequence therefore is vary from 2394 to 2769 bp, or 798-923 aa.

For translating primers into aminoacids sequence standard triplet code is used; aminoacids primer sets can be found easily in presented in Example 3 sequences.

The following primers may be used according to the invention:

### V1 Forward for Subtypes A, G B, C, F1, H

(SEQ ID No. 1)

DNA 5′-CTC TGY GTY ACT TTA XXX XXX XXX-3′

(SEQ ID No. 2)

Protein L C V T L X X X
(X - unknown)

### V2 Forward for all Subtypes:

(SEQ ID No. 3)

DNA 5′-AA AAC TGC TCT TWC AXX XXX XXX X-3′

(SEQ ID No. 4)

Protein N C S Y/F X X X
(W = A or T)

### V3 Forward for A, B, G Subtypes:

(SEQ ID No. 5)

DNA 5′-TA VAA ATT AAT TGT AXX XXX XXX XXX-3′

(SEQ ID No. 6)

Protein K/Q/E I N C X X X X

### V3 Forward for Subtype D:

(SEQ ID No. 7)

DNA 5′-TA VCA ATT AAT TGC AXX XXX XXX XXX-3′

(SEQ ID No. 8)

Protein T/P/A I N C X X X X
(V = A or C or G)

### V4 Forward for all Subtypes:

(SEQ ID No. 9)

DNA 5′-GAA TTT TTC TAT TGY AAX XXX XXX XX-3′

(SEQ ID No. 10)

Protein E F F Y C X X X
(Y = C or T)

### V5 Forward

### For A, B, D, G Subtypes:

(SEQ ID No. 11)

DNA 5′-ACA AGA GAT GGT GGX XXX XXX X-3′

(SEQ ID No. 12)

Protein T R D G X X X

### V5 Forward

### For Subtype C:

(SEQ ID No. 13)

DNA 5′-ACA CGT GAT GGA GGX XXX XXX X-3′

(SEQ ID No. 14)

Protein T R D G X X X

### V5 Reverse for all Subtypes:

(SEQ ID No. 15)

5′-TCC TCC TSC AGG TCT GAA XXX XXX XXX X-3′

### Complementary DNA Strand

(SEQ ID No. 16)

3′-AGG AGG ASG TCC AGA CTT XXX XXX XXX X-5′
(S = G or C)

### V5 Reverse Complement Strand

(SEQ ID No. 17)

DNA 5′-X XXX XXX XXX TTC AGA CCT GSA GGA GGA-
3′

(SEQ ID No. 18)

Protein X X X F R P G/A G G

### V4 Reverse for Subtype G:

(SEQ ID No. 19)

5′-CAA TTT GTT TTA TYY TAC A XX XXX XXX X-3′
(Y = C or T)

### V4 Reverse for Subtypes A, B, C, D:

(SEQ ID No. 20)

5′-TAA TTT GYT TTA TTY TGC A XX XXX XXX X-3′

### V4 Reverse Complement Strand for Subtype G:

(SEQ ID No. 21)

DNA 5′-XXX XXX XXX TGT ARR ATA AAA CAA ATT G-
3′

(SEQ ID No. 22)

Protein X X X C K/R I K Q I
(R = A or G)

### V4 Reverse Complement Strand for Subtypes A, B, C, D:

(SEQ ID No. 23)

DNA 5′-XXX XXX XXX TGC ARA ATA AAR CAA ATT A

(SEQ ID No. 24)

Protein X X X C K/R I K Q I

### V1 Reverse for all Subtypes:

(SEQ ID No. 25)

5′-GCA GTT TTT YAT TTC TYX XXX XXX XXX-3′

### V1 Reverse Complement Strand for all Subtypes:

(SEQ ID No. 26)

DNA 5′-XX XXX XXX XRA GAA ATR AAA AAC TGC-3′

(SEQ ID No. 27)

Protein X X X E I K N C

### V2 Reverse for Subtypes A, D, C:

(SEQ ID No. 28)

5′-AGG TAT TRC AAT TTA TTX XXX XXX X-3′

### For Subtype B:

(SEQ ID No. 29)

5′-CTG AGG TRT TAC AAX XXX XXX X-3′

### For G Subtype:

(SEQ ID No. 30)

5′-AGA CAT TAC AAT TTA TTX XXX XXX X-3′

For subtype F1:

(SEQ ID No. 31)

5′-TTG AGG TAT TRC AAX XXX XXX X-3′

### V2 Reverse Complement Strand for Subtypes A, D, C:

(SEQ ID No. 32)

DNA 5′-XXX XXX XXA ATA AAT TGY AAT ACC T-3′

(SEQ ID No. 33)

Protein X X X I N C N T

### V2 Reverse Complement Strand for Subtype B:

(SEQ ID No. 34)

DNA 5′-X XXX XXX XXT TGT AAY ACC TCA G-3′

(SEQ ID No. 35)

Protein X X X C N T S

### V2 Reverse Complement Strand for G Subtype:

(SEQ ID No. 36)

DNA 5′-XXX XXX XXA ATA AAT TGT AAT GTC T-3′

(SEQ ID No. 37)

Protein X X X I N C N V

### V2 Reverse Complement Strand for Subtype F1:

(SEQ ID No. 38)

DNA 5′-X XXX XXX XXT TGY AAT ACC TCA A-3′

(SEQ ID No. 39)

Protein X X X C N T S

### V3 Reverse

For subtypes A, D, (C):

(SEQ ID No. 40)

5′-AAA GTT TBA TTC CAX XXX XXX XX-3′

### For Subtype B:

(SEQ ID No. 41)

5′-AAA GTG TTR TTC CAX XXX XXX XX-3′

### V3 Reverse Complement Strand for Subtypes A, D, (C):

(SEQ ID No. 42)

DNA 5′-XXX XXX XXX TGG AAT VAA ACT TT

(SEQ ID No. 43)

Protein X X X W N K/Q/E T

### V3 Reverse Complement Strand for Subtype B:

(SEQ ID No. 44)

DNA 5′-XXX XXX XXX TGG AAY AAC ACT TT

(SEQ ID No. 45)

Protein X X X W N N T

gp41(160) Reverse for A, B, D Subtypes NotI Restriction Site is Marked Light Grey, 6His Tag is Marked Dark Grey)

(SEQ ID No. 46)

5′-ATA GCG GCC GCC TAG TGG TGG TGA TGG TGG TGT AG
YAA AGC YCT TTC NAA GCC CTG TC-3′

gp41(160) Reverse Complement Strand for A, B, D Subtypes

(SEQ ID No. 47)
5′ - GA CAG GGC TTN GAA AGR GCT TTR CTA CAC CAC CAT CAC CAC CAC TAG
GCG GCC GC TAT
(SEQ ID No. 48)
Protein Q G X E R A L L H H H H H H stop

gp41(160) Reverse for Subtype A (Rare Variant)

(SEQ ID No. 66)

ATA GCG GCC GCC TAG TGG TGG TGA TGG TGG TGT AGC
AAA GCY CTT TCN GCG CCC TGT C

gp41(160) Reverse for C Subtype

(SEQ ID No. 67)

ATA GCG GCC GCC TAG TGG TGG TGA TGG TGG TGT WGC
AAA GCT GCT TCA AAG CCC TGT C

gp41(160) Reverse for G Subtype

((SEQ ID No. 68)

ATA GCG GCC GCC TAG TGG TGG TGA TGG TGG TGT AGC
AAA GCY CTT TCN AAG CCT TGT C

gp120 Reverse Const5 for all Subtypes (NotI Restriction Site is Marked Light Grey, 6His Tag is Marked Dark Grey)

(SEQ ID No. 49)

5′-ATA GCG GCC GCC TAG TGG TGG TGA TGG TGG TGT CTT
TTT TCT CTY TSC ACC ACT CTY CT-3′

gp120 Reverse Complement Strand Const5 for all Subtypes

(SEQ ID No. 50)
5′AGR AGA GTG GTG SAR AGA GAA AAA AGA CAC CAC CAT CAC CAC CAC
TAG GCG GCC GCT AT
(SEQ ID No. 51)
R R V V E/Q R E K R H H H H H H stop

Forward gp120 Const1 for A Subtype with XbaI Site on 5′ End (Marked Grey)

(SEQ ID No. 52)
DNA 5′-A ATT CTA GAC RCT RCA GAA AAC TTG TGG GTY AC-3′
(SEQ ID No. 53)
Protein L D T/A T/A E N L W V LDXXENLWV

Forward gp120 Const1 for B Subtype (with XbaI Site on 5′ End)

(SEQ ID No. 69)

5′-AAT TCT AGA CGC TRC AGA AMA ATT GTG GGT CAC-3′

Forward gp120 Const1 for C Subtype (with XbaI Site on 5′ End)

(SEQ ID No. 70)

5′-AAT TCT AGA CGT RRT GGG RAA CTT GTG GGT CAC-3′

Forward gp120 Const1 for G Subtype (with XbaI Site on 5′ End)

(SEQ ID No. 71)

5′-AAT TCT AGA CGC CTC ARA TAA CTT GTG GGT CAC
AG-3′

In a preferred embodiment, following primers are used:
(i) Forward gp120 Const1 for A subtype (with XbaI site on 5′ end)
   5′-AAT TCT AGA CRC TRC AGA AAA CTT GTG GGT YAC-3′ (SEQ ID No. 52), or a derivative thereof lacking the XbaI site, and/or comprising a restriction site at the 5′ end
(ii) Forward gp120 Const1 for B subtype (with XbaI site on 5′ end)
   5′-AAT TCT AGA CGC TRC AGA AMA ATT GTG GGT CAC-3′ (SEQ ID No. 69), or a derivative thereof lacking the XbaI site, and/or comprising a restriction site at the 5′ end
(iii) Forward gp120 Const1 for C subtype (with XbaI site on 5′ end)
   5′-AAT TCT AGA CGT RRT GGG RAA CTT GTG GGT CAC-3′ (SEQ ID No. 70), or a derivative thereof lacking the XbaI site, and/or comprising a restriction site at the 5′ end,
(iv) Forward gp120 Const1 for G subtype (with XbaI site on 5′ end)
   5′-AAT TCT AGA CGC CTC ARA TAA CTT GTG GGT CAC AG-3′(SEQ ID No. 71), or a derivative thereof lacking the XbaI site, and/or comprising a restriction site at the 5′ end,
(v) V1 reverse for all subtypes:

(SEQ ID No. 25)

5′-GCA GTT TTT YAT TTC TYX XXX XXX XXX-3′

(vi) V2 reverse for subtypes A, D, C:

(SEQ ID No. 28)

5′-AGG TAT TRC AAT TTA TTX XXX XXX X-3′

(viii) V2 reverse for subtype B:

(SEQ ID No. 29)

5′-CTG AGG TRT TAC AAX XXX XXX X-3′

(ix) V2 reverse for subtype G:

(SEQ ID No. 30)

5′-AGA CAT TAC AAT TTA TTX XXX XXX X-3′

(x) V2 reverse for subtype F1:

(SEQ ID No. 31)

5′-TTG AGG TAT TRC AAX XXX XXX X-3′

(xi) V3 reverse for subtypes A, D, (C):

(SEQ ID No. 40

5′-AAA GTT TBA TTC CAX XXX XXX XX-3′

(xii) V3 reverse for subtype B:

(SEQ ID No. 41)

5′-AAA GTG TTR TTC CAX XXX XXX XX-3′

(xiii) V4 reverse for subtype G:

(SEQ ID No. 19)

5′-CAA TTT GTT TTA TYY TAC A XX XXX XXX X-3′

(xiv) V4 reverse for subtypes A, B, C, D:

(SEQ ID No. 20)

5′-TAA TTT GYT TTA TTY TGC A XX XXX XXX X-3′

(xv) V5 reverse for all subtypes:

(SEQ ID No. 15)

5′-TCC TCC TSC AGG TCT GAA XXX XXX XXX X-3′,

and
(xvi) gp41(160) Reverse for A, B, D subtypes, including sequence encoding His-Tag:

(SEQ ID No. 46)

ATA GCG GCC GCC TAG TGG TGG TGA TGG TGG TGT AG YAA
AGC YCT TTC NAA GCC CTG TC

(xvii) a primer derivative of (viii) lacking the sequence encoding a His-Tag, and/or comprising a sequence encoding a tag,
(xviii) gp41(160) Reverse for subtype A (rare variant)

(SEQ ID No. 66)

ATA GCG GCC GCC TAG TGG TGG TGA TGG TGG TGT AGC
AAA GCY CTT TCN GCG CCC TGT C

(xix) gp41(160) Reverse for C subtype

(SEQ ID No. 67)

ATA GCG GCC GCC TAG TGG TGG TGA TGG TGG TGT WGC
AAA GCT GCT TCA AAG CCC TGT C,

and
(xx) gp41(160) Reverse for G subtype

(SEQ ID No. 68)

ATA GCG GCC GCC TAG TGG TGG TGA TGG TGG TGT AGC
AAA GCY CTT TCN AAG CCT TGT C.

In a further preferred embodiment, following primers are used:
(i) V1 forward for subtypes A, G B, C, F1, H:

(SEQ ID No. 1)

5′-CTC TGY GTY ACT TTA XXX XXX XXX-3′

(ii) V2 forward for all subtypes:

(SEQ ID No. 3)

5′-AAA ACT GCT CTT WCA XXX XXX XXX-3′

(iii) V3 forward for A, B, G subtypes:

(SEQ ID No. 5)

5′-TAV AAA TTA ATT GTA XXX XXX XXX X-3′

(iv) V3 forward For subtype D:

(SEQ ID No. 7)

5′-TAV CAA TTA ATT GCA XXX XXX XXX X-3′

(v) V4 forward for all subtypes:

(SEQ ID No. 9)

5′-GAA TTT TTC TAT TGY AAXXX XXX XXX-3′

(vi) V5 forward
   For A, B, D, G subtypes:

(SEQ ID No. 11)

5′-ACA AGA GAT GGT GGX XXX XXX X-3′

(vii) V5 forward For subtype C:

(SEQ ID No. 13)

5′-ACA CGT GAT GGA GGX XXX XXX X-3′

(viii) gp41(160) Reverse for A, B, D subtypes, including sequence encoding His-Tag:

(SEQ ID No. 46)

ATA GCG GCC GCC TAG TGG TGG TGA TGG TGG TGT AG
YAA AGC YCT TTC NAA GCC CTG TC

(ix) a primer derivative of (viii) lacking the sequence encoding a His-Tag, and/or comprising a sequence encoding a tag,
(x) gp41(160) Reverse for subtype A (rare variant)

(SEQ ID No. 66)

ATA GCG GCC GCC TAG TGG TGG TGA TGG TGG TGT AGC
AAA GCY CTT TCN GCG CCC TGT C

(xi) gp41(160) Reverse for C subtype

(SEQ ID No. 67)

ATA GCG GCC GCC TAG TGG TGG TGA TGG TGG TGT WGC
AAA GCT GCT TCA AAG CCC TGT C

(xii) gp41(160) Reverse for G subtype

(SEQ ID No. 68)

ATA GCG GCC GCC TAG TGG TGG TGA TGG TGG TGT AGC
AAA GCY CTT TCN AAG CCT TGT C

(xiii) gp120 Reverse Const5 for all subtypes, including sequence encoding His-Tag

(SEQ ID No. 52)

5′-ATA GCG GCC GCC TAG TGG TGG TGA TGG TGG TGT
CTT TTT TCT CTY TSC ACC ACT CTY CT-3′,

and
(xiiii) a primer derivative of (viii) lacking the sequence encoding a His-Tag, and/or comprising a sequence encoding a tag.

The complete HIV envelop proteins mappings for primers design are presented in the Example 3.

4.2. Glycosylated Envelop Proteins Pool Expression and Separation.

An inducible expression of HIV envelop proteins from amplified genetic material is carrying out in Leishmania tarentolae system as it was previously described in [7] with the following improvements. The cloning of mapped amplified genes, transformation and further antibiotics selection, is being run as a pool cloning, i.e. vectors with amplified genes of envelop proteins from biomaterial with approximately equal lengths and variable sequences are used as a mixture for transformation with electroporation method. Cuvette electro squid impulse instrument BTX-830 with HV module (Harvard Instruments), 450V twice in 3 milliseconds is being run for the task. Transformed L. tarentolae variants are exposed to G418 selection in liquid media in horizontal positioned flasks at 26° C. and light stirring. Monoclones are not being selected on solid media not to lose their natural variability of envelop proteins.

Large scale tetracycline-inducible expression is carried out into cultivation media. It is run in hollofiber system in order to provide the possibility for eukaryotic proteins effective isolation in reasonable quantities from the media freed from large concentrations of proteins and peptides, animal-originated proteins as well.

4.3. Glycosylated Recombinant Proteins Molecular Weight Calculations

For aminoacid's sequence molecular weight determination the Free Protein and Peptide's Molecular Weight Calculator software was used: http://www.encorbio.com/protocols/Prot-MW.htm. Several examples are presented below and in Example 4 calculations.

160 #1 (B.FR.83.HXB2_LAI_IIIB_BRU_K034) There are 856 Amino acids, and MW is 97241.93 Daltons, or 97.24 KiloDaltons; Charge is 30 assuming N and C termini are not blocked

120 #1 There are 511 Amino acids, and MW is 57695.64 Daltons, or 57.70 KiloDaltons

41#1 There are 345 Amino acids, and MW is 39564.30 Daltons, or 39.56 KiloDaltons

160 #43 (K.CM.96.MP535_AJ249239): There are 842 Amino acids, and MW is 95252.58 Daltons, or 95.25 KiloDaltons, Charge is 22 assuming N and C termini are not blocked.

120 #43: There are 497 Amino acids, and MW is 55884.46 Daltons, or 55.88 KiloDaltons

41 #43: There are 345 Amino acids, and MW is 39386.14 Daltons, or 39.39 KiloDaltons

160 #6 (our ALRU.03.03RU20_{—}06_{—}13_AY500393) There are 805 Amino acids, and MW is 90669.32 Daltons, or 90.67 KiloDaltons: Charge is 24 assuming N and C termini are not blocked

120 #6 There are 493 Amino acids, and MW is 55328.29 Daltons, or 55.33 KiloDaltons

41#1 There are 346 Amino acids, and MW is 39333.71 Daltons, or 39.33 KiloDaltons

160 #26 (C.ZA.01.01ZATM45_AY228557) There are 878 Amino acids, and MW is 99122.18 Daltons, or 99.12 KiloDaltons; Charge is 15.5 assuming N and C termini are not blocked

120 #26 (C.ZA.01.01ZATM45_AY228557) There are 526 Amino acids, and MW is 59182.32 Daltons, or 59.18 KiloDaltons

41 #26 (C.ZA.01.01ZATM45_AY228557) There are 352 Amino acids, and MW is 39957.88 Daltons, or 39.96 KiloDaltons

5. Immunogenic Composition Efficiency Analysis

5.1. Techniques for Immunocompetent PBMC, DC and Cell Cultures Obtaining for Background SCID Mice Engraftment

Techniques for engrafting SCID mice with human immunocompetent cells are known in the art. Normally the immunocompetent cells are selected from bloodstream-originated group of PBMC, dendrite cells, B-lymphocytes and T-lymphocytes. However it occurred that non of known and described methods are able to provide Hu-SCID mice for HIV vaccine efficiency testing. As discussed above, for the evaluation of an HIV vaccine infection-preventive potential the Hu-SCID-mouse should be able to show two reactions. First, mouse should emit a human-type HIV-specific immune response after being inoculated with the HIV vaccine. Second, the same animal without vaccination should be able to catch and to develop HI-virus challenging in means of viral load detectable in its bloodstream exactly like HIV infection presence is being determined in humans.

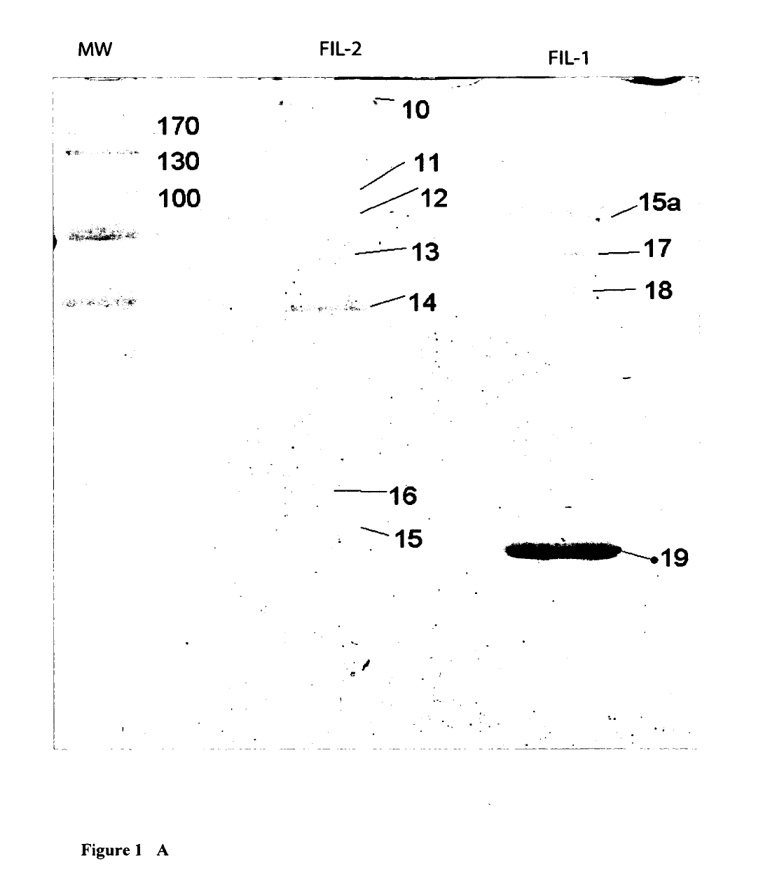

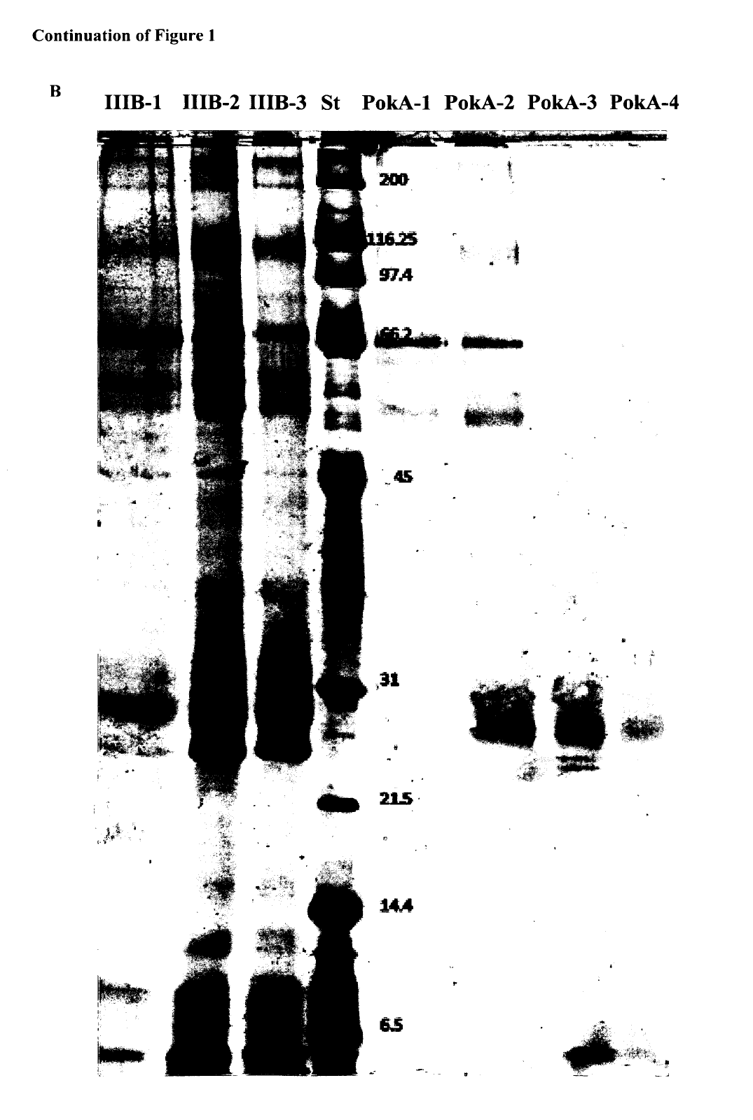

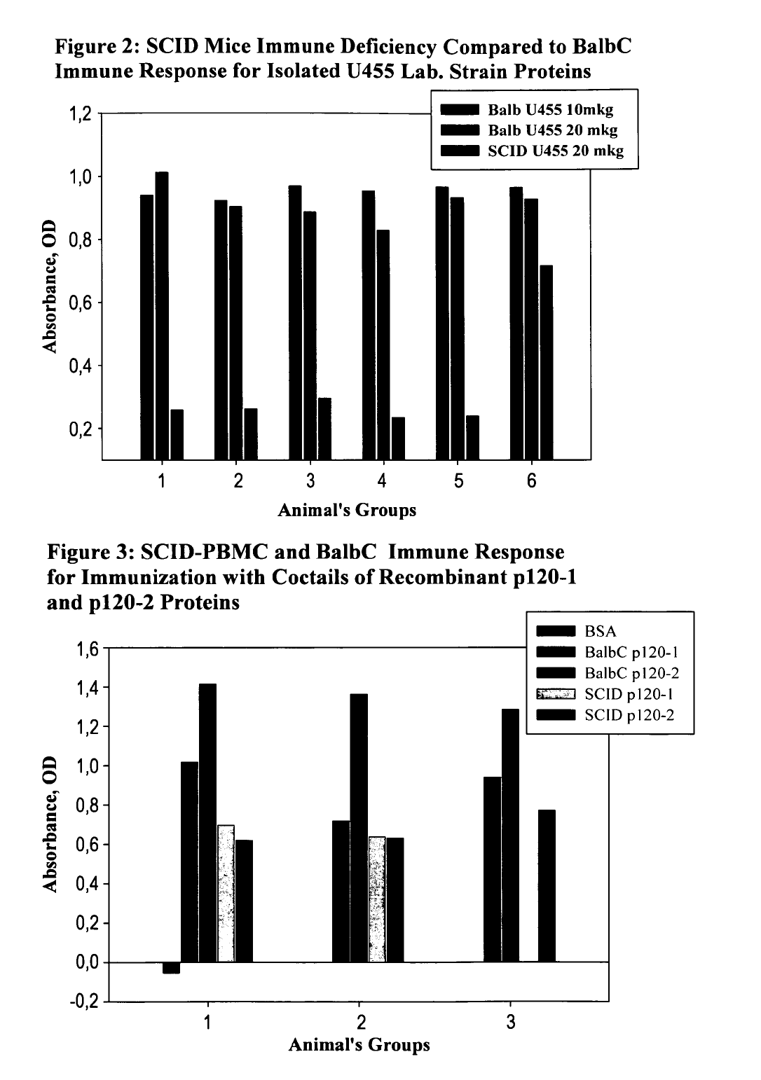

Maintaining and experimental work with immune deficient animals are complicated with a number of special conditions and restrictions, however these expensive in vivo systems are worth the troubles they require for the survivorship. Thus, when new medicines are being tested for toxicity in vivo, SCID mice are known to deliver dosage regimes the most close to human's, while normal laboratory mice, rats and dogs ID₅₀ are 10-2 times higher and nude mice—2-5 times lower than human ones. However to achieve informative results the researchers have to consider this number of conditions and adopt them for animals handling to avoid obtaining some false-positive data. The first little example of this approach is FIG. 2 presenting how SCID's immune status was checked before experiments with vaccines. SCID and BalbC mice were immunized with concentrated in 20% sucrose gradient ultracentrifugation U455 proteins mixture and ELISA results were compared with BalbC mice immune response. One of six SCIDs accidentally provided low immunity this time (FIG. 2). In case SCID mice show some response for immunization (and it might happen for some percent of baby mice to be born with it) it is necessary to remove these animals from experiment assessment because viral infection in these mice might be blocked with this even low immunity and not with vaccination. For reproducing animals without such defect 3-5 cycles of brother-sister inbreeding is recommended.

For measuring the efficacy of a vaccine, all methods generally known in the art for that purpose may be used. In particular, the efficacy of the HIV vaccine may be determined by determining the viral load in a body fluid sample of the mouse. Said body fluid sample may be blood serum.

For determining the viral load in animal's bloodstream every method known in the art can be used. This includes Real Time PCR, RT-PCR and p24 ELISA.

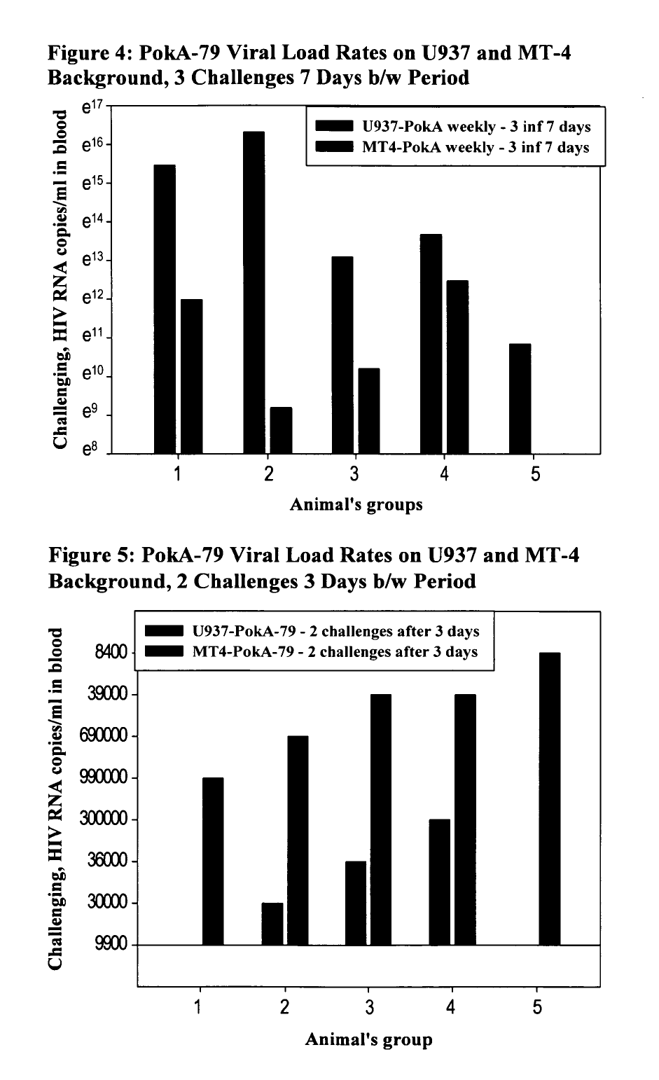

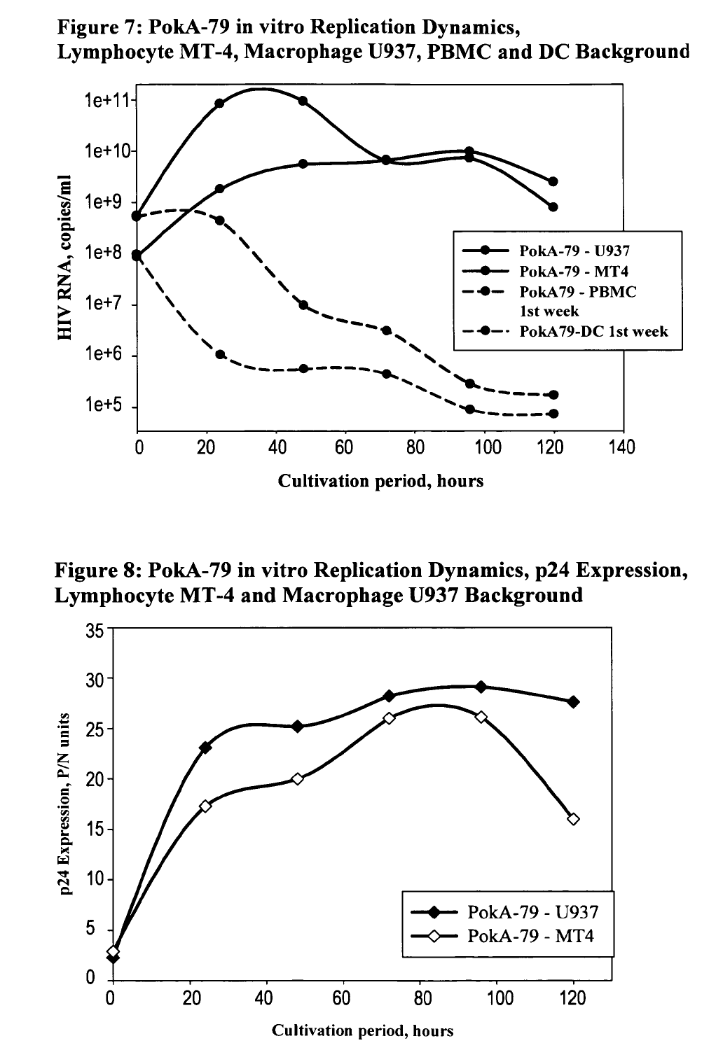

Our two-years long in vivo experiments with Hu-SCID mice proved that, contradictory to well-admitted opinion, neither fresh PBMC (PBL) along, nor dendrite cells, nor fresh patient's HIV isolates can provide HIV challenging as a viral load in animals blood serum (see FIGS. 7, 18). However PBMC (PBL) and dendrite cells introduced into SCID mice can elicit HIV immune response which strength and specificity depend on immunocompetent cells characteristics, regimes of cells engraftment and also on immunogenic composition efficient quality (FIGS. 2-3). Contrariwise, T-, B-based immortalized or tumor cell cultures being used for in vitro cultivation of HIV laboratory strains are able to deliver detectable viral loads in Hu-SCID mice bloodstream (FIGS. 4-6, 13, 16) but they cannot provide the HIV-specific immune response after administration of a vaccine. The task for presented research was to make these two functions available in the same Hu-SCID mouse.

Introduction of cells of human origin into the SCID mice may be achieved by using any conventional route, such as via the intraperitoneal route. The intraperitoneal route is normally preferred due to the ease of administration and allowing administration of a volume of fluid. The cells may be introduced in form of a suspension in any suitable medium, allowing cell survival, such as phosphate buffered saline (PBS) or Dulbecco's modified Eagle's medium (DMEM), Roswell Park Memorial Institute medium (RPMI) or Glasgow's Minimal Essential Medium (GMEM).

Preferably the immunocompetent cells are cultivated in vitro prior to being introduced into the SCID mice because their engraftment efficiency can be enhanced with cytokines stimulators. Preferred stimulators include but are not limited to interleukin factors such as IL-2 (interleukin-2), IL-4 (interleukin-4), IL-12 (interleukin-12), and other factors such as phitohemagglutinin, INF-γ and GCSF (granulocyte colony stimulating factor). The immune stimulators promote growth and/or development of e.g. T, B, and hematopoietic cells.

Healthy donor peripheral blood mononuclears (PBMC) are isolated by standard Ficoll gradient blood separation method with the following adjustments. Blood collected in 10 ml vacutainers in presence of EDTA and 30 ME/ml heparin is being left at room temperature for 1.5-2.5 hours in vertical position until serum with lymphocyte's fraction is in upper layer, and erythrocytes, platelets with fibrinous blood proteins are in lower layer. Then blood serum is collected into 50 ml sterile tubes and centrifuged at 1200 g for 20-25 min. The pellet—mostly big PBMC cells—is collected and put into culture flasks for cultivation, the supernatant—human blood serum (HBS)—is stored at −20° C. in 15 ml tubes for further PBMC in vitro cultivation 1-2% supplementation. The lower layer donor blood is diluted 4-5 times with DPBS (Dulbecco's modified phosphate buffer saline) and pored over equal volume of 1.077 g/ml Ficoll into 15 ml sterile tubes. 980 g gradient centrifugation for 30 min. left small differentiated lymphocytes fraction and a few mononuclears in interphase. Interphase cells from the circle are collected and rinsed in RPMI-1640 twice with spinning for 10 min. at 980 g.

It is possible to make SCID mice engraftment with freshly isolated PBMC. But it is more reasonable to make 3-days in vitro cultivation before. If necessary one donor PBMC can be cultivated in vitro for 2-4 weeks.

The obtained cell suspension is diluted in 15-20% FBS-HBS-supplied culture medium to 2×10⁶ cells in 1 ml and incubated in Petri dishes or 6-well culture plates at 37° C. and 5% CO₂. In case cells have to be divided into lymphocytes and monocytes sub-populations cell suspension is being layered onto dishes or culture flasks plastic previously treated with human blood serum (HBS). After 1-2 hours incubation at 37° C. non-adhesion fraction (lymphocytes) are transferred to a new culture dishes, and ancored monocytes (DC-producers) are supplied with new medium for cultivation. Standard scheme is used for cells proliferation activation. Mitogenic lectin—phitohemagglutinin (PHG) in sub-mitogenic concentration 1-2 μg/ml is being added during the 1^{st} day of incubation. 24 hours later 10 μg/ml exogenous cytokine interleukine-2 is added to culture medium. This activation scheme is effective and allows diminishing activator's working concentration. PHG is getting into interaction with T-cell receptors complex and induces cell cycle G₀-G₁ phase transfer. Cells in G1 phase cells are more sensitive to IL-2 effect.

22 collections of blood of “one donor” were carried out in 2009. 13 blood collections of “one donor” and 4 collections of blood from different donors were carried out in 2010. 4 blood collections of “one donor” were made in 2011.

Every blood samples collection from the same donor delivers from 80 to 200 million freshly isolated PBMC. The cell count for each introduction of cells of human origin may vary from 1×10⁶ to 5×10⁷ cells per introduction, preferably from 1×10⁷ to 1.5×10⁷ cells per introduction. The introduction of cells of human origin may be repeated weekly, or twice weekly, for running one experiment every Hu-SCID mouse receives from 4 to 10 immunocompetent cells introduction depending on the task of a current assay: immunization effect boost, challenging or consecutive combination of both for vaccination to prevent challenging development.

In case PBMCs are introduced into SCID mice followed by immunization these mice are referred to as “PBMC-Hu-SCID mice”. In case dendrite cells (DCs) are introduced into SCID mice followed by immunization and subsequent introduction of cells of the immunogenic background such as PBMCs, these mice are referred to as “DC-PBMC-Hu-SCID mice”.

The dendrite cells may have been obtained by cultivation of human PBMC in the presence of cytokines capable of inducing the formation of dendrite cells in vitro. Methods for the production of dendrite cells in vivo are known in the art (see e.g. [25]).

Thus, 3-days in vitro pre-cultivated PBMC, freshly isolated PBMC and DC can be used for SCID mice “humanization” as immune response formation background. 4-5 weeks old SCID mice were inoculated with re-suspended in PBS (phosphate buffer saline) or DMEM without phenol red (Dulbecco's modified Eagle's media) 5-15×10⁶ PBMC intraperitoneally once a week two times. Two weeks later the 3^{rd} PBMC inoculation was performed and next day mice were immunized subcutaneously with peptides mixture obtained as it was described previously in [7] and in the current application. Each animal received 25 mkg peptides in cocktail mixed in 200 μl incomplete Freund adjuvant for 1 hour at +4 C. Immunizations were repeated three times after two weeks, SCID mice were provided with fresh portions of PBMC weekly. The special requirement was the supplying one donor PBMC 5-15×10⁶ cells for each inoculation for one mouse (one donor's isolation was enough for a group of 5-10 animals). Immune response was tested in ELISA assays. Two weeks after the last immunization SCID mice pre-treated with PBMC weekly had U455, p120-1 and p120-2 immunity rates the intensity comparable with response of normal BalbC mice (FIGS. 2-3).

The other method of immune response formation in SCID mice we used included dendrite cells cultivation and initial mice supply. Donor PBMC were isolated with described above method and cultivated in vitro for 6 weeks in presence of differentiation blocking cytokine's factors—IL-2 (interleukin-2), IL-4 (interleukin-4) and GCSF (granulocyte colony stimulating factor) until cells are multiplied in number several times. 4-5 weeks SCID mice were inoculated with re-suspended in PBS or DMEM without phenol red 15×10⁶ dendrite cells. Then after two weeks animals were immunized subcutaneously with p120-1 and p120-2 peptides mixture. Each animal received 25 mkg peptides in cocktail mixed in 200 mkl incomplete Freund adjuvant for 1 hour at +4 C. Immunization was repeated three times after two weeks, SCID mice were supplied with fresh portions of PBMC once in 2-3 weeks. PBMC for next inoculations were isolated from the blood of the same donor whose blood was used as a source for dendrite cells cultivation for the current group of animals used in experiment. Response for immunization was of much weak intensity than pre-treated with PBMC SCID mice showed, but it was more elongated, and it didn't disappear completely after two weeks passed since the last immunization.

The conditions important for HIV-specific short-time immune boost are summarized in Table 1. There are relatively small number of PBMC—5-15×10⁶ cells for one animal for a single inoculation (1), the presence of PBMC (dendrite) cells substrate one-two weeks before every immunization (2), the implementation of one donor material for one mouse (group of mice) preventing them from GVHD development and early death (3). It is detectable also that intensity (specificity and values) of response after immunization/vaccination is 2-4 times higher in case one donor PBMC material is being used; immune response activity is comparable to that non-immune deficient mice elicit as murine antibodies production for the same immunization/vaccination (3). Several immunizations, preferably three, should be done during one vaccination course (4).

The quality of biomaterial used for vaccination is crucial for success of vaccinations. Immunogenic cocktails of recombinant HIV envelop peptides/proteins with sufficient not less than 20 mkg target peptide's concentrations for one animal should be used for successful vaccination (5). In case low-immunogenic and low-specific compositions such as HIV DNA vaccines are used Hu-SCID model will not provide positive results (6). It is preferable to use adjuvants for immune boost compositions in course of immunization/vaccination (Table 1). The limited period of detectable HIV-specific immune response (7-8) 3-4 weeks for PBMC and 5-6 weeks for DC-PBMC mice is due to: 1) animals were immunized subcutaneously with simple peptide's cocktails without immune boost composition; 2) small-animal models lifespan is months, not years.

The composition/cocktail for successful immunizations against HIV challenging normally contains a great number of envelop sequences from decades for HIV laboratory strains to a library of hundreds and thousands for HIV-infected patient's isolates cloned as a pool in suitable expression system vector (See “Envelop variability analysis” pp. 11-13).

TABLE 1
HIV-specific immune boost conditions in PBMC-SCID and DC-PBMC-SCID mice.

| | | Immune Response for | |
|---|---|---|---|
| | | p120 when Treatment is: | |
| | Applied Treatment (Conditions) for Hu-SCID Mice Immunity | | Not |
| ## | for HIV env Peptides/Proteins | Applied | Applied |
| 1 | Modest PBMC, DC/PBMC number, 5-15 × 10⁶ for one animal's | + | − |
| | each dose | | |
| 2 | Substrate (PBMC, DC) cells providing 1-2 weeks before | + | − |
| | immunizations | | |
| 3 | One donor PBMC material for each mouse during | + | − |
| | immunizations course | | |
| 4 | Three or more immunizations for the course with 2 weeks | + | −+ |
| | period before the next one for each PBMC-SCID, DC-PBMC- | | |
| | SCID mouse | | |
| 5 | Vaccination/immunization with immunogenic HIV-env-specific | + | − |
| | components such as cocktails/mixtures of recombinant viral | | |
| | envelop peptides/proteins | | |
| 6 | Vaccination/immunization with HIV genes-containing viral | − | − |
| | vectors and non-viral plasmid DNA | | |
| 7 | Detectable env-specific immune boost time period for PBMC- | 2-4 weeks | − |
| | Hu-SCID mice after the last immunization | | |
| 8 | Detectable env-specific immune boost time period for DC- | 3-4 weeks | − |
| | PBMC-Hu-SCID mice after the last immunization | | |

Immunization may be carried out once or in a number of subsequent steps, e.g. it may be repeated after some time, e.g. after one to three weeks, preferably after two weeks two to four times, preferably three times.

The Hu-SCID-mouse may be vaccinated with the HIV immunogenic composition 2 weeks after the first engrafting of the human immunocompetent cells. The preferred route for administering the vaccine to the mice is via subcutaneous injection though intranasal route is also possible and could be preferred for bigger immune deficient animals. The vaccine to be tested may be administered to the Hu-SCID mice three or more times (three to six times). As the vaccine is a peptide or protein mixture, the amount of vaccine peptides or proteins administered to the Hu-SCID mouse is between 15 and 50 μg in each step for each mouse. Immunizations of the animals are performed once in 2 weeks and repeated 3 or more times.

Further Experiments were Targeted to Test HIV Laboratory Strains Challenging Maintenance in Hu-SCID Mice.

The conditions, important for virus challenging formation in vivo are summarized in Table 3. There are two possible methods of infection modeling in SCID mice different in virus replication substrate:
1) donor PBMC or DC as background;
2) in vitro cultivation cell culture as background.

The first several experiments challenging of the hu-SCID mice with an amount of HIV laboratory strain infectious doses in human PBMC culture, e.g. from about 5×10⁴ to 5×10⁶ TCID₅₀ (50% Tissue Culture Infective Dose) for one animal have been ended with negative results. In order to make out what is getting wrong in regimes of challenging it was reasonable to run a set of cell culture background experiments HIV laboratory strain as a positive control.

According to the cell culture background method 5-8 weeks old SCID mice were inoculated intraperitoneally with 5-10×10⁶ MT-4 or U937 pure culture cells rinsed twice in PBS from cultivation serum with 10 min spinning at 800 rpm and re-suspended in PBS. One week later pure cell culture inoculation was repeated. The next day after the second MT-4 or U937 cells inoculation animals were challenged intraperitoneally with 5×10³-10⁴ TCID₅₀ one of HIV-1 laboratory strains infectious doses in cell culture MT-4 or 5×10²-5×10³ TCID₅₀ infectious doses in U937. Challenges were repeated 2-3 times with different time periods between, SCID mice were provided with fresh portions of MT-4 or U937 weekly. The other way of challenging allows simultaneous inoculation of Hu-SCID mice with 10×10⁶ pure MT-4 or U937 culture and 5×10⁶ MT-4 or U937 infected with one of HIV-1 laboratory strains mixed in one syringe every 3-7 days, respectively. In both methods applied blood samples were collected from the animals 1-4 weeks after the last challenging and tested for viral infection rates by Real Time PCR, they achieved 10³-10⁸ HIV RNA copies per ml in bloodstream (FIGS. 4, 5, 6).

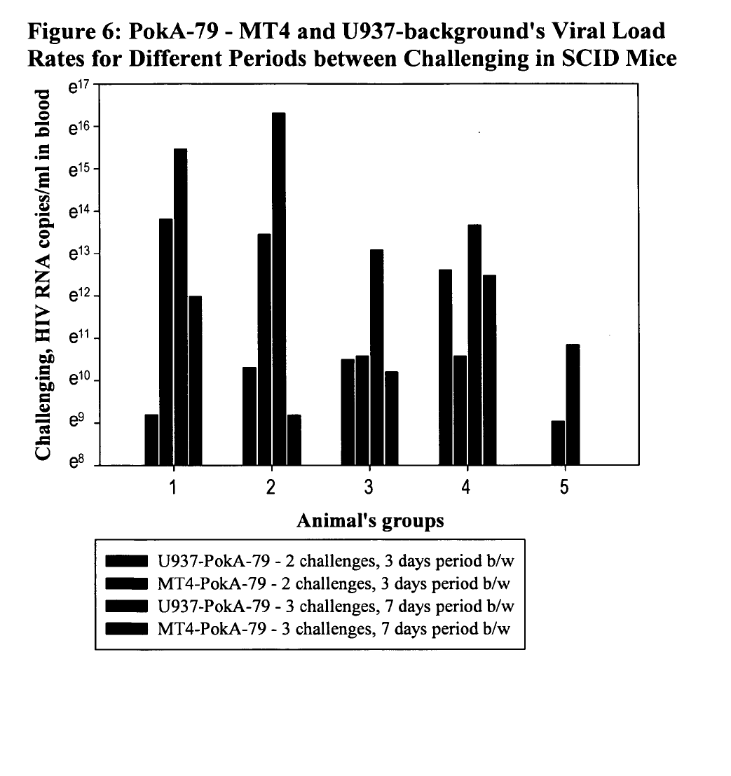

HIV laboratory strains can have aggressive or slow multiplication kinetics; quickly developing strains are better and more demonstrative for in vivo experiments. We have used only highly aggressive strains of A and B subtypes with fast replication dynamics and high rates of viral concentration in vitro—10¹⁰-10¹² copies/ml HIV RNA within 1-4 days of cultivation (Table 2). But cell culture virus replication kinetics has the same value for in vivo viral load and percentage of successful challenging. Analysis of viral load in SCID-PBMC mice bloodstream after 2-4 times challenging with the same laboratory strain cultivated on different cell cultures shows dependency of HIV RNA copies rate from the regime of inoculation (FIG. 4-6). When inoculations of infected with laboratory virus cells were made 3 days after the previous ones rates of viral load (HIV RNA copies per one ml of mice blood number) were always several times higher for variant which was cultivated on MT-4 culture background (FIG. 4) than for the variant cultivated on monocyte's U937 background. When inoculations were made 7 days after the previous ones HIV RNA copies numbers were always several times higher for cultivated on U937 background variant (FIG. 5) than for the variant cultivated on lymphocyte's MT-4 background. In one experiment viral load in SCID mice bloodstream was also 3-10 times higher for MT-4 cultivation with 3 days period between challenging than for U937 cultivation and 3-15 times higher for U937 cultivation with 7 days period between challenging than for MT-4 cultivation (FIG. 6). The same in vivo HIV infection development dynamics has been observed for U455 and H9/IIIB laboratory strains (FIG. 13).

In vivo HIV-1 laboratory strains cultivation dynamics on lymphocyte's and monocyte's background repeats precisely in vitro dynamics of virus strains multiplication. So, in vitro replication titre for U455, PokA-79 and H9/IIIB measured both as Real Time HIV RNA copies data and p24 expression ELISA results were reaching maximums in 24-48 hours for lymphocyte MT-4 and MT-2 cell cultures (Table 2, FIGS. 7-8, 14-15). Contrariwise, in vitro replication titre for U455, PokA-79 and H9/IIIB measured both as RNA copies and p24 expression were growing to maximums within 72-96 hours for monocyte U937 cell culture (Table 2, FIGS. 7-8, 14-15). As it is necessary to use log phase infected cell culture for in vivo challenging success we can conclude 3 days period between challenging is the best for the achievement of the highest viral load numbers in lymphocyte culture background and 7 days period—for the highest viral load in monocyte culture background.

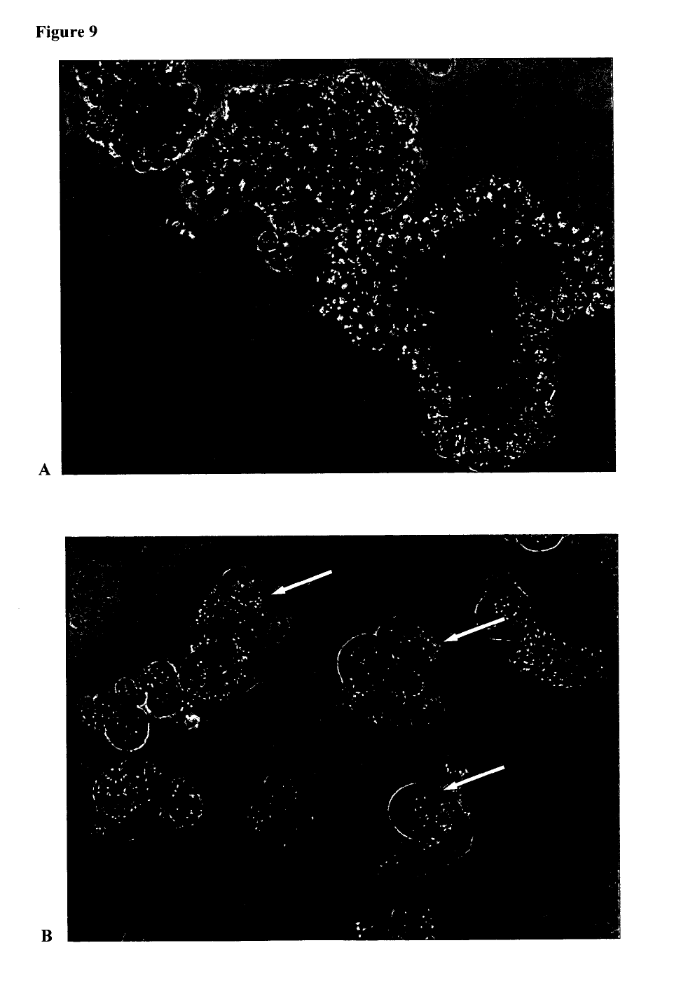

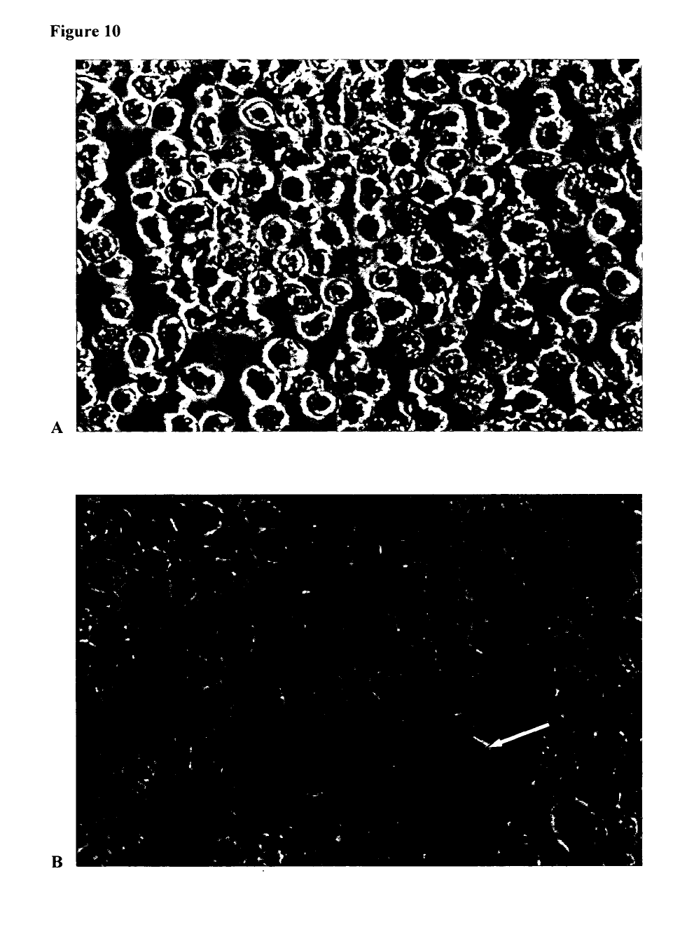

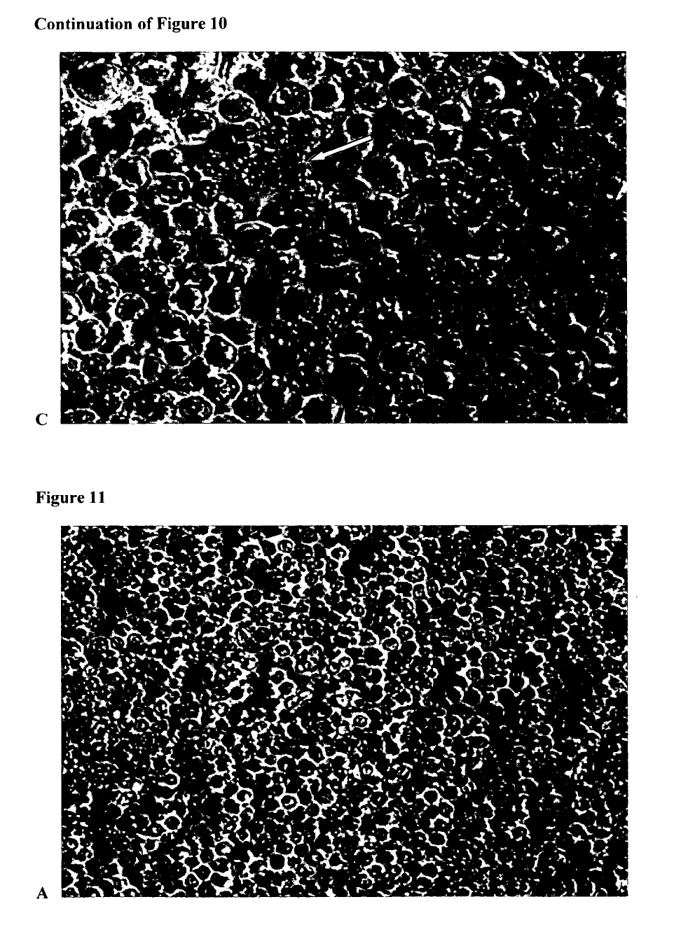

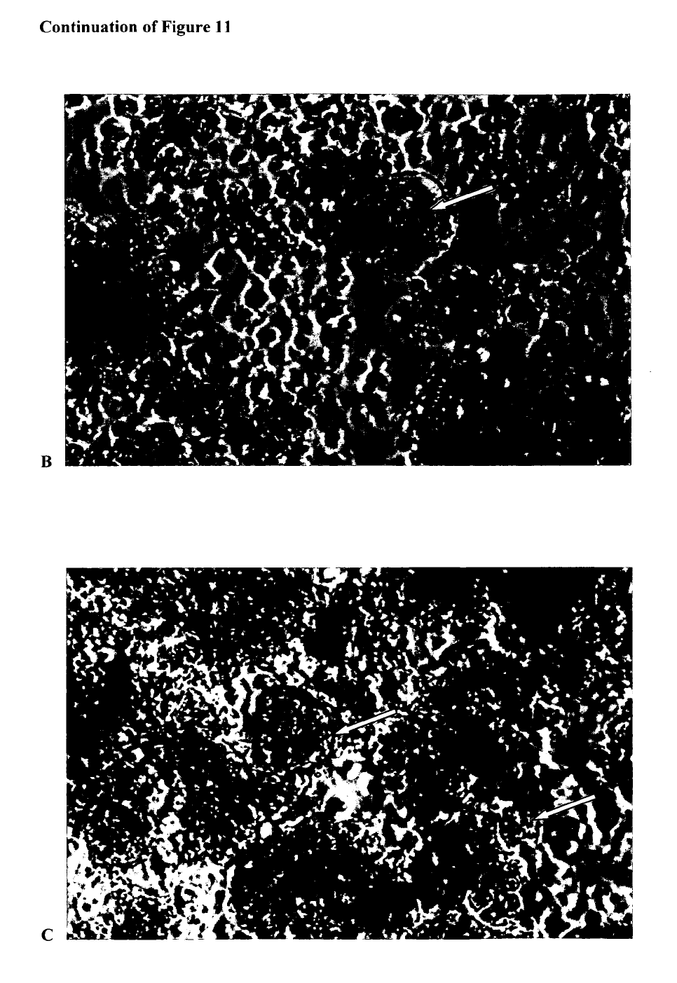

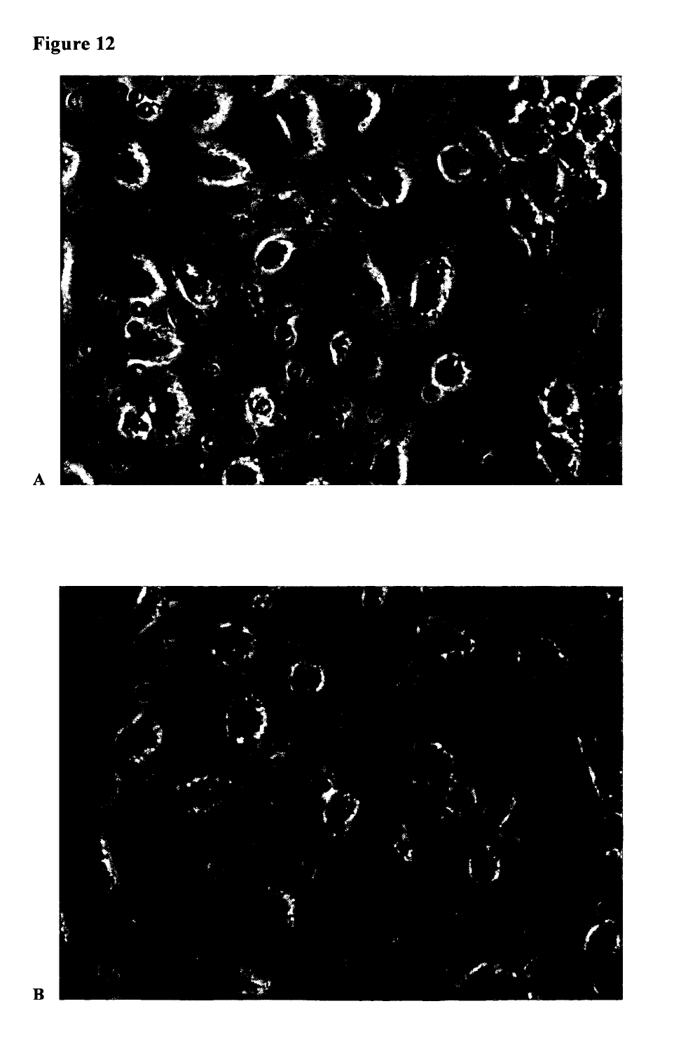

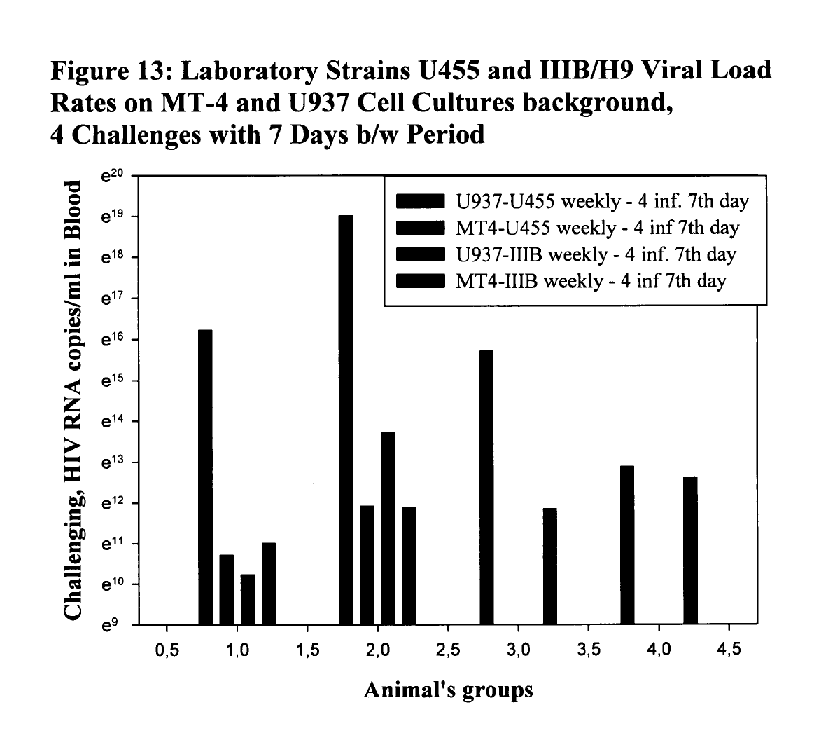

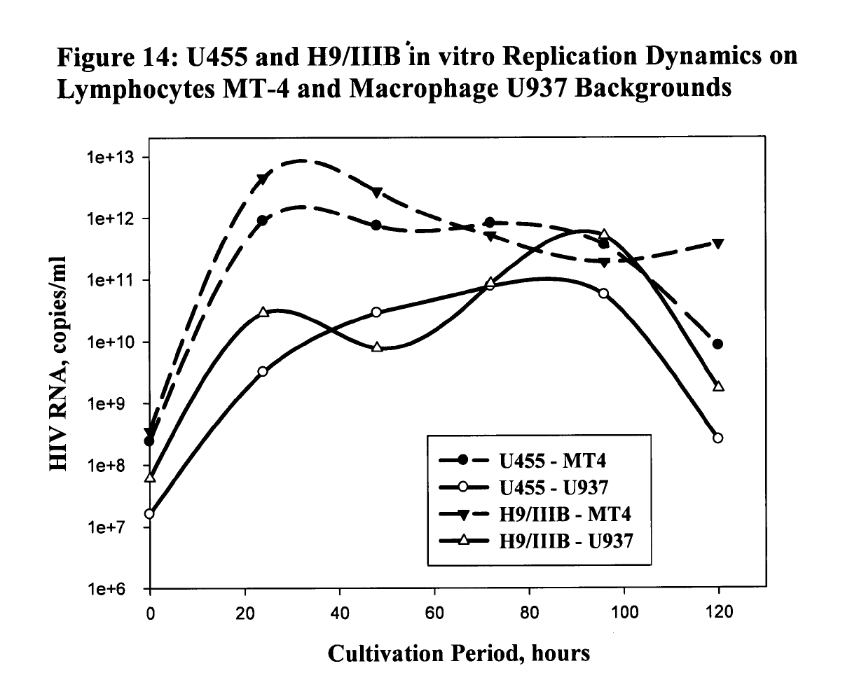

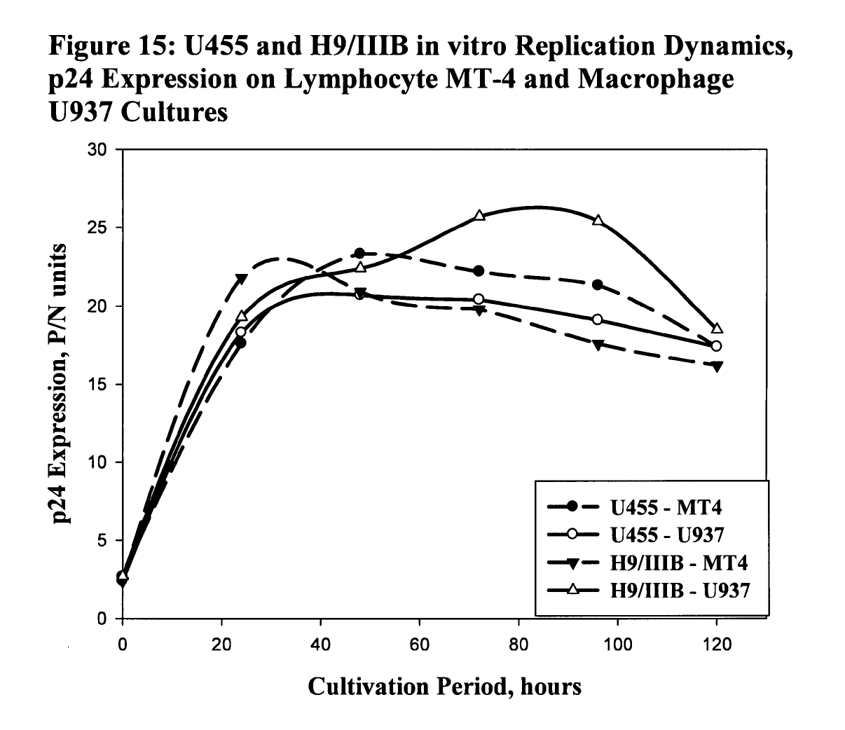

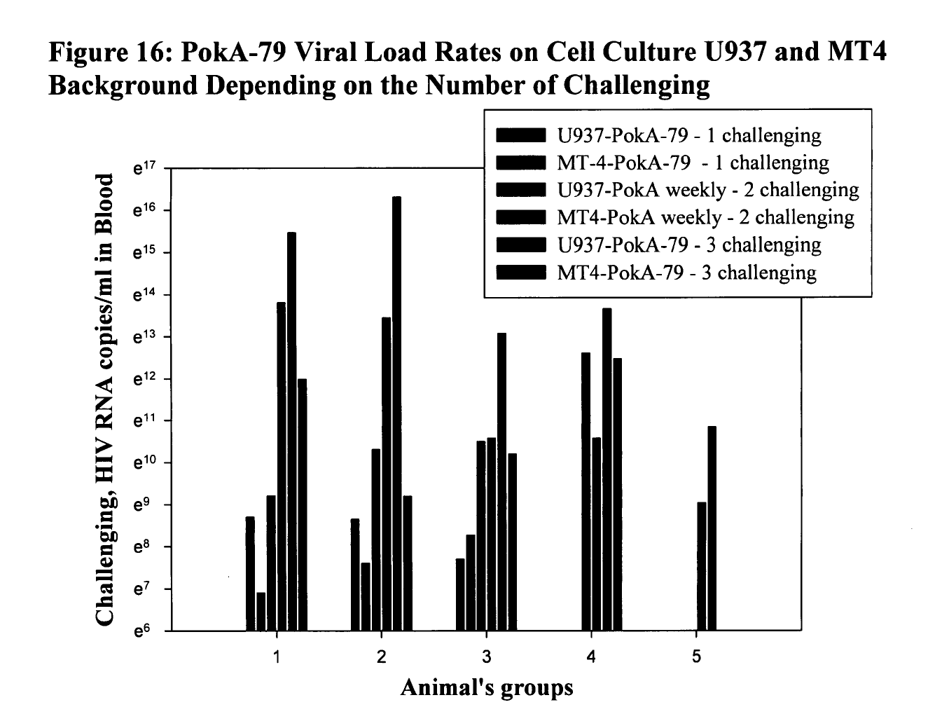

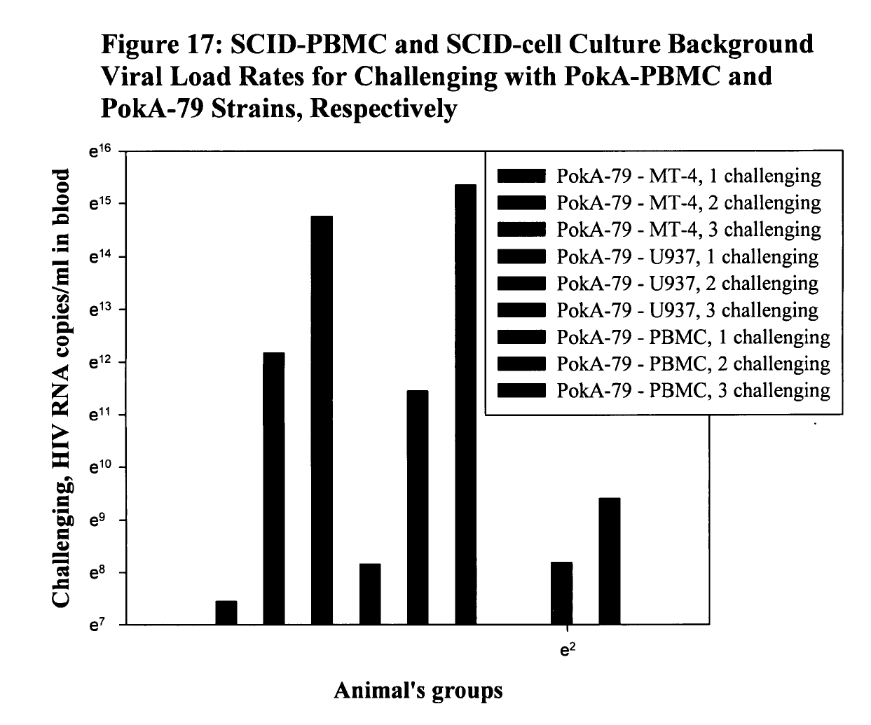

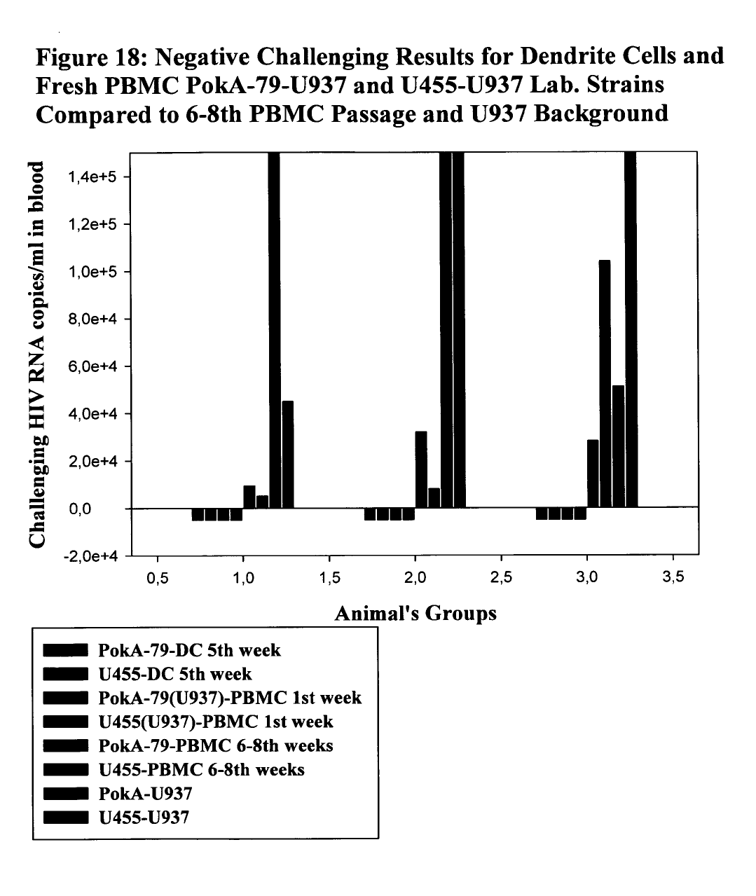

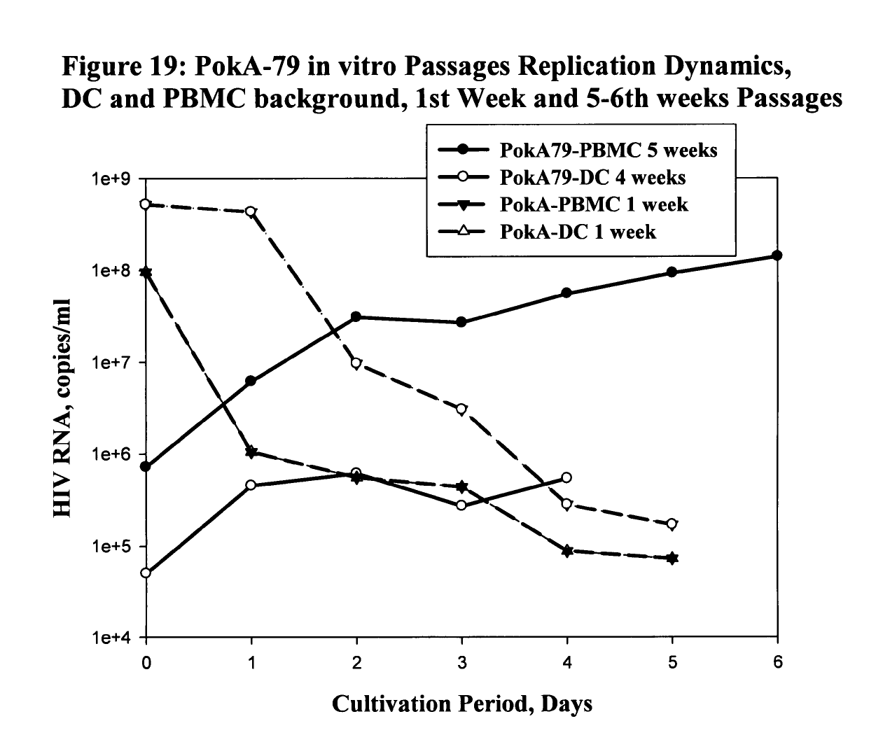

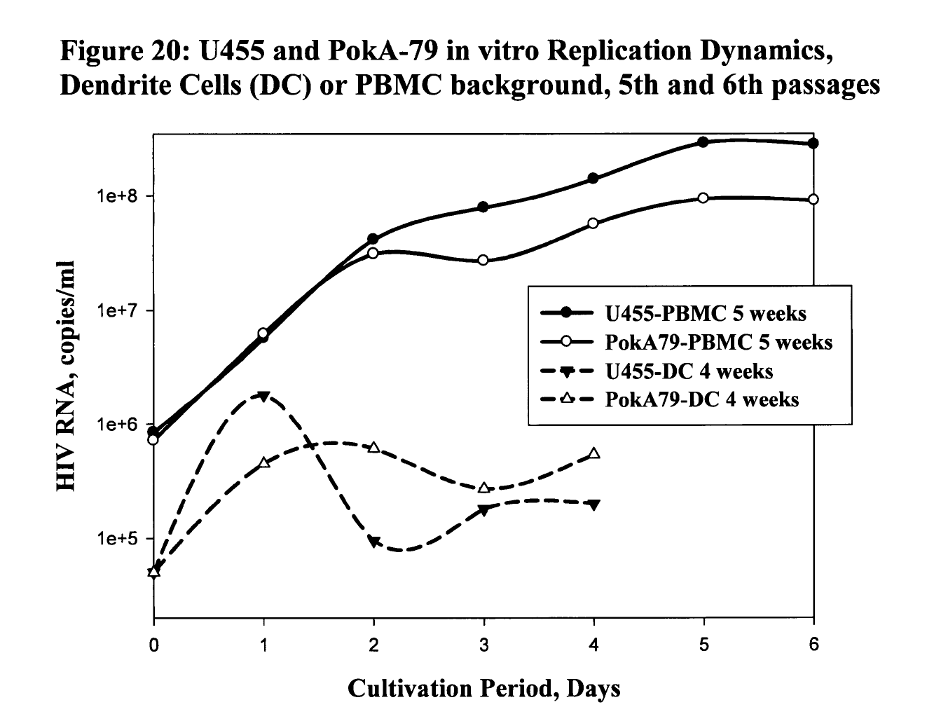

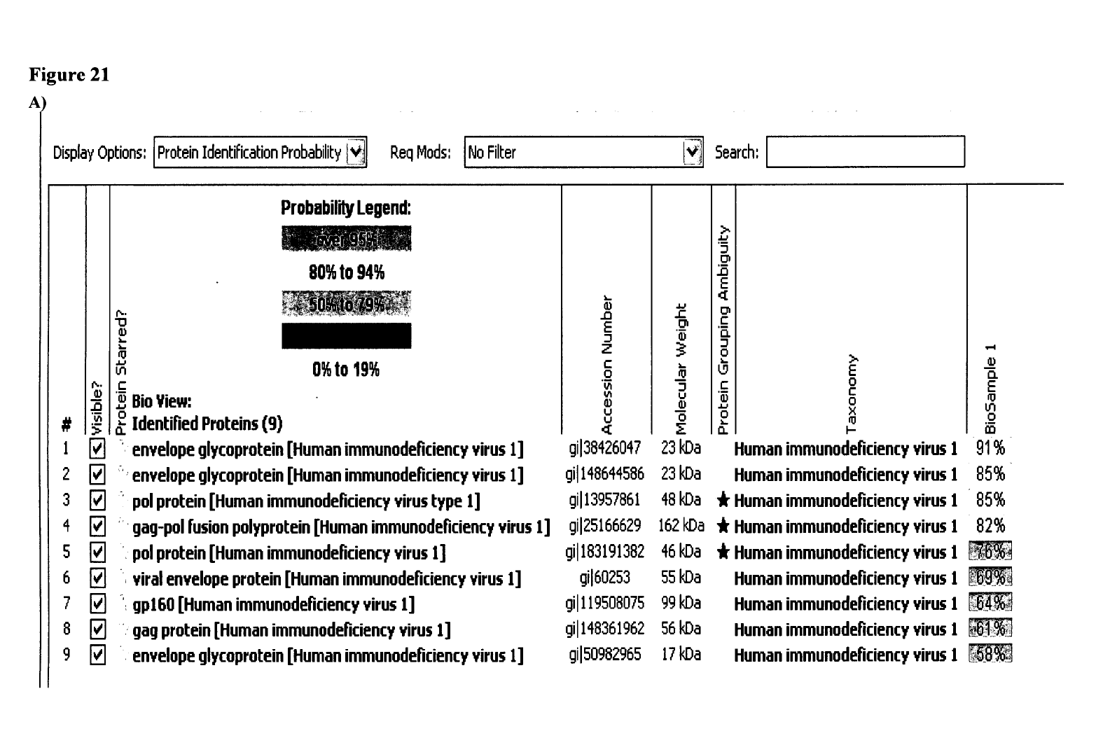

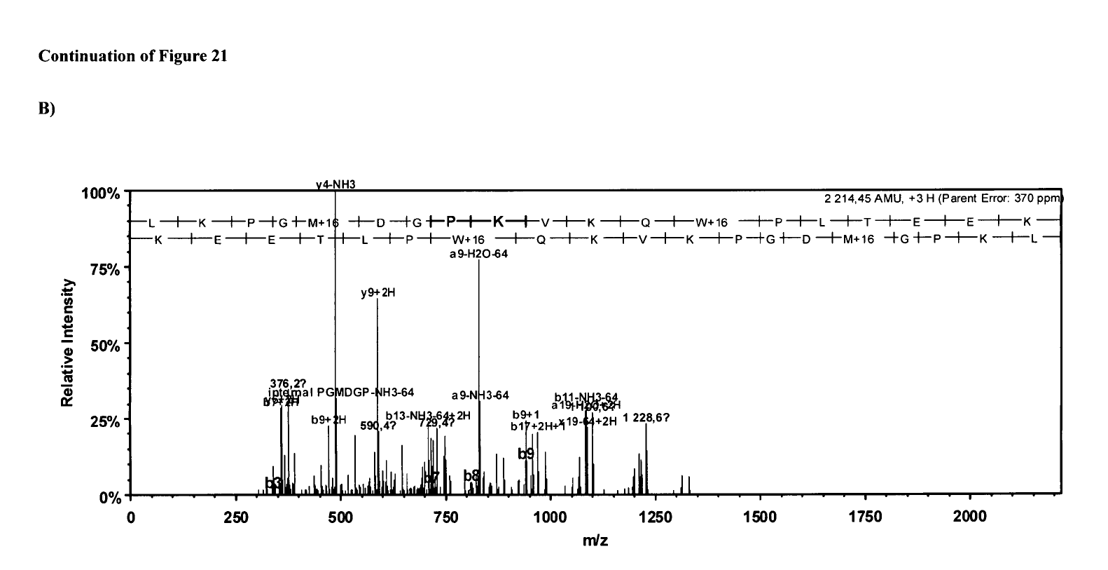

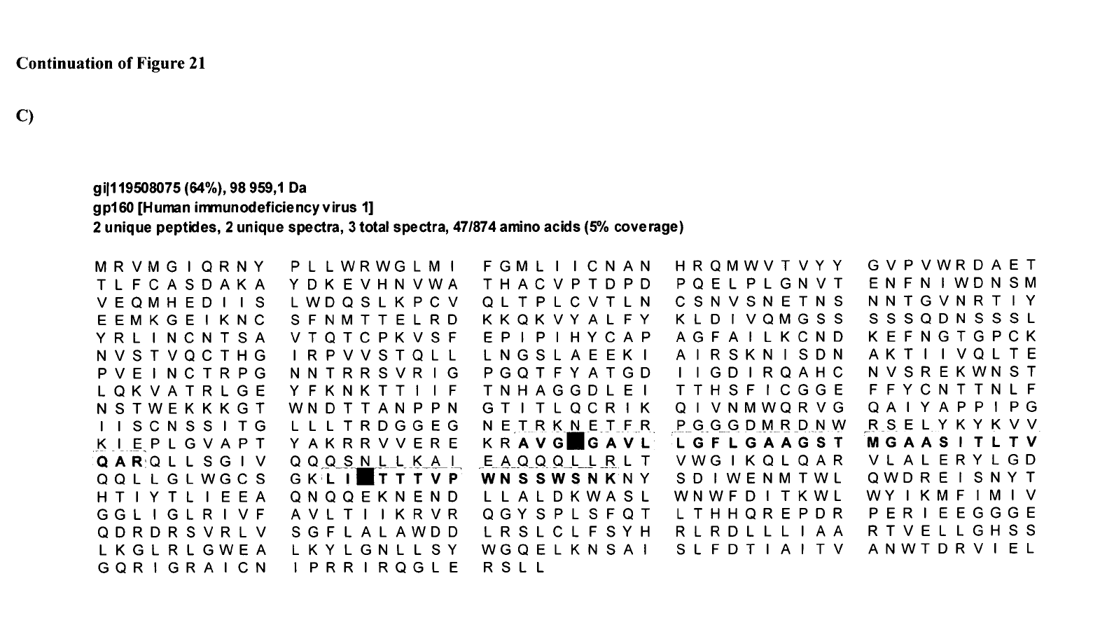

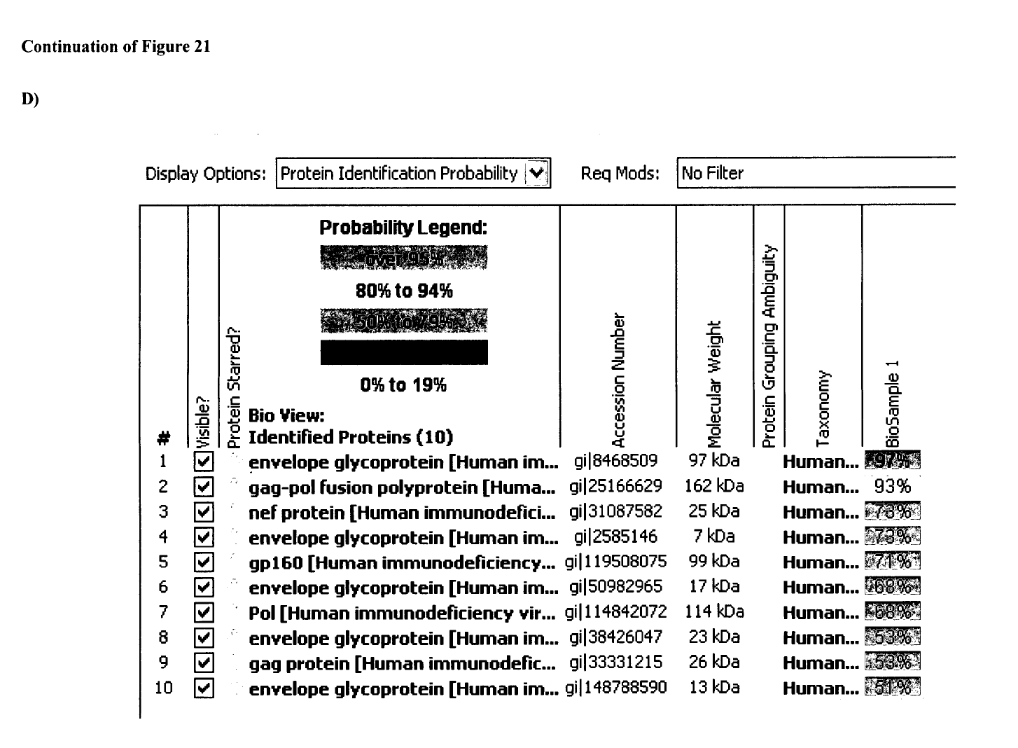

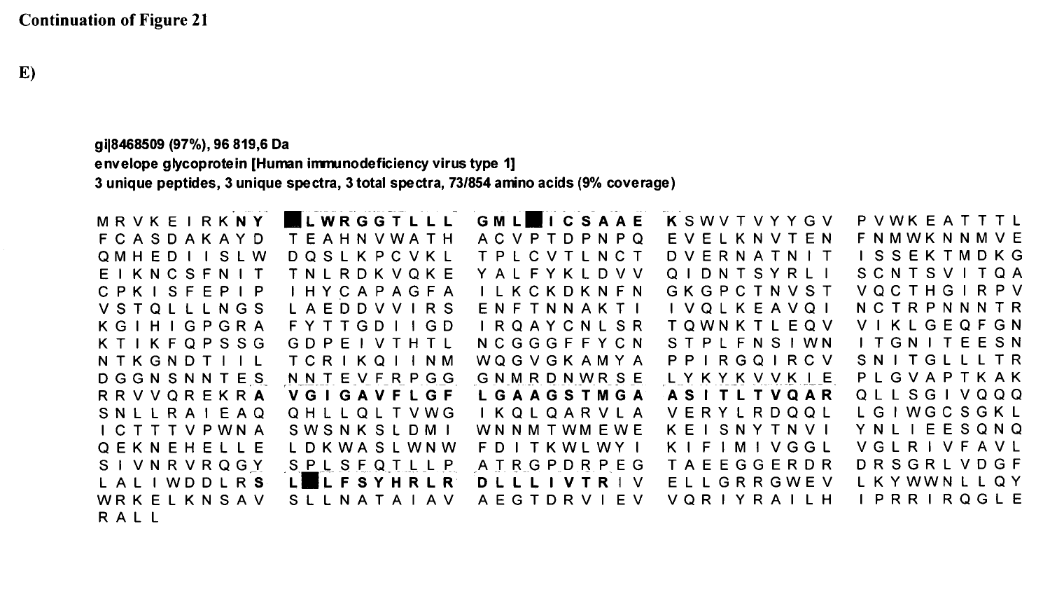

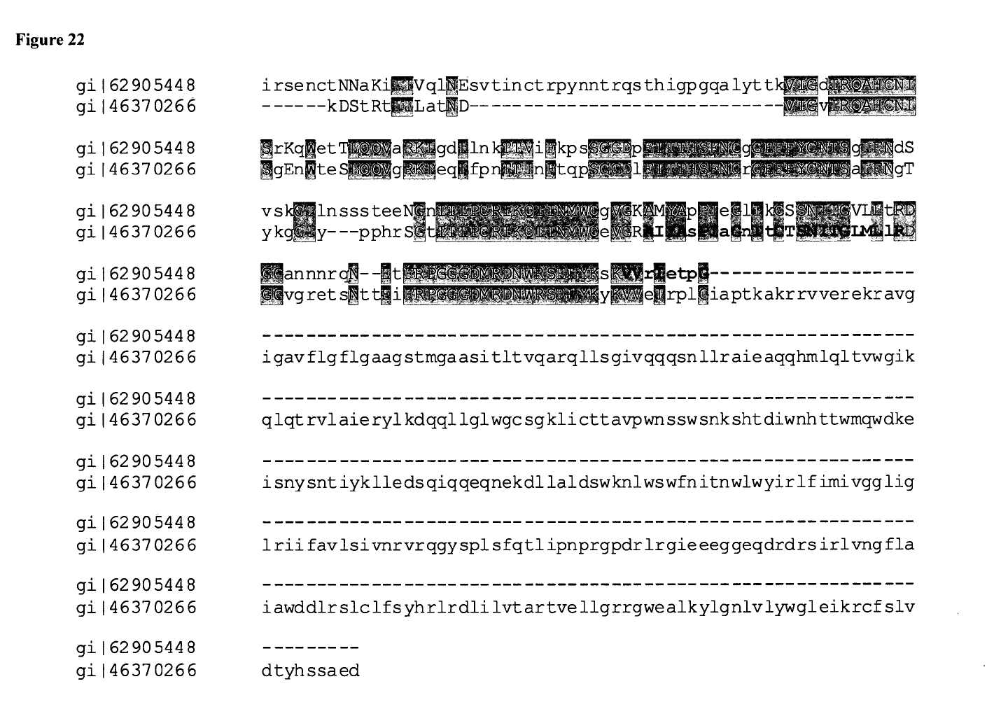

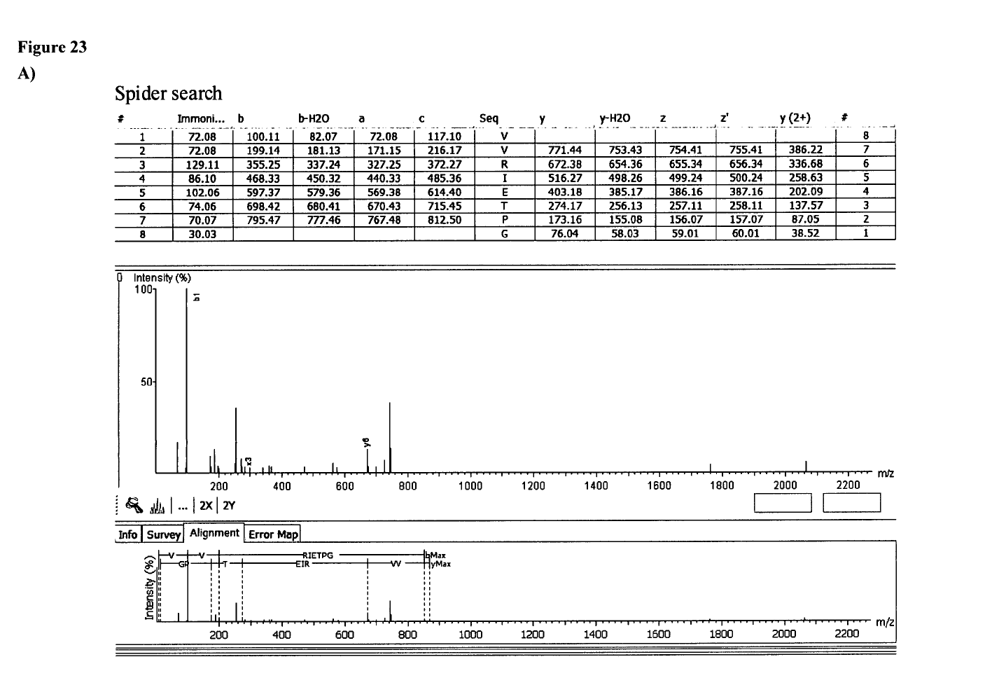

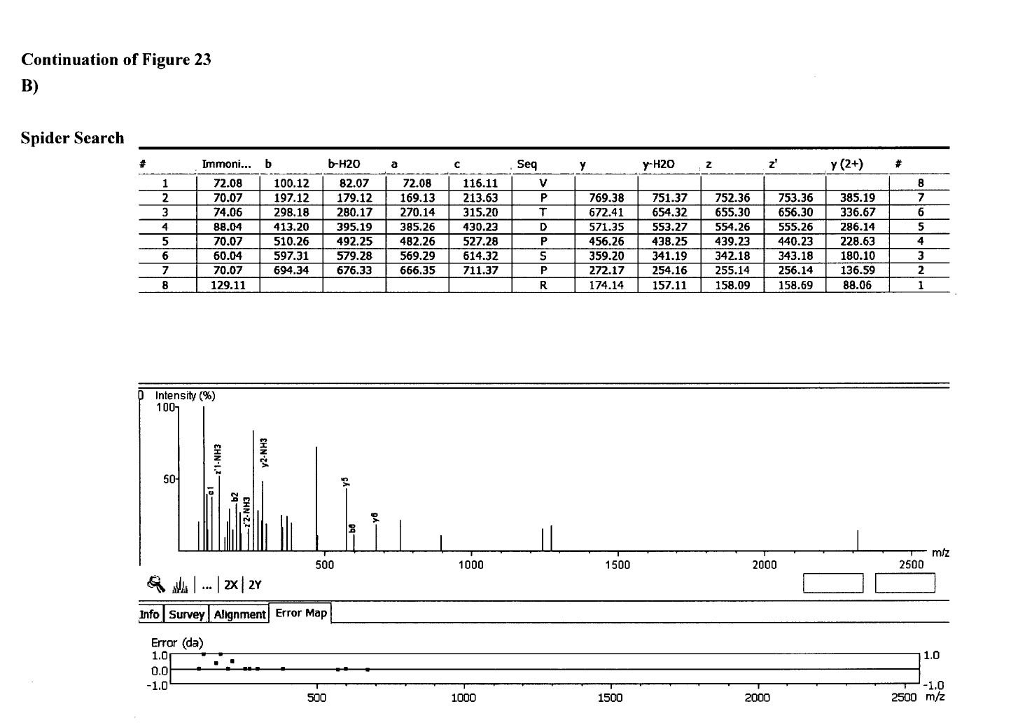

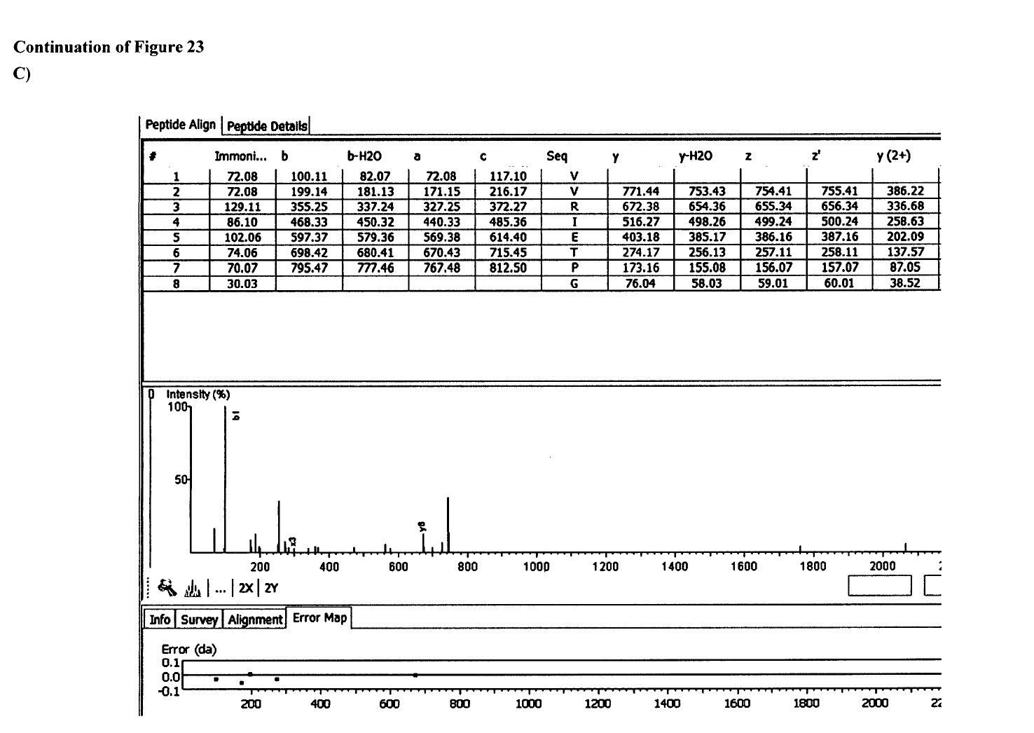

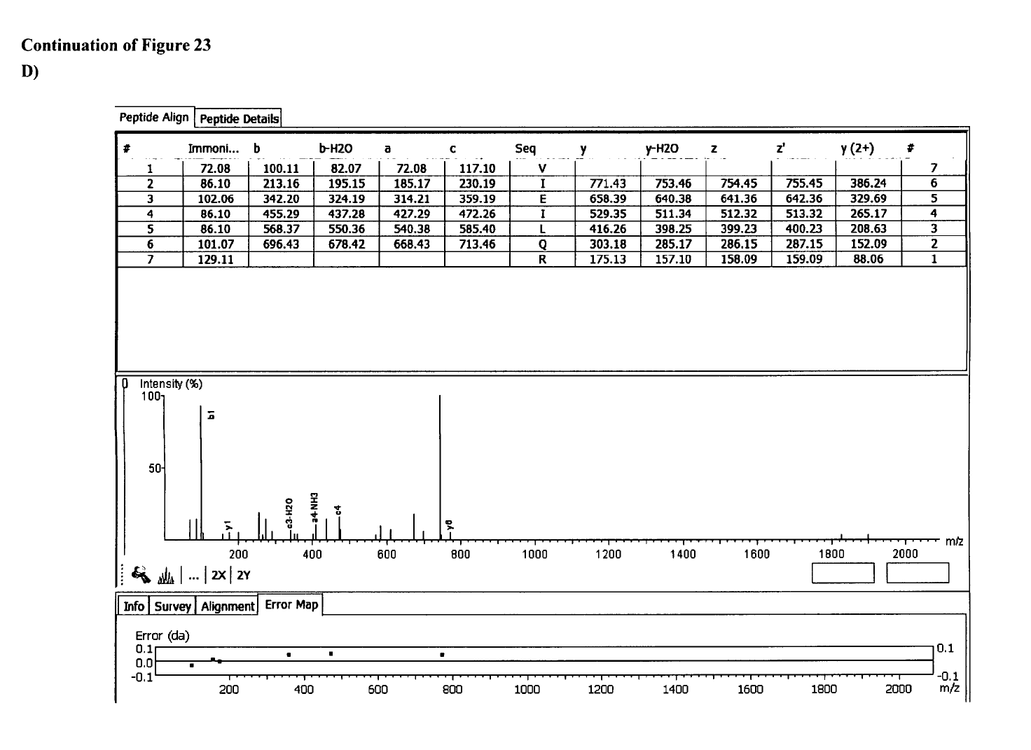

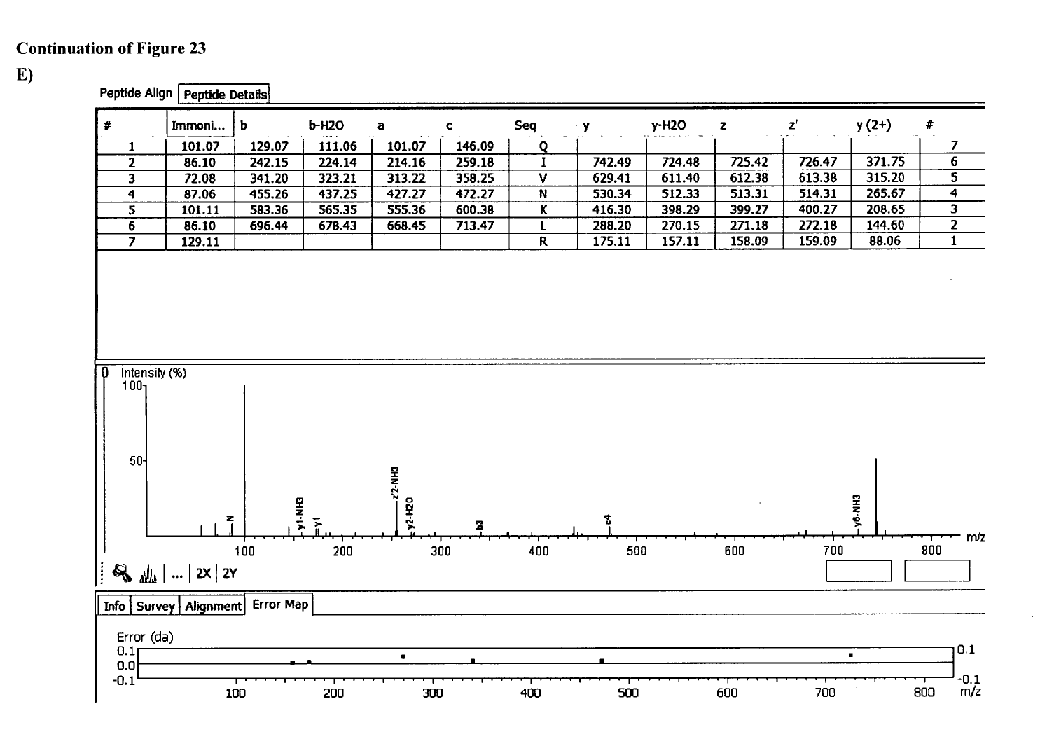

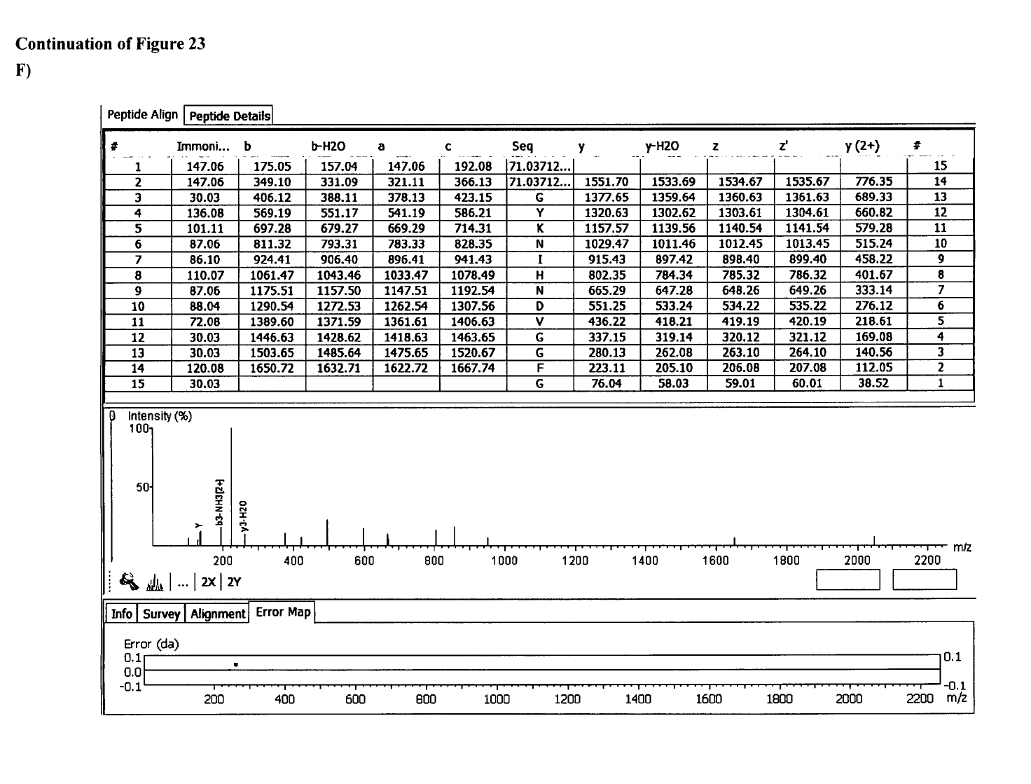

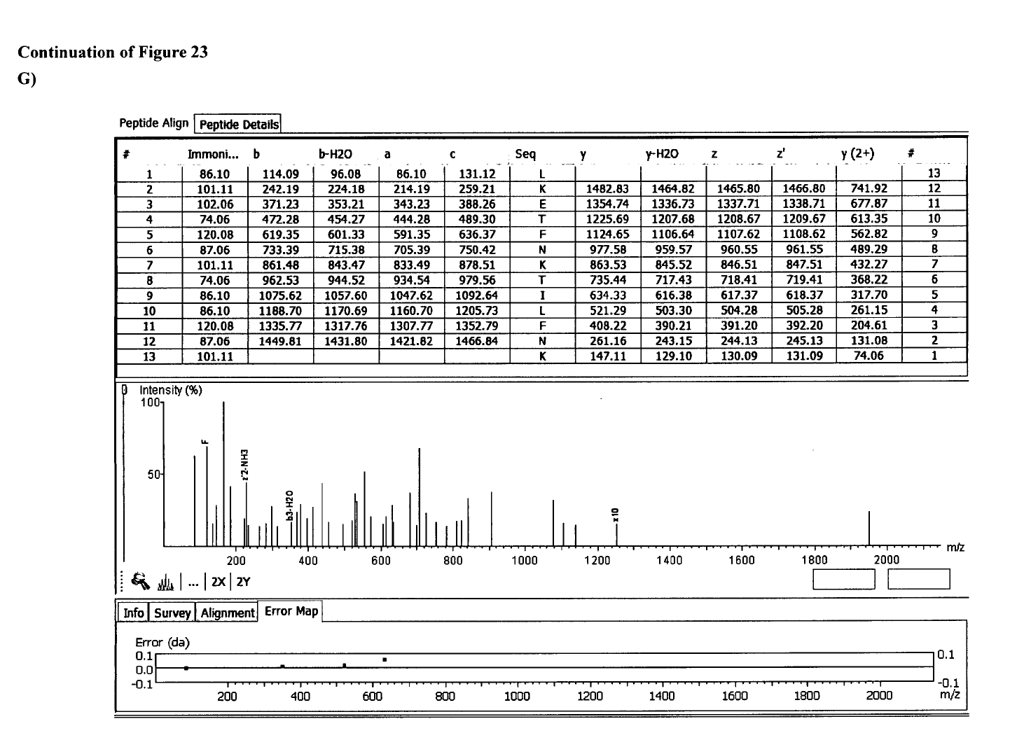

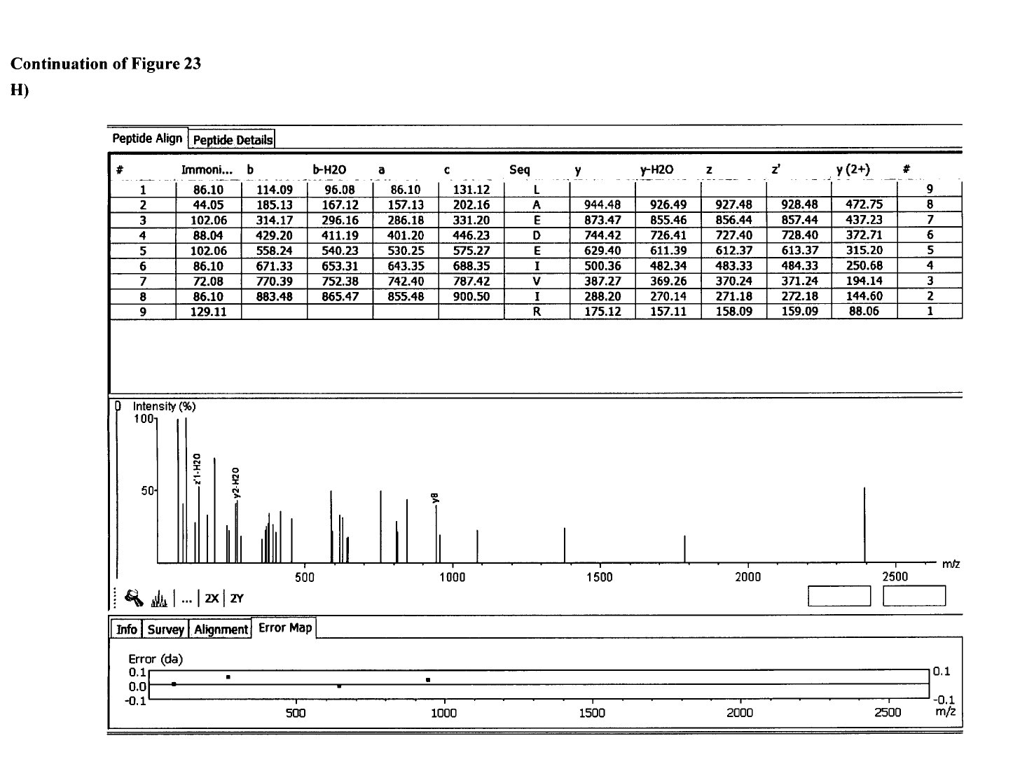

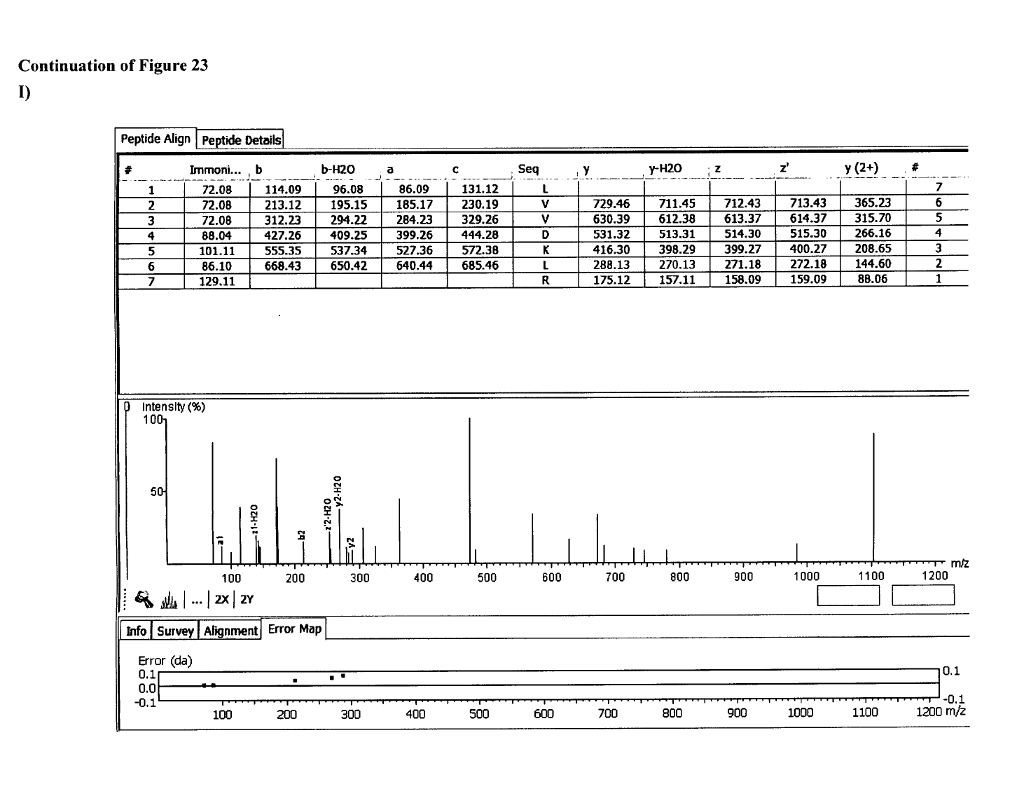

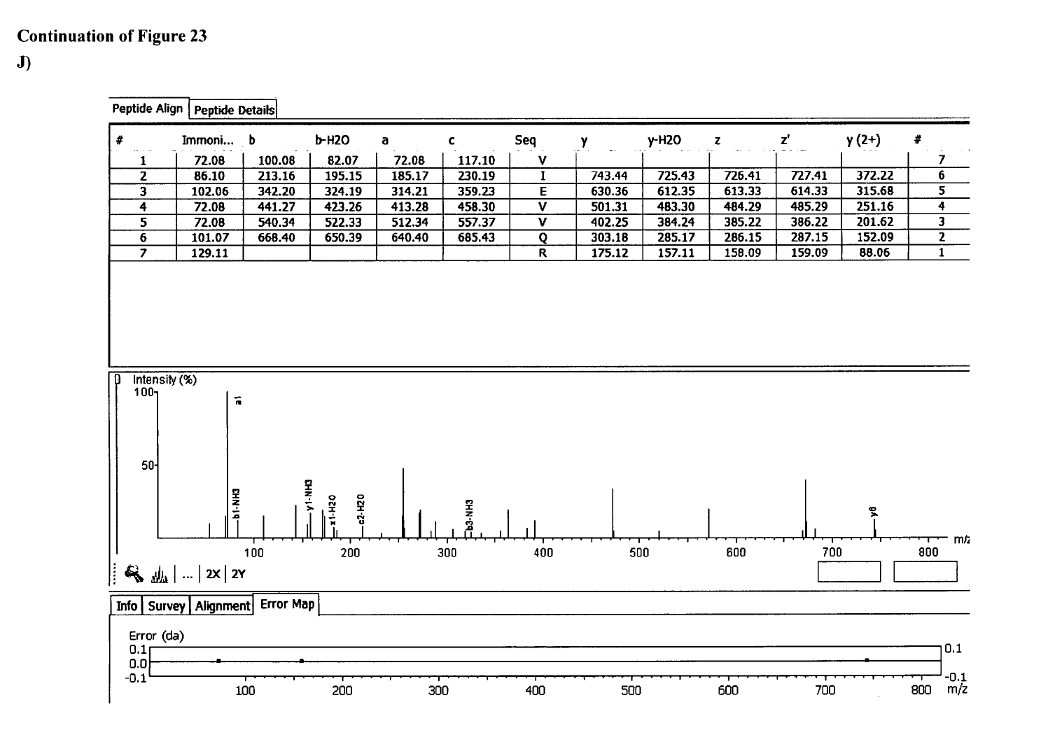

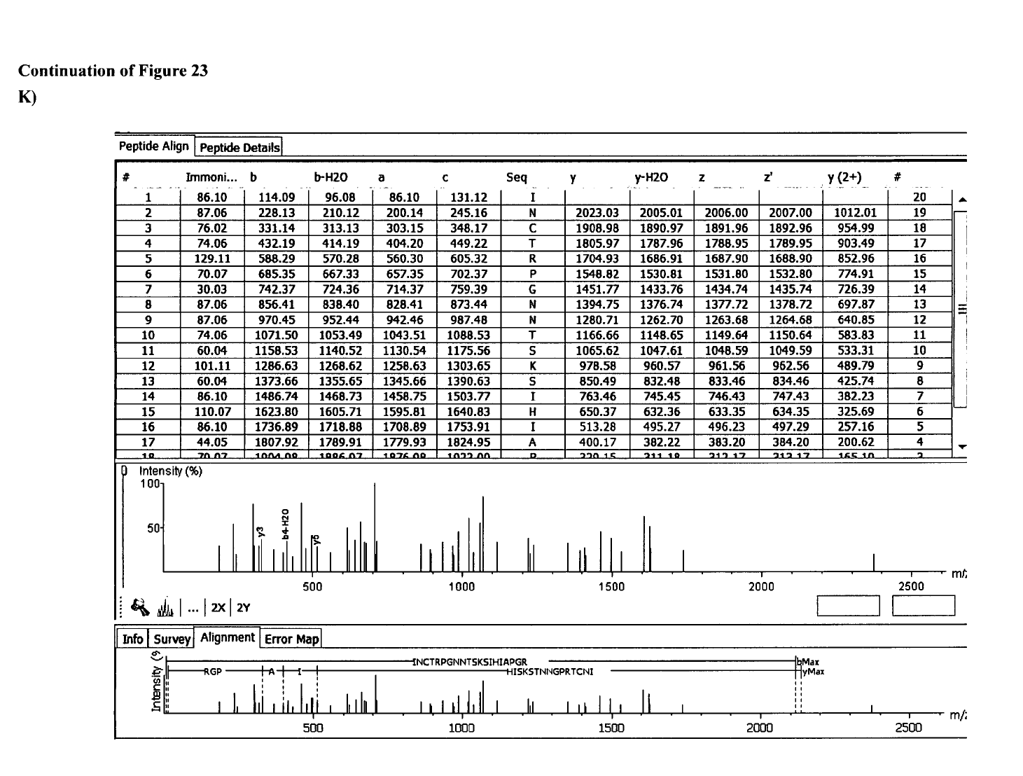

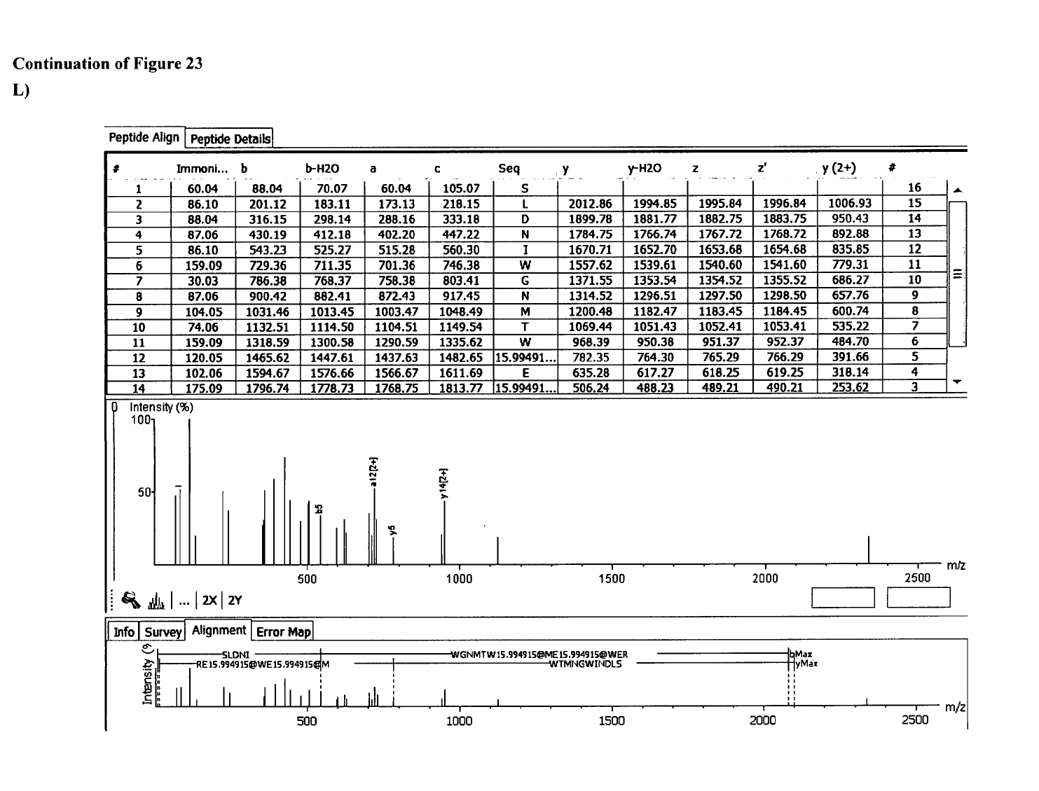

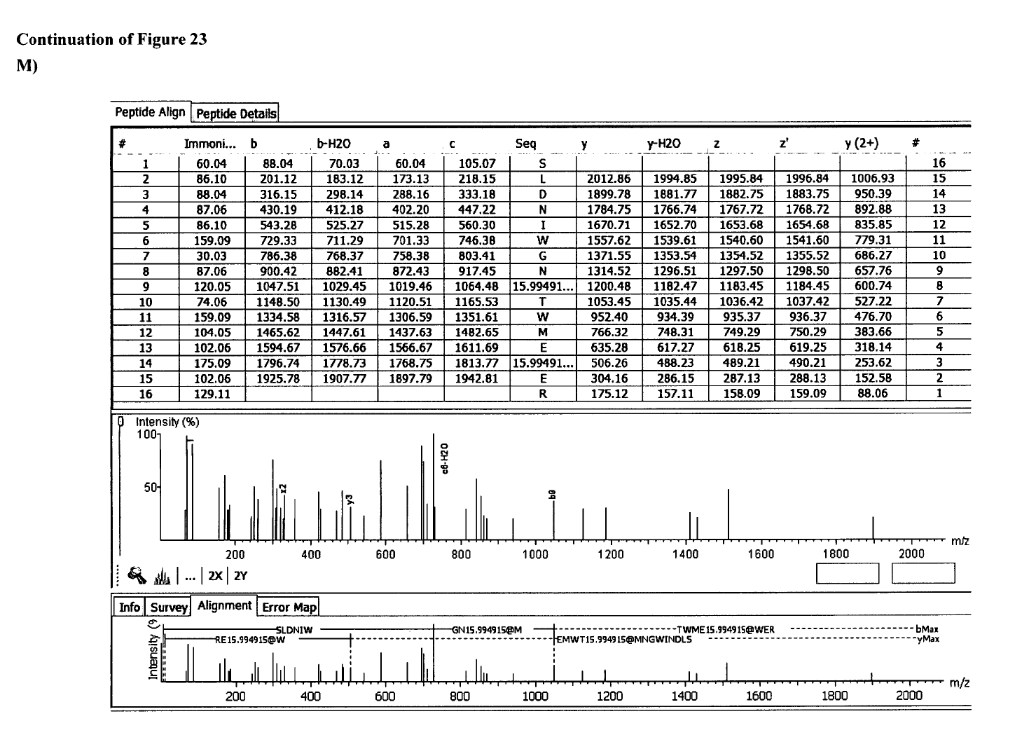

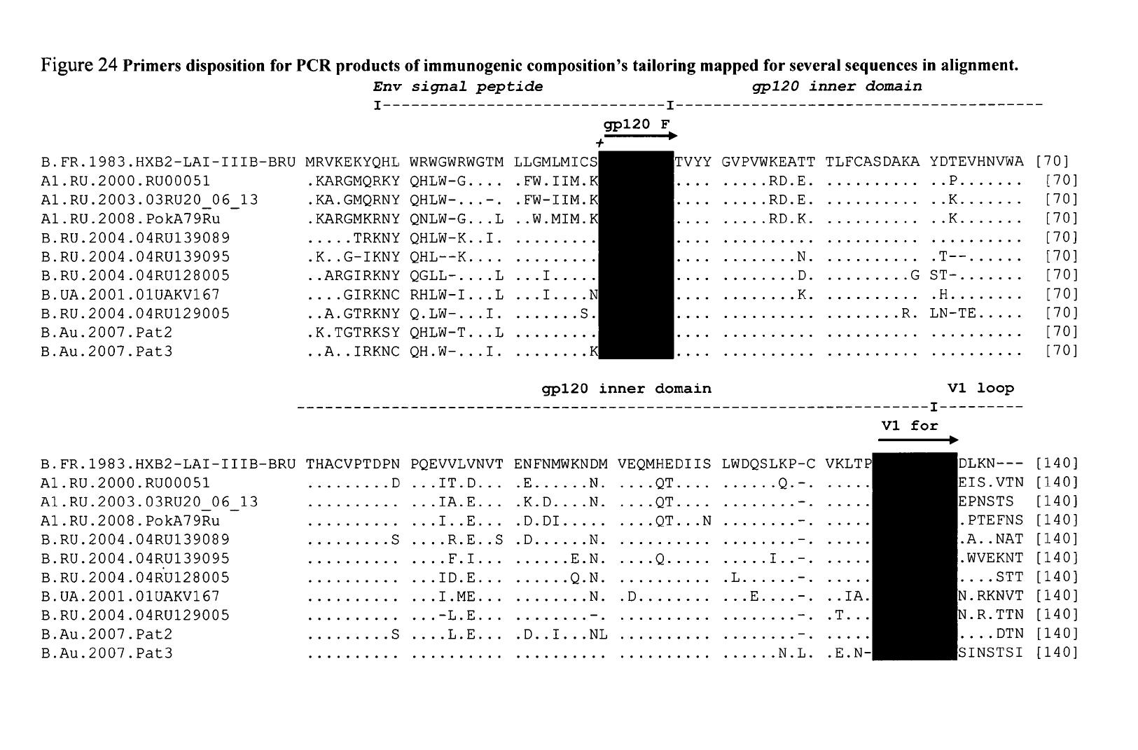

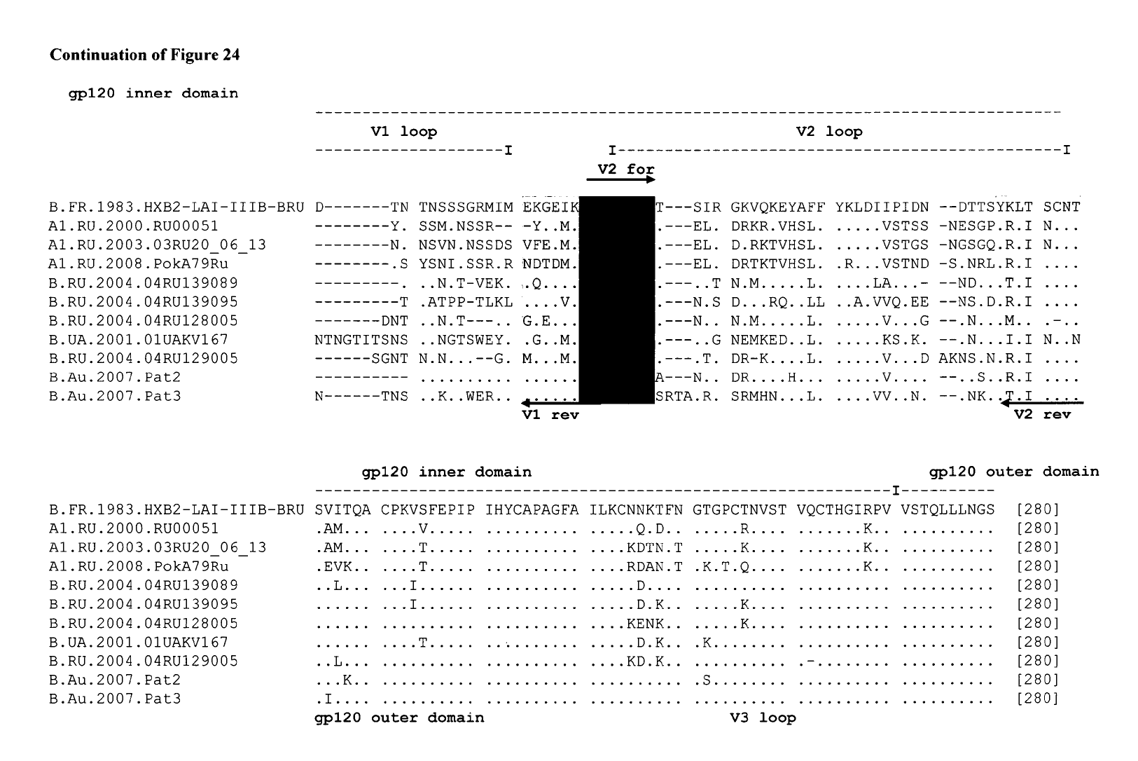

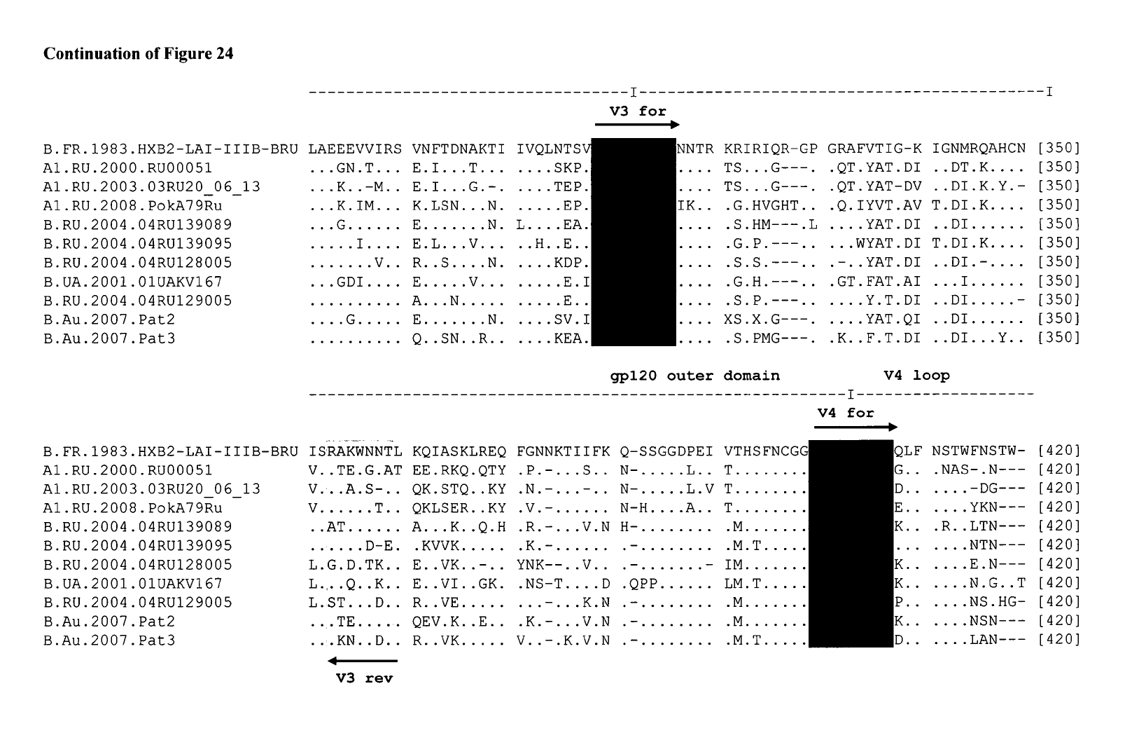

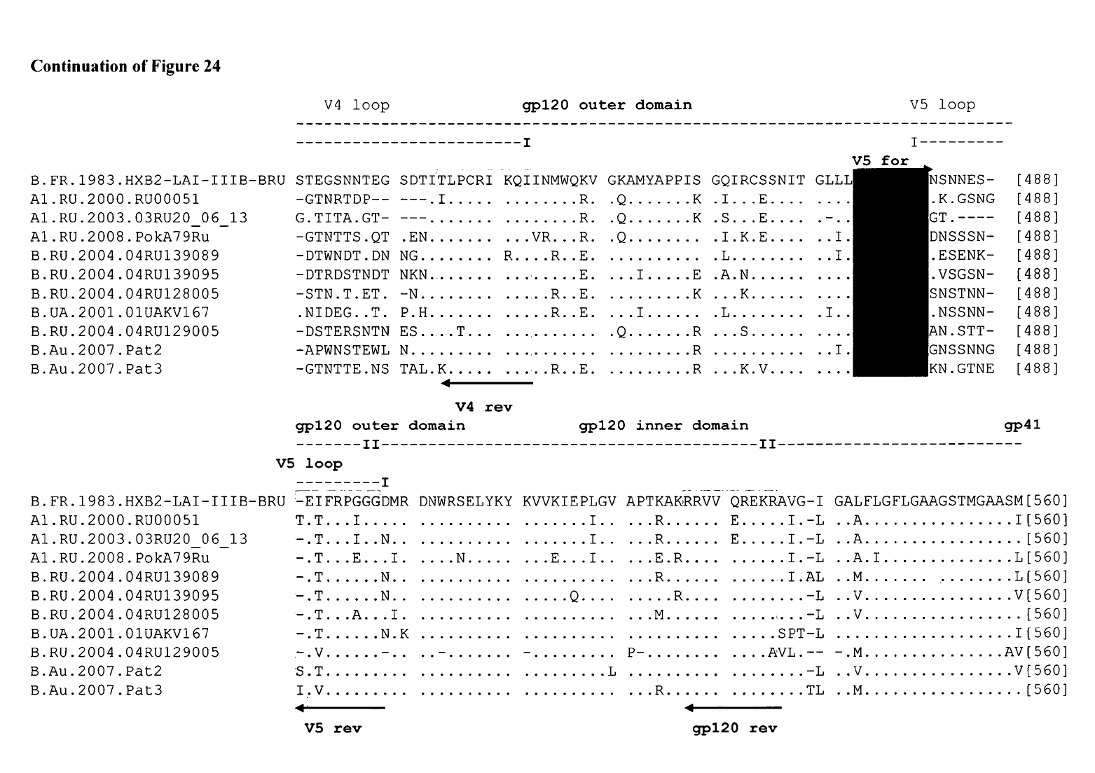

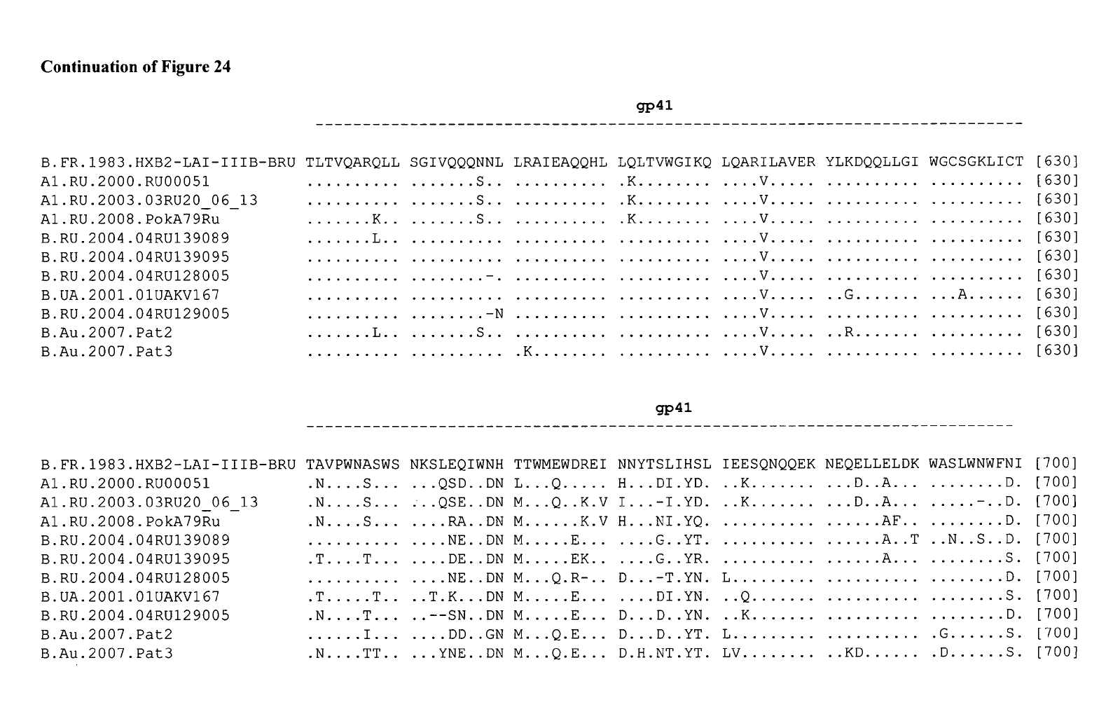

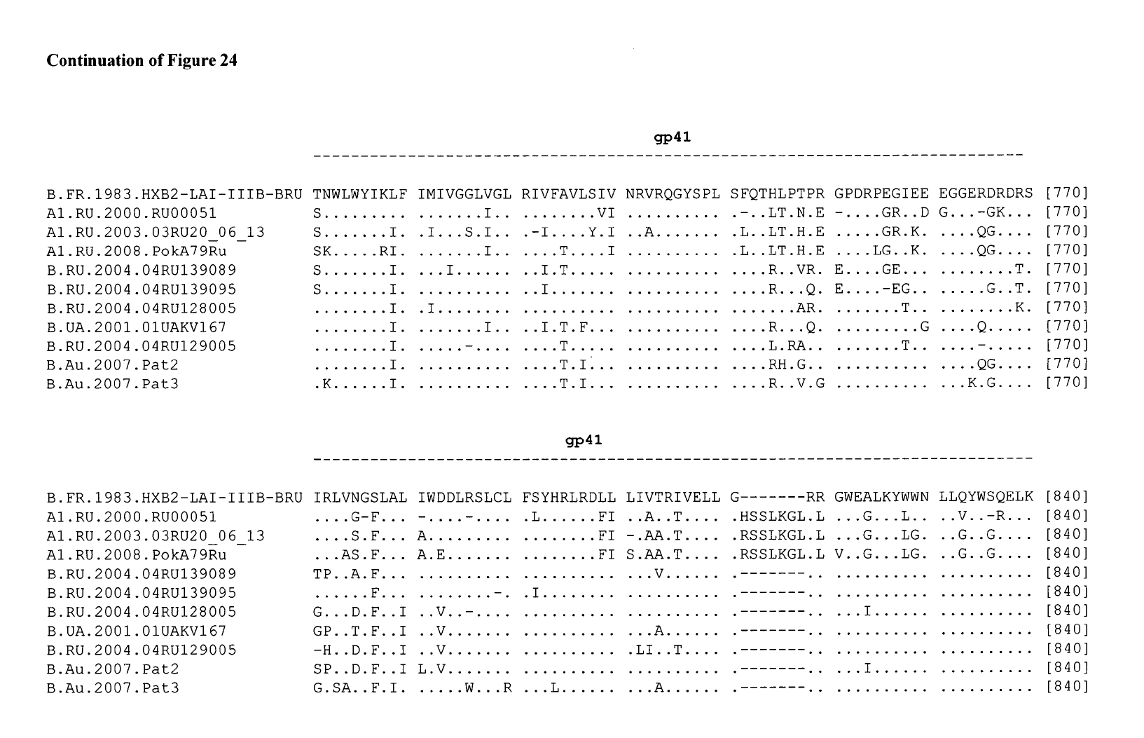

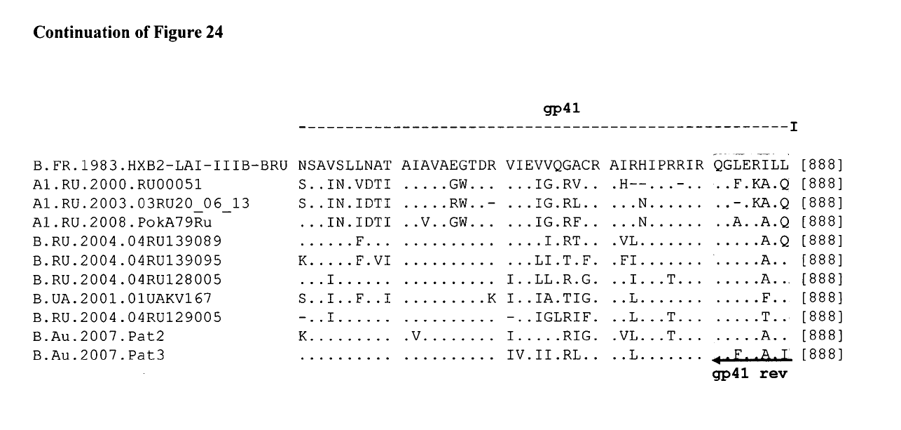

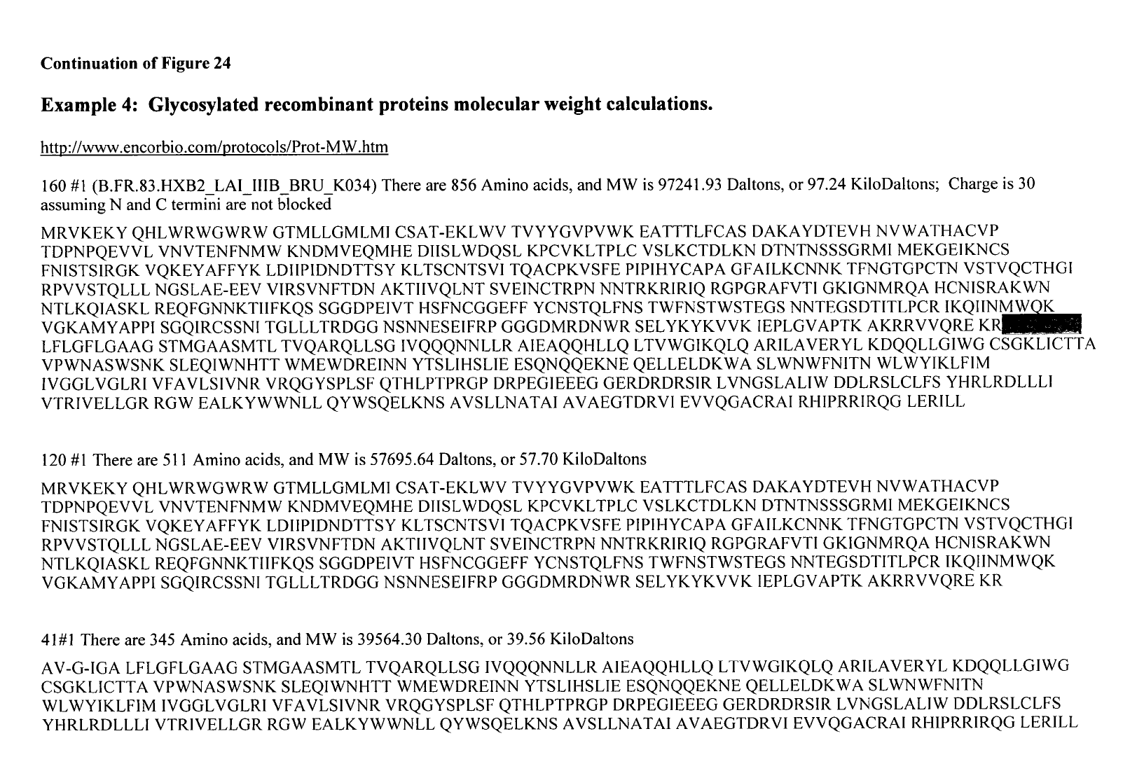

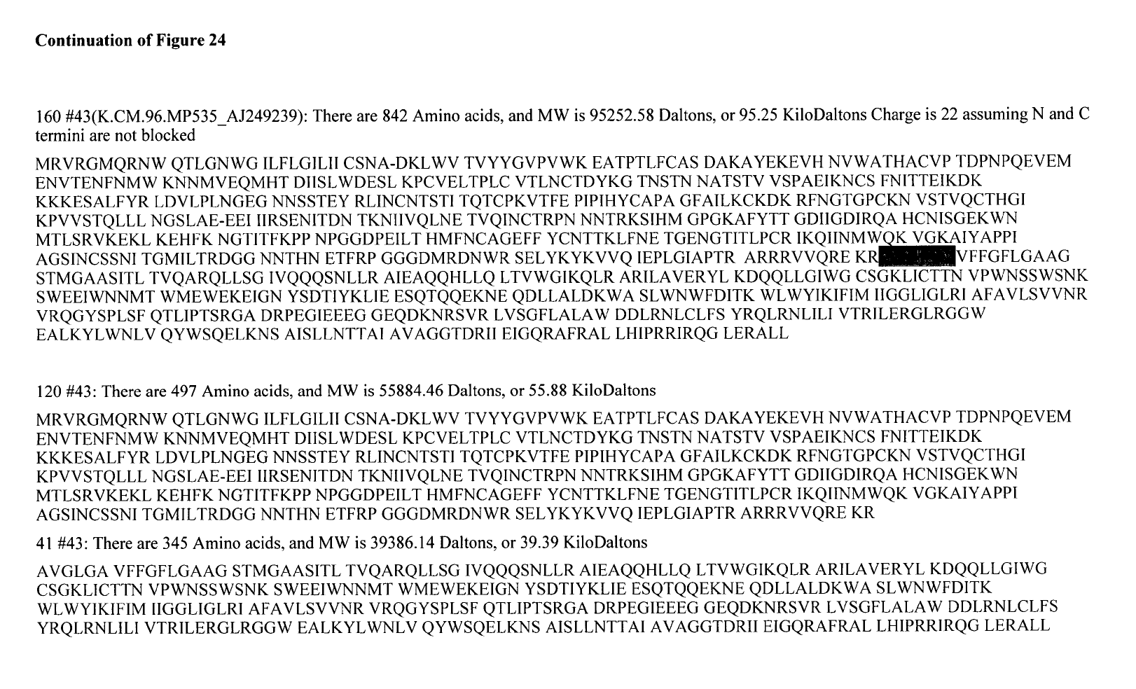

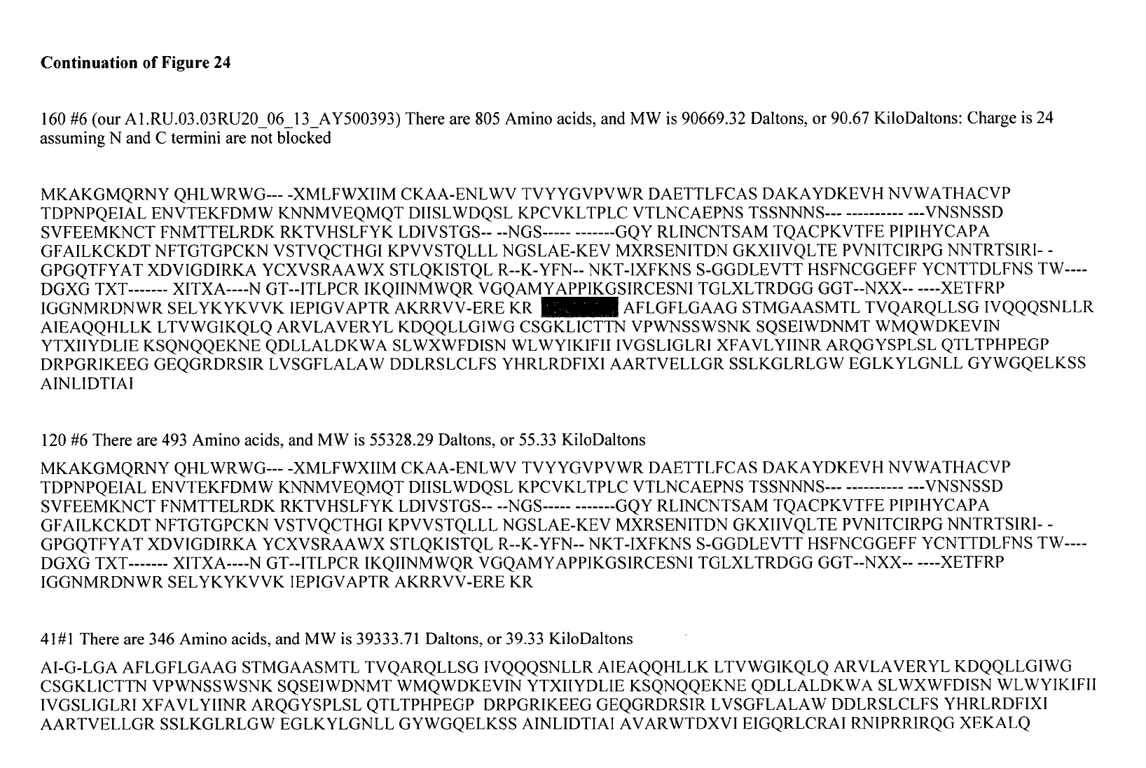

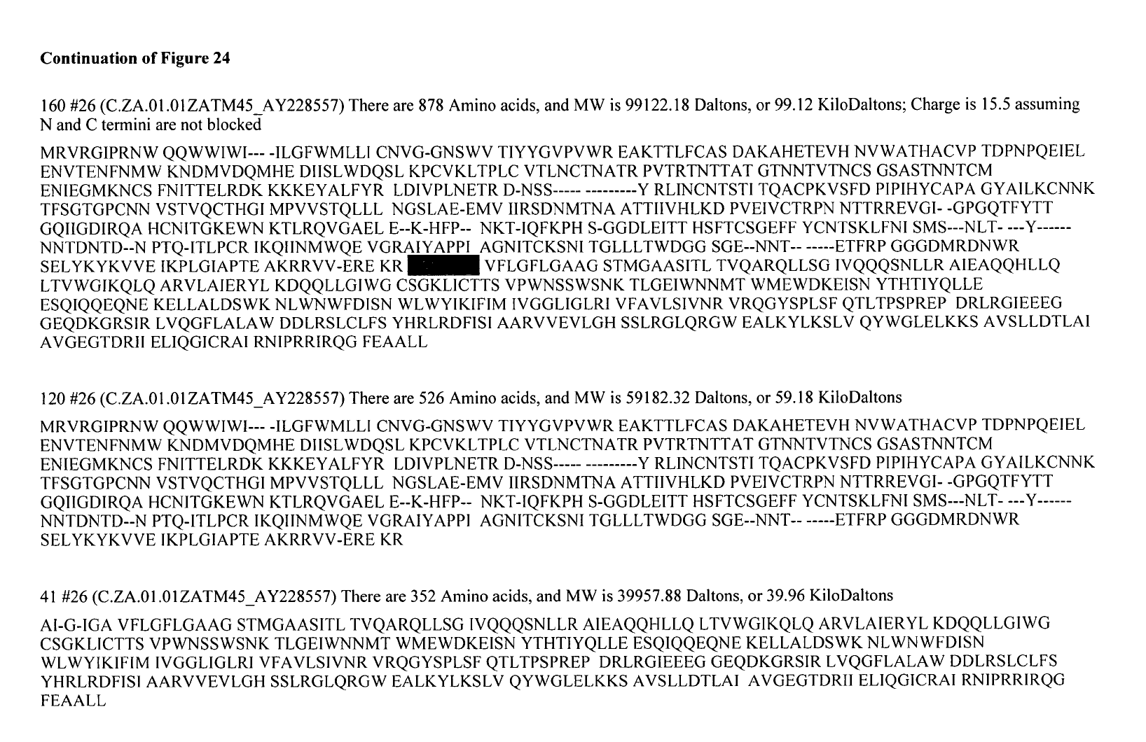

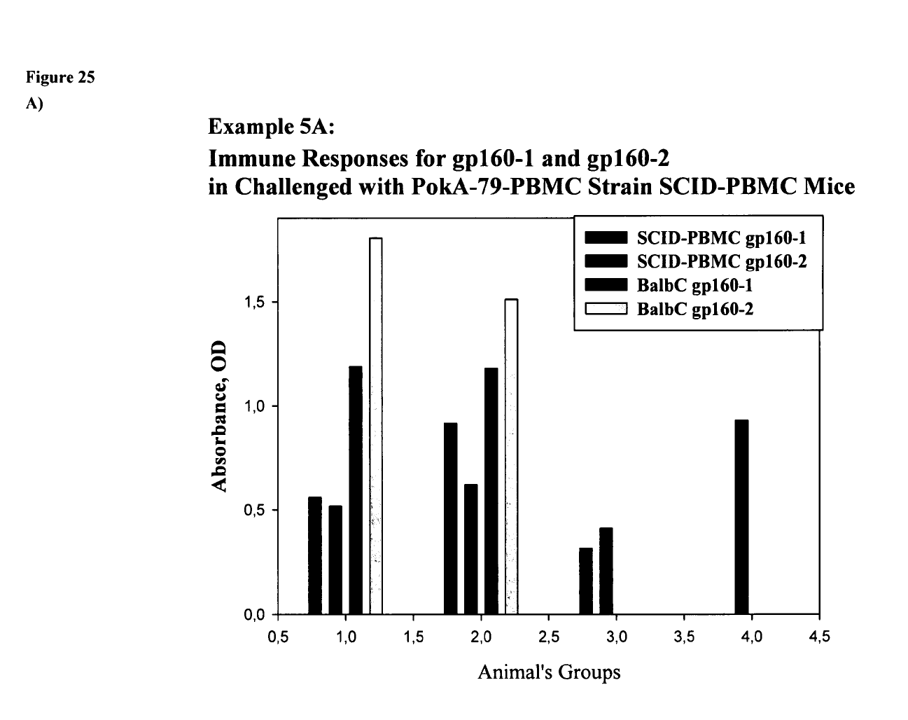

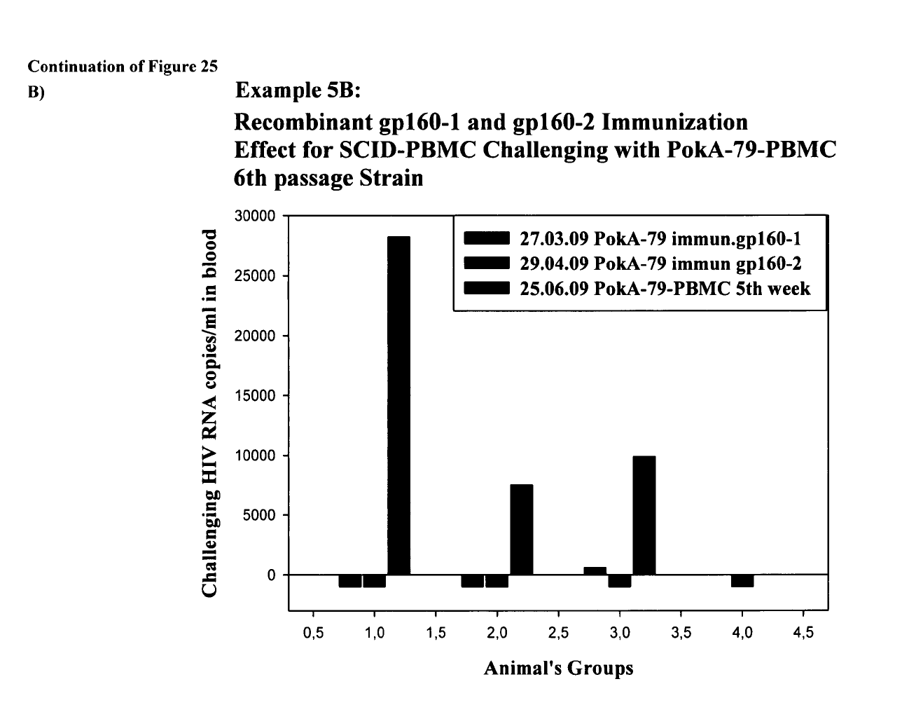

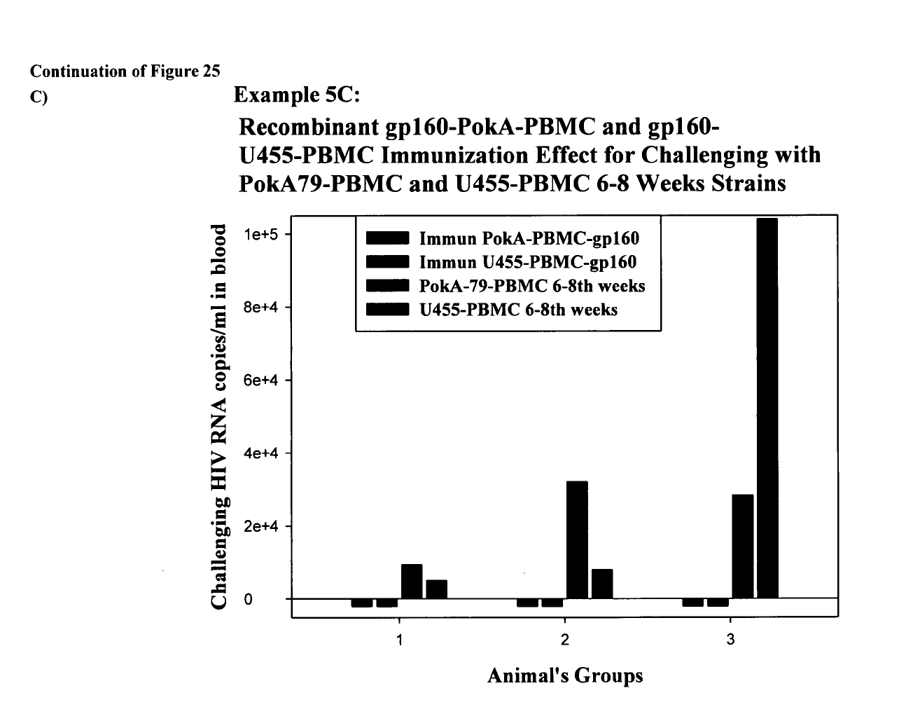

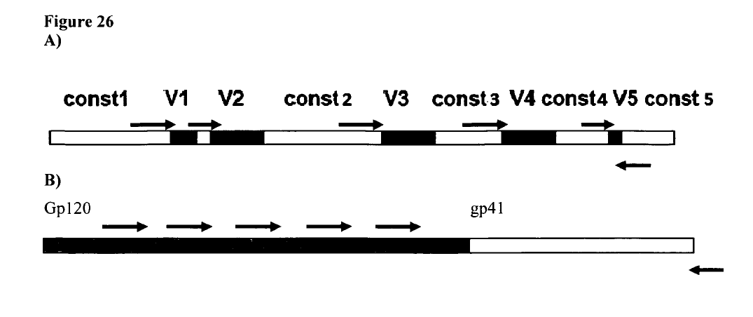

As one can see from microscopy images on FIGS. 9-12 the morphology and functional activity of PBMC (FIG. 11) is settled between monocyte cells presented as U937 culture (FIG. 10) and lymphocytes cells such as MT-4 culture (FIG. 9) but dynamics of their metabolism and multiplication/differentiation is rather matches monocyte cells dynamics. They are more similar to U937 then to dendrite cells (FIG. 12). Therefore the study of in vitro HIV strains titres dynamics and TCID₅₀ parameters for the used for vaccine development's HIV strain or HIV isolate is very important for getting successful results.

According to data received from in vitro dynamics/kinetics study HIV laboratory strains having passage on lymphocytes cell culture background, such as MT-4 or MT-2 in the preferred embodiment, have log phase in virus RNA copies titre on the 2^{nd} day of fresh cells infection cultivation and should be challenged to Hu-SCID animals 24-36 hours after the passage (Table 2). HIV laboratory strains having passage on monocytes cell culture background, such as U937 in the preferred embodiment, have log phase in virus RNA copies titre on the 3^{rd}-5th day of fresh cells infection cultivation and should be challenged to Hu-SCID animals 48-60 hours after the passage (Table 2). Positive challenging data are received for these regimes of infectious material intraperitoneal administration (FIGS. 4-6, 13, 16). The in vitro passages dynamics for PBMC-HIV “playback” strains is similar to monocyte-type cell cultures (Table 5). Thus, Positive challenging results can be obtained for 3^{rd} day of passage cultivation PBMC-PokA79, PBMC-U455, etc. infectious material intraperitoneal administrations (FIGS. 17-18).

TABLE 2
Titre and dynamics of HIV laboratory strains viral replication on lymphocyte-
type and monocyte-type cultivation cell cultures, p24 and RNA copies/ml

| | | Time of Incubation Period, hours | | | | | | | | | | | |
|---|---|---|---|---|---|---|---|---|---|---|---|---|---|
| | | 0 | | 24 | | 48 | | 72 | | 96 | | 120 | |
| HIV Lab. | Cell | p24, | RNA, | p24, | RNA, | p24, | RNA, | p24, | RNA, | p24, | RNA, | p24, | RNA, |
| Strain | Culture | P/N | c/ml | P/N | c/ml | P/N | c/ml | P/N | c/ml | P/N | c/ml | P/N | c/ml |
| U455 | MT-2 | 2.3 | 4.8 × 10⁸ | 18.1 | 3.3 × 10¹¹ | 22.5 | 5.2 × 10¹¹ | 14.4 | 3.4 × 10¹¹ | 14.1 | 9.5 × 10¹⁰ | 8.8 | 9.2 × 10⁸ |
| | MT-4 | 2.7 | 2.4 × 10⁸ | 17.6 | 8.9 × 10¹¹ | 23.3 | 7.4 × 10¹¹ | 22.2 | 8.1 × 10¹¹ | 21.3 | 3.6 × 10¹¹ | 17.4 | 8.4 × 10⁹ |
| | U937 | 2.4 | 1.6 × 10⁷ | 18.3 | 3.9 × 10⁹ | 20.7 | 2.9 × 10¹⁰ | 20.4 | 7.7 × 10¹⁰ | 19.1 | 5.6 × 10¹⁰ | 17.4 | 2.5 × 10⁸ |
| H9/IIIB | MT-2 | 3.0 | 5.9 × 10⁷ | 20.7 | 5.7 × 10¹² | 22.1 | 3.8 × 10¹² | 22.3 | NM* | 22.4 | NM* | 13.0 | NM* |
| | MT-4 | 2.4 | 3.5 × 10⁸ | 21.8 | 4.5 × 10¹² | 20.9 | 2.7 × 10¹² | NM* | NM* | NM* | NM* | NM* | NM* |
| | U937 | 2.7 | 6.1 × 10⁷ | 19.3 | 2.9 × 10¹⁰ | 22.4 | 7.8 × 10⁹ | 25.7 | 8.5 × 10¹⁰ | 25.4 | 5.1 × 10¹¹ | 18.5 | 1.7 × 10⁹ |
| PokA-79 | MT-2 | 2.0 | 4.2 × 10⁸ | 16.2 | 8.2 × 10¹² | 20.1 | 2.3 × 10¹³ | 18.1 | 2.7 × 10¹² | 16.3 | 1.9 × 10¹⁰ | 8.0 | NM* |
| | MT-4 | 2.9 | 5.4 × 10⁸ | 17.3 | 8.3 × 10¹⁰ | 20.0 | 9.2 × 10¹⁰ | 26.0 | 6.4 × 10⁹ | 26.1 | 7.3 × 10⁹ | 16.0 | 7.9 × 10⁸ |
| | U937 | 2.8 | 8.6 × 10⁷ | 23.0 | 1.8 × 10⁹ | 25.2 | 5.5 × 10⁹ | 28.2 | 6.6 × 10⁹ | 29.1 | 9.8 × 10⁹ | 27.5 | 2.5 × 10⁹ |

NM*—not measured

The same consequences are important for modeling HIV challenging in Hu-SCID mice on PBMC-DC background:

1) Virus strain/isolate multiplication kinetics and animal's challenging timing;
2) The difference between laboratory strain infective activity for cell culture and PBMC substrates.

According to the PBMC-DC background challenging method 4-5 weeks old SCID mice were inoculated intraperitoneally with freshly isolated PBMC, or pre-cultivated for 3 days PBMC, or pre-cultivated for 7 days DC, 5-10×10⁶ one week before the next step. Cells were rinsed twice from human/fetal bovine serum in PBS (phosphate buffer saline) with 10 min spinning at 1200 rpm and then re-suspended in PBS or DMEM without phenol red before the inoculation. The next day after the second PBMC inoculation animals were inoculated intraperitoneally with log phase 10-15×10⁶ cells one of HIV-1 laboratory strains “playbacks” on PBMC or DC passages (FIGS. 17-18). Viral RNA titre in cell material used for challenging should be not less than 10⁷ copies per ml in infective material, it matches to 5×10⁴-10⁵ TCID₅₀ per ml for positive results in challenging (Table 4). Every challenging in vitro material samples are stored for control Real Time PCR measurements (Table 5). Challenges were repeated 2-3 times with different time periods between, SCID mice were supplied with fresh portions of one donor PBMC 5-10×10⁶ cells for each inoculation for one Hu-SCID mouse weekly (one donor's isolation was enough for a group of 5-10 animals).

The term “HIV laboratory strain playback” was created in order to distinguish HIV isolates obtained from HIV-infected individuals and run into cultivation on healthy donor's PBMC in vitro for several days or weeks from characterized HIV laboratory strains that were passed the same healthy donor's PBMC in vitro cultivation for several weekly passages. Thus the term “I-IIV laboratory strain playback” according to the present invention is understood as HIV laboratory strain obtained by culturing a HIV laboratory strain on PBMC cells of a healthy donor for several weeks or passages, in particular for at least 5 weeks passages. Preferred laboratory strains according to the present invention are PokA-79, U455 and H9/IIIB. The variability of envelop proteins sequences for “playbacks” is similar to one of envelop sequences for isolates freshly obtained from HIV infected individuals blood samples.

HIV lab. strain “playbacks” for the current application were obtained as follows: PokA-79, U455 and H9/IIIB strains passaged in vitro on MT-4 cell culture background in log phase were added as 1:20 to pre-cultivated with FHG and cytokine factors at least for 3 days healthy donor's PBMC (in ration 250000 HIV-MT4 infected cells to 5000000 PBMC). Initial in vitro PokA-79-MT4, U455-MT4, IIIB-MT4 titres were 10¹¹-10¹² copies/ml HIV RNA. Virus sorption on PBMC was left for 4 hours incubation in vertical position of culture flasks. Then lymphocyte MT-4 cell culture was discarded from more adhesive (laying on culture flasks plastic surface) PBMC and fresh medium supplied with 1-10% FCS/HS was added for “Playback” passage.

Fresh medium was supplied every 4^{th} day of incubation, new healthy donor pre-cultivated PBMC were added every 7^{th} day. Weekly fresh PBMC supply is named an in vitro passage of HIV strain “playback”.

The current application is summarizing in vivo virus modeling data with laboratory strains U455, 9H/IIIB (FIGS. 13-15) and PokA-79 (FIGS. 4-9) for PBMC background challenging. So infectious source was multiplied and prepared in log phase for inoculation either in MT-4(MT-2), U937 culture or PBMC cells background. Immunity challenging blockage formation requires PBMC in vivo substrate, therefore we run both cell culture and PBMC for control challenging rates tests. The conditions for in vivo virus cultivation are listed in Table 3. These conditions include the presence of 5-15 million “fresh” or infection-free cell culture or PBMC intraperitoneally before challenging (1-2), minimal infectious dose supply for each animal (3), continuous challenging detection period for one donor PBMC material for each animal's treatment (4-7) and matching typical for cell's background in vivo virus cultivation dynamics (8).

TABLE 3
HIV challenging formation conditions in PBMC-,
cell culture background Hu-SCID mice.

| | | Detectable in | |
|---|---|---|---|
| | | Bloodstream HIV | |
| | | RNA Copies/ | |
| | | ml Load | |
| | Applied Treatment (Conditions) for PBMC- | When Treatment is: | |
| | Hu-SCID Mice and cell culture background | | Not |
| ## | HIV Infection Rates in Bloodstream | Applied | Applied |
| 1 | Modest PBMC, DC/PBMC number, 5-15 × 10⁶ | + | − |
| | for one animal | | |
| 2 | Substrate (PBMC, cell culture) cells | + | − |
| | provided within one week before | | |
| | challenging | | |
| 3 | 2-5 × 10⁶ infected cells (TCID₅₀) for | + | − |
| | challenging of one SCID animal | | |
| 4 | One donor PBMC material for each animal | 2-4 weeks | 2-4 |
| | | | weeks |
| 5 | Two or more infected cells inoculations for | 10⁴-10⁸ | 0-10³ |
| | every animal with 2 weeks period between | | |
| | inoculations, viral RNA copies/ml | | |
| 6 | Detectable viral load time period for Hu-SCID | 1-2 weeks | − |
| | mice after the challenging | | |
| 7 | Detectable viral load time period for Hu-SCID | 2-4 weeks | − |
| | mice after the challenging | | |
| 8 | Virus cultivation dynamics typical for cell's | + | − |
| | background in vivo | | |

For the practice application it is worth to have in mind that long-time observation measurements in Hu-SCID blood serum samples are not reasonable in positive controls for vaccine efficiency in vivo assessment. In case challenging is repeated 3-4 times animals often slay within a week after the last administration from total fatigue and exhaust caused by high viral load in their bloodstream like presented on FIG. 13.

It will be understood that different ranges of cell numbers and TCID₅₀ may be used for challenging/engraftment the Hu-SCID mice with HIV. Also, the HIV strains used for this purpose are not limited to a specific strain. Theoretically, all known HIV strains including HIV-1 strains and HIV-2 strains may be used as long as it is made sure that unprotected immunocompetent cells/mice (i.e. cells/mice not vaccinated with the HIV-vaccine to be tested) will be successfully infected with HIV. Preferably the HIV strain for Hu-SCID mice in vivo challenging is selected from the group of highly aggressive breeds with quickly and highly rising in vitro RNA copies/ml titre, such as U455, 9H/IIIB, and PokA-79 (Table 2, FIGS. 7-8, 14-15).

However the problem for successful challenging is the matching between HIV strain envelop proteins variability and immunocompetent cell's CD4 and co-receptors variants presentation. Therefore at first we carried out in vivo minimal infectious dose titration of HIV-1 laboratory strains on lymphocytes MT-4 and monocytes U937 cell culture backgrounds. It occurred that blood serum-detectable challenging can be guaranteed if 2×10⁶ or more infected cells in culture log phase with HIV RNA from 5×10⁷ copies/ml or higher are being used for challenging of one Hu-SCID animal. These numbers are equal to 5×10⁴ TCID₅₀ or higher infection activity measured for in vitro 2-weeks TCID titration per ml for HI-virus laboratory strain on MT-4 or U937 cultures background.

In a preferred embodiment SCID mice are challenged intraperitoneally with e.g. 5-10×10⁶ PBMC or DC cells as background and with 5×10³-10⁵ TCID₅₀ of PBMC-HIV-1 or DC-HIV-1 “playback” strains infectious doses in the same cell culture. The positive results of challenging are presented on FIGS. 17-18.

The activity of in vivo challenging (bloodstream viral load) and in vitro infection titre are very different for HIV laboratory strains multiplying on different backgrounds. First, five in vivo experiments with PokA-79, U455 and H9/IIIB HIV-1 laboratory strains challenging of 34 Hu-SCID mice engrafted with PBMC background carried out in 2009 brought negative results. Second, we tried well-described dendrite cells Hu-SCIDs challenging with the same laboratory strains. Four challenging experiments with PokA-79, U455 and H9/IIIB HIV-1 lab. strains (one group with PokA-79, U455 in vitro DC passages) were carried out on 23 Hu-SCID mice engrafted with DC background in 2010; all results were negative as well (FIG. 18). The third attempt with PBMC-“playbacks” challenging was performed as a conclusion, it occurred to be successful.

In vivo challenging rates (FIGS. 16-17, Table 4) and in vitro multiplication dynamics (FIGS. 14-15, 19-20, Table 5) were analyzed for PokA-79 “playback” strain cultivated on human peripheral blood mononuclear cells (PBMC) and cell cultures background. In vivo PokA-79-PBMC challenging rates on PBMC background were not detectable as HIV RNA copies in animals bloodstream after one single infection's inoculation, achieved 10³ viral RNA copies per ml after two inoculations and 10³-10⁴ viral RNA copies per ml after three infected material inoculations (Table 4, 3^{rd} column, FIG. 17). PokA-79 strain challenging rates for lymphocyte MT-4 in vivo infection modeling were 10²-10³ RNA copies per ml after the first inoculation, 10³-10⁵—after two inoculations and 10⁴-10⁶ HIV RNA copies per ml in bloodstream after three infected material challenges (Table 4, 1^{st} column, FIG. 16). PokA-79 challenging rates for monocyte U937 in vivo infection modeling were 10³ RNA copies per ml after the first inoculation, 10³-10⁵—after two inoculations and 10⁵-10⁷ HIV RNA copies per ml in bloodstream after three infected material challenges (Table 4, 2^{nd} column, FIG. 16). These rates on PBMC background in average were 10²-10⁴ times lower for two and three challenging than the same results obtained for cell culture background (FIG. 17, Table 4). The infection regimes for PBMC background were similar to monocyte-based modeling, so challenging viral RNA rates were detectable in SCID-PBMC mice bloodstream after two-three inoculations when period between challenges was 7 days (Table 4 data, FIGS. 6, 17). If 3 days period was settled between challenges in SCID-PBMC mice like it is being done for higher rates in lymphocyte-background SCID HIV challenging modeling the results in animals bloodstream were not detectable in 45 percent of taken samples.

TABLE 4
PokA-79 strain HIV RNA copies/ml titre in SCID-hu mice bloodstream on PBMC
and cell cultures background in vivo depending on number of challenging.

| | PokA-79 Challenging Viral Load, RNA copies/ml | | | | | | | | |
|---|---|---|---|---|---|---|---|---|---|
| Animal | MT-4 | | | U937 | | | Human PBMC 6^{th} week | | |
| Groups | I | II | III | I | II | III | I | II | III |
| 1 | 9.9 × 10² | 6.9 × 10⁵ | 1.6 × 10⁵ | 6.0 × 10³ | 9.9 × 10³ | 5.2 × 10⁶ | — | 4 × 10² | 5 × 10³ |
| 2 | 2.0 × 10³ | 3.9 × 10⁴ | 9.8 × 10⁶ | 5.7 × 10³ | 3.0 × 10⁵ | 1.2 × 10⁷ | — | — | 9.4 × 10³ |
| 3 | 3.9 × 10³ | 3.9 × 10⁴ | 2.7 × 10⁴ | 2.2 × 10³ | 3.6 × 10⁴ | 4.8 × 10⁵ | — | 5 × 10² | 3.2 × 10⁴ |
| 4 | — | 8.4 × 10³ | 2.6 × 10⁵ | — | 3.0 × 10⁴ | 8.6 × 10⁵ | — | — | 2.8 × 10⁴ |

So, according to in vivo data of the current embodiment, the challenges with HIV may be repeated for a number of times, e.g. for 2-3 times with different time periods in between (FIGS. 16-17). A 3 day period between challenging is preferred for the achievement of the high viral load in a lymphocyte culture background and a 7 day period is preferred for the high viral load in a monocyte culture or PBMC background.

We supposed the lower rates for PBMC-virus “playbacks” challenging background to be in correlation with their in vitro dynamics. In vitro cultivation titres of virus strain on PBMC background were always lower 10²-10⁵ times than on competent for it's cultivation cell cultures background; it was true for both HIV RNA copies per ml Real Time detection and for p24 expression ELISA calibration methods. So, lymphocyte-based PokA-79 cultivation on MT-2 and MT-4 cell lines brings HIV RNA titres to 10¹¹-10¹³, monocyte-based cultivation on U937 cells—to 10⁹ copies/ml RNA (Table 5, FIGS. 14-15). Cultivation on human PBMC for 5-8 weeks brings titre to 10⁷-10⁹ RNA copies/ml (Table 5 line 1, FIG. 7-8, the numbers of infected and fresh cells in culture mixtures were standardized to equal ones). In vitro dynamics of HIV laboratory strain's multiplication for human PBMC background and on DC background (Table 5 lines 2 and 4) was looking like gradual virus dilutions and eradication, curve of HIV RNA copies is dropping down (FIG. 19). It became evident why virus challenging never worked for laboratory strains PBMC and DC background (so called “1^{st} week passages”, FIG. 18).

TABLE 5
PokA-79 laboratory strain and “playbacks” viral replication on PBMC, DC and cell cultures background in vitro.

| | Time of Incubation Period, hours | | | | | | | |
|---|---|---|---|---|---|---|---|---|
| Culture | 0 | 24 | 48 | 72 | 96 | 120 | 144 | 240 |
| background | PokA-79 RNA copies in 1 ml cell suspension in culture media | | | | | | | |
| PBMC 6^{th} | 8.4 × 10⁵ | 6.2 × 10⁶ | 3.1 × 10⁷ | 2.7 × 10⁷ | 5.6 × 10⁷ | 9.3 × 10⁷ | 1.4 × 10⁸ | 2.7 × 10⁸ |
| passage (week) | | | | | | | | |
| PBMC 1^{st} | 5.2 × 10⁸ | 4.3 × 10⁸ | 9.7 × 10⁶ | 3.0 × 10⁶ | 2.8 × 10⁵ | 1.7 × 10⁵ | 8.0 × 10⁴ | — |
| passage (week) | | | | | | | | |
| DC 5^{th} passage | 5 × 10⁴ | 4.5 × 10⁵ | 6.1 × 10⁵ | 2.7 × 10⁵ | 5.4 × 10⁵ | 2.5 × 10⁵ | 4.8 × 10⁵ | 7.0 × 10⁴ |
| (week) | | | | | | | | |
| DC 1^{st} passage | 9.6 × 10⁷ | 1.0 × 10⁶ | 5.5 × 10⁵ | 4.4 × 10⁵ | 8.8 × 10⁴ | 7.3 × 10⁴ | 4.8 × 10⁴ | — |
| (week) | | | | | | | | |
| MT-2 | 7.0 × 10⁷ | 7.5 × 10¹² | 5.4 × 10¹² | 7.9 × 10¹¹ | 7.2 × 10¹⁰ | 8.2 × 10⁸ | NM* | NM* |
| MT-4 | 7.0 × 10⁷ | 8.2 × 10¹² | 2.3 × 10¹³ | 2.7 × 10¹² | 1.9 × 10¹⁰ | 7.9 × 10⁸ | NM* | NM* |
| U937 | 8.6 × 10⁷ | 1.8 × 10⁸ | 5.5 × 10⁹ | 6.6 × 10⁹ | 9.8 × 10⁹ | 2.5 × 10⁹ | NM* | NM* |

PBMC-HIV “playbacks” in vitro titres are similar to monocyte cell culture background: infection titres grow slowly and achieve maximums at 3^{rd}-6^{th} day of cultivation (Table 5 line 1, FIG. 20), whenever lymphocyte-based cultivation brings HIV RNA and p24 titres hit maximums at 1^{st}-2^{nd} day of cultivation and at 3^{rd} day titres of HIV RNA copies start to go down (Table 5 lines 5-7, FIGS. 14-15). However monocyte-based in vitro cultivation viral RNA titres start to drop down after 5^{th} day of cultivation unlike PBMC-based model titres that continue approximately the same levels for 6-10 days of cultivation (Table 5 line 1) and can be maintain with passages on fresh healthy donor pre-cultivated PBMCs weekly.

The most interesting and un-expectable dynamics of HIV strains “playbacks” was observed on dendrite cells (DC) background. In vitro passages on DC made weekly did not have raised virus titres on 5^{th} and further generations, just the opposite, RNA copies continue to be kept on the same levels 10⁴-10⁵ per ml (Table 5 line 3, FIGS. 19-20).

This phenomenon explains why it was not possible to obtain viral load in blood serum of challenged Hu-SCID mice on dendrite cells in vitro passages background (FIG. 20). HIV infectivity potential is determined by envelop gp120's ability to identify and bind CD4 receptor and in some cases co-receptors CXCR4 and CCR5. In case these widely represented in differentiated human tissues receptors are not found on some type of human cells current type of cells cannot be infected with HIV. It is exactly the case with DC that and do not represent as many receptors and other membrane proteins as differentiated tissue cells. In the other words they are median-positioned in development between stem and differentiated cells, therefore they are more “bald” on cell surface and capable of proliferation in vitro and in animal's organism. As it was measured for cell cultures background HIV laboratory strains challenging controls the minimal HIV RNA titre in laboratory strain cultivation material being used for challenging should be 10⁷ copies/ml or higher, and infectious dose measured as TCID₅₀—5×10³ copies/ml or higher. Challenging do not lead to viral load in animals bloodstream from lower titres and TCID₅₀ concentrations in infective material. Consequently, it is expectable that challenging should not work in fresh DC and cultivated DC “playbacks”. Thus, young researcher should not repeat the mistakes that were done for HIV in vivo models challenging.

FIGS. 9-12 demonstrate the microscopy visualization of the difference between infective potential of HIV strains on different backgrounds. FIGS. 9A, 10A, 11A and 12A show intact non-infected cell's cultures MT-4, U937, PBMC and DC, respectively. FIGS. 9B and 10B show lymphocyte MT-4 and monocyte U937 in vitro infection with PokA-79 laboratory strain which is visible as synthythyum formation and cells death on the 3^{rd} day (MT-4) and 5^{th} day of cultivation (U937, proliferation inhibition); synthythyums are marked with white arrows. FIGS. 11B-C present the outlook of PokA79-PBMC and U455-PBMC “playbacks” 6^{th} passage synthythyum formation on 5^{th} day of cultivation. Dendrite cells on FIGS. 12A-B has no difference in morphology without and after infection and no synthythyum formation in infected cells due to low titre and TCID₅₀ values.

In vitro PokA-79 laboratory strain viral replication on PBMC and cell cultures background analysis explains in vivo inoculation regimes and challenging rates for SCID-PBMC HIV infection modeling but it does not reveal the reason why these HIV titre numbers are so very much different for PBMC and cell cultures. Our experience with mass spectrometry analysis of HIV-1 envelop protein variability run for laboratory strains PokA-79, U455 and H9/IIIB and for virus isolates from several cohorts of HIV-1 subtype A infected individuals RF territory-inhabitants [6] brought the only possible conclusion. Laboratory strains selected in cultivation on one or two cell cultures expressing CD4 and/or one of co-receptors CCR5 or CXCR4 in the way how it is normally represented on monoclonal cell culture surface have luck of gp120/gp160 envelop protein's variations. Cell cultures are characterized as monoclonal originated population of human tissue material selected in cultivation in vitro in permanent conditions. Due to monoclonal and constant conditions cultivation cell cultures represent relatively small number of variations of CD4 (CCR5, CXCR4) receptors available for recognition and infection compared to human cells in the bloodstream, PBMC. Therefore the variability of gp120/gp160 for HIV laboratory strains is hundreds or thousands times restricted versus native virus envelop proteins. It means if laboratory strain was put into fresh PBMC environment in vivo or donor PBMC isolates in vitro it has to invade human PBMC instead of cell culture for it\s further multiplication. This viral strain survives with lower titre of infectivity and slower infection development kinetics (Table 5, FIGS. 7, 19) because it's existing gp120/gp160 modifications can recognize only small number of existing variations of CD4/co-receptors on PBMC surface.

TCID₅₀ (tissue cell infectivity dose) is one of the crucial parameters of in vivo HIV challenging because viral load (viral RNA copies number in blood serum or tissue) and virus infective potential are different values in calculation for the same virus particles number. TCID₅₀ is a parameter that characterizes alive and active for new cells invasion virus units. Even in log phase laboratory strain in vitro culture TCID₅₀ referred to the number of HIV viral particles is minimum 100-1000 times lower than total viral particles number, and in overgrown viral in vitro culture where cell deaths are closer to 95-100 percent the difference between TCID₅₀ and RealTime PCR data is 10³-10⁷ in viral particles representation. A simple correlation between viral activity measured in TCID₅₀ and HIV preventive vaccine effectiveness is like this: the higher is TCID₅₀, the lower are chances that HIV-specific immunity can block further challenging effectively. However in reality HIV infection doses transmitted from human to human during sexual contact or IDU (intravenous drug usage) are modest, incomparably lower than ones we run in animal's challenging. Therefore the possibility of HIV infection blockage with specific immune-boost vaccination can be applied into practice.

Infectious doses calculation (Mensch-Reed protocol) in TCID₅₀ units was performed via gradual dilutions method and measured within a period of three weeks in ELISA tests as p24 expression. The infectious dose is correspondent to amount of newly replicated and secreted into cultural media infective virus. All samples were taken from infected cell culture before concentration of material for every animal's challenging and evaluated as average for two similar experiments. TCID₅₀ data for three laboratory strains are presented in Table 6. 3^{rd} and 4^{th} columns contain

TABLE 6
HIV Strains Infectious Doses (TCID₅₀) used for Hu-SCID
challenging on cell culture background.

| | Infected | TCID₅₀ | TCID₅₀ | |
|---|---|---|---|---|
| Laboratory | Culture | in 1 ml | in 1 ml | TCID₅₀ for each |
| Strain | Background | 30 h | 56 h | Hu-SCID Animal |
| U 455 | MT-4 | 2.9 × 10⁷ | — | 5.8 × 10⁶ |
| | MT-2 | 7.2 × 10⁷ | — | — |
| | U937 | 6.9 × 10² | 5.4 × 10⁷ | 1.08 × 10⁶ |
| H9/IIIB | MT-4 | 4.1 × 10⁷ | — | 8.2 × 10⁶ |
| | MT-2 | 9.6 × 10⁷ | — | — |
| | U937 | 7.2 × 10² | 8.2 × 10⁷ | 1.64 × 10⁶ |
| PokA-79 | MT-4 | 6.8 × 10⁶ | — | 1.36 × 10⁶ |
| | MT-2 | 2.9 × 10⁷ | — | — |
| | U937 | 7.1 × 10² | 3.3 × 10⁷ | 6.6 × 10⁵ |

TCID₅₀ data in 1 ml culture suspension for all three laboratory strains on MT-4, MT-2 and U937 cell culture background for 30 and 56 hours of incubation, respectively. 5^{th} column shows TCID₅₀ which was applied to one single animal every inoculation calculated as measured for 30 hours log phase incubation multiplied to 10 ml infected cell culture suspension in average used for concentration of infectious material for one mouse challenging.

TCID₅₀ 30 hours (3^{rd} column) for any of HIV-1 laboratory strains on MT-4 cell culture background varied within 2.9×10⁷-9.6×10⁷ units/ml culture media, TCID₅₀ 56 hours (4^{th} column)—within 6.8×10⁶-9.6×10⁷ units/ml. TCID₅₀ 30 hours (3^{rd} and 4^{th} columns) on U937 cell culture background was 7×10² and for 56 hours—3.3×10⁷-8.2×10⁷. The dynamics of TCID₅₀ increase was the same for U455, H9/IIIB and PokA-79 laboratory strains. These data show that there is a great difference in TCID₅₀ growth between 30 and 56 hours of incubation for lymphocyte cell culture background infection but not much difference in TCID₅₀ growth between 30 and 56 hours of incubation for monocyte U937 cell culture background. It means that maximum TCID₅₀ infectious doses for lymphocyte-based HIV laboratory strains Hu-SCID mice challenging could be achieved after 24-30 hours of culture in vitro incubation, monocyte U937 could be achieved after 48-72 hours of culture in vitro incubation before challenging. So we started to run 30 hours incubation period for MT-4-based challenging protocols and 56 hours for U937-based HIV strains and PBMC-based challenging protocols. These differentiated protocols allow the achievement of higher levels of RNA copies viral load in animal's bloodstream.

Hu-SCID mice can be challenged with 5×10⁴-10⁷ TCID₅₀ of HIV-1 laboratory strain infectious doses in lymphocyte and monocyte culture cells. Suitable cell cultures for this purpose include but are not limited to MT-4 or U937 culture cells. Preferably for successful bloodstream-detectable Hu-SCID mice challenging freshly isolated or pre-cultivated PBMCs as background and 5×10⁴-10⁶ TCID₅₀ of HIV-1 “playback” strain cultivated for 1.5 month on human PBMC are administered to each individual mouse. Suitable HIV-1 or HIV-2 strains for this purpose include but are not limited to U455, H9/IIIB and PokA-79 laboratory strains “playbacks”.

### EXAMPLES

### Example 1

### Identification Of Collected With Antibodies Libraries/Reverse Panning Technique HIV Peptides/Proteins Material, LC-MS-MS (Mass Spectrometry) Data

a) Scaffold software HIV Envelop protein's identification and trustworthy analysis is shown in FIG. 21A)
b) HIV Envelop protein's spectra is shown in FIG. 21B).
c) HIV Envelop protein's triptic peptides mapping is shown in FIG. 21C).
d) Scaffold software HIV Envelop protein's identification and trustworthy analysis is shown in FIG. 21D).
e) HIV Envelop protein's triptic peptides mapping is shown in FIG. 21E).

### Example 2

### Identification of Collected HIV Peptides/Proteins Material, LC-MS-MS (Mass Spectrometry) Data

TABLE 7
The PEAKS analysis software:
Marked protein and peptide details

| | | | InChorus | Coverage | Query | | |
|---|---|---|---|---|---|---|---|
| Accession | ID | Mass | (Score %) | (%) | matched | Description | Marked |
| gi\|62905448 | 5924 | 24174.23 | 29.44 | 3.2 | 1 | envelope glycoprotein [Human | true |
| | | | | | | immunodeficiency virus 1] | |
| gi\|90192283 | 567 | 43626.53 | 4.77 | 1.57 | 55 | envelope glycoprotein [Human | true |
| | | | | | | immunodeficiency virus 1] | |
| gi\|16798231 | 568 | 22471.13 | 3.38 | 2.94 | 36 | envelope glycoprotein [Human | true |
| | | | | | | immunodeficiency virus type 1] | |
| gi\|51848079 | 605 | 15251.62 | 2.95 | 10.22 | 3 | envelope glycoprotein [Human | true |
| | | | | | | immunodeficiency virus 1] | |
| gi\|51537736 | 664 | 17998.39 | 2.79 | 12.5 | 2 | envelope glycoprotein [Human | true |
| | | | | | | immunodeficiency virus 1] | |
| gi\|841085 | 675 | 39568.91 | 2.77 | 3.35 | 3 | envelope glycoprotein, V1-V5 | true |
| | | | | | | region | |
| gi\|1469281 | 677 | 95679.71 | 2.76 | 3.08 | 10 | envelope glycoprotein [Human | true |
| | | | | | | immunodeficiency virus type 1] | |
| gi\|15321690 | 1065 | 12589.34 | 2.7 | 16.81 | 12 | envelope protein [Human | true |
| | | | | | | immunodeficiency virus type 1] | |
| gi\|112378976 | 1118 | 19705.54 | 2.7 | 8.72 | 2 | envelope glycoprotein [Human | true |
| | | | | | | immunodeficiency virus 1] | |
| gi\|164374477 | 1135 | 28011.64 | 2.67 | 4.4 | 2 | envelope glycoprotein [Human | true |
| | | | | | | immunodeficiency virus 1] | |

The HIV Envelop protein's triptic peptides mapping is shown in FIG. 22. The HIV Envelop protein's spectra are shown in FIGS. 23 A) to 23 M)

### Example 3

### Primers Disposition for PCR Products of Immunogenic Composition's Tailoring Mapped for Several Sequences in Alignment is Shown in FIG. 24

### Example 4

### Glycosylated Recombinant Proteins Molecular Weight Calculations

http://www.encorbio.com/protocols/Prot-MW.htm

160 #1 (B.FR.83.HXB2_LAI_IIIB_BRU_K034) There are 856 Amino acids, and MW is 97241.93 Daltons, or 97.24 KiloDaltons; Charge is 30 assuming N and C termini are not blocked

(SEQ ID No. 54)

MRVKEKY QHLWRWGWRW GTMLLGMLMI CSAT-EKLWV
TVYYGVPVWK EATTTLFCAS DAKAYDTEVH NVWATHACVP
TDPNPQEVVL VNVTENFNMW KNDMVEQMHE DIISLWDQSL
KPCVKLTPLC VSLKCTDLKN DTNTNSSSGRMI MEKGEIKNCS
FNISTSIRGK VQKEYAFFYK LDIIPIDNDTTSY KLTSCNTSVI
TQACPKVSFE PIPIHYCAPA GFAILKCNNK TFNGTGPCTN
VSTVQCTHGI RPVVSTQLLL NGSLAE-EEV VIRSVNFTDN
AKTIIVQLNT SVEINCTRPN NNTRKRIRIQ RGPGRAFVTI
GKIGNMRQA HCNISRAKWN NTLKQIASKL REQFGNNKTIIFKQS
SGGDPEIVT HSFNCGGEFF YCNSTQLFNS TWFNSTWSTEGS
NNTEGSDTITLPCR IKQIINMWQK VGKAMYAPPI SGQIRCSSNI
TGLLLTRDGG NSNNESEIFRP GGGDMRDNWR SELYKYKVVK
IEPLGVAPTK AKRRVVQRE KRAV-G-IGA LFLGFLGAAG
STMGAASMTL TVQARQLLSG IVQQQNNLLR AIEAQQHLLQ
LTVWGIKQLQ ARILAVERYL KDQQLLGIWG CSGKLICTTA
VPWNASWSNK SLEQIWNHTT WMEWDREINN YTSLIHSLIE
ESQNQQEKNE QELLELDKWA SLWNWFNITN WLWYIKLFIM
IVGGLVGLRI VFAVLSIVNR VRQGYSPLSF QTHLPTPRGP
DRPEGIEEEG GERDRDRSIR LVNGSLALIW DDLRSLCLFS
YHRLRDLLLI VTRIVELLGR RGW EALKYWWNLL QYWSQELKNS
AVSLLNATAI AVAEGTDRVI EVVQGACRAI RHIPRRIRQG
LERILL

120 #1 There are 511 Amino acids, and MW is 57695.64 Daltons, or 57.70 KiloDaltons

(SEQ ID No. 55)

MRVKEKY QHLWRWGWRW GTMLLGMLMI CSAT-EKLWV
TVYYGVPVWK EATTTLFCAS DAKAYDTEVH NVWATHACVP
TDPNPQEVVL VNVTENFNMW KNDMVEQMHE DIISLWDQSL
KPCVKLTPLC VSLKCTDLKN DTNTNSSSGRMI MEKGEIKNCS
FNISTSIRGK VQKEYAFFYK LDIIPIDNDTTSY KLTSCNTSVI
TQACPKVSFE PIPIHYCAPA GFAILKCNNK TFNGTGPCTN
VSTVQCTHGI RPVVSTQLLL NGSLAE-EEV VIRSVNFTDN
AKTIIVQLNT SVEINCTRPN NNTRKRIRIQ RGPGRAFVTI
GKIGNMRQA HCNISRAKWN NTLKQIASKL REQFGNNKTIIFKQS
SGGDPEIVT HSFNCGGEFF YCNSTQLFNS TWFNSTWSTEGS
NNTEGSDTITLPCR IKQIINMWQK VGKAMYAPPI SGQIRCSSNI
TGLLLTRDGG NSNNESEIFRP GGGDMRDNWR SELYKYKVVK
IEPLGVAPTK AKRRVVQRE KR

41#1 There are 345 Amino acids, and MW is 39564.30 Daltons, or 39.56 KiloDaltons

(SEQ ID No. 56)

AV-G-IGA LFLGFLGAAG STMGAASMTL TVQARQLLSG
IVQQQNNLLR AIEAQQHLLQ LTVWGIKQLQ ARILAVERYL
KDQQLLGIWG CSGKLICTTA VPWNASWSNK SLEQIWNHTT
WMEWDREINN YTSLIHSLIE ESQNQQEKNE QELLELDKWA
SLWNWFNITN WLWYIKLFIM IVGGLVGLRI VFAVLSIVNR
VRQGYSPLSF QTHLPTPRGP DRPEGIEEEG GERDRDRSIR
LVNGSLALIW DDLRSLCLFS YHRLRDLLLI VTRIVELLGR RGW
EALKYWWNLL QYWSQELKNS AVSLLNATAI AVAEGTDRVI
EVVQGACRAI RHIPRRIRQG LERILL

160 #43 (K.CM.96.MP535_AJ249239): There are 842 Amino acids, and MW is 95252.58 Daltons, or 95.25 KiloDaltons Charge is 22 assuming N and C termini are not blocked

(SEQ ID No. 57)

MRVRGMQRNW QTLGNWG ILFLGILII CSNA-DKLWV TVYYGVPVWK
EATPTLFCAS DAKAYEKEVH NVWATHACVP TDPNPQEVEM
ENVTENFNMW KNNMVEQMHT DIISLWDESL KPCVELTPLC
VTLNCTDYKG TNSTN NATSTV VSPAEIKNCS FNITTEIKDK
KKKESALFYR LDVLPLNGEG NNSSTEY RLINCNTSTI
TQTCPKVTFE PIPIHYCAPA GFAILKCKDK RFNGTGPCKN
VSTVQCTHGI KPVVSTQLLL NGSLAE-EEI IIRSENITDN
TKNIIVQLNE TVQINCTRPN NNTRKSIHM GPGKAFYTT
GDIIGDIRQA HCNISGEKWN MTLSRVKEKL KEHFK NGTITFKPP
NPGGDPEILT HMFNCAGEFF YCNTTKLFNE TGENGTITLPCR
IKQIINMWQK VGKAIYAPPI AGSINCSSNI TGMILTRDGG NNTHN
ETFRP GGGDMRDNWR SELYKYKVVQ IEPLGIAPTR ARRRVVQRE
KRAVGLGA [symbol] VFFGFLGAAG STMGAASITL TVQARQLLSG
IVQQQSNLLR AIEAQQHLLQ LTVWGIKQLR ARILAVERYL
KDQQLLGIWG CSGKLICTTN VPWNSSWSNK SWEEIWNNMT
WMEWEKEIGN YSDTIYKLIE ESQTQQEKNE QDLLALDKWA
SLWNWFDITK WLWYIKIFIM IIGGLIGLRI AFAVLSVVNR
VRQGYSPLSF QTLIPTSRGA DRPEGIEEEG GEQDKNRSVR
LVSGFLALAW DDLRNLCLFS YRQLRNLILI VTRILERGLRGGW
EALKYLWNLV QYWSQELKNS AISLLNTTAI AVAGGTDRII
EIGQRAFRAL LHIPRRIRQG LERALL

120 #43: There are 497 Amino acids, and MW is 55884.46 Daltons, or 55.88 KiloDaltons

(SEQ ID No. 58)

MRVRGMQRNW QTLGNWG ILFLGILII CSNA-DKLWV
TVYYGVPVWK EATPTLFCAS DAKAYEKEVH NVWATHACVP
TDPNPQEVEM ENVTENFNMW KNNMVEQMHT DIISLWDESL
KPCVELTPLC VTLNCTDYKG TNSTN NATSTV VSPAEIKNCS
FNITTEIKDK KKKESALFYR LDVLPLNGEG NNSSTEY
RLINCNTSTI TQTCPKVTFE PIPIHYCAPA GFAILKCKDK
RFNGTGPCKN VSTVQCTHGI KPVVSTQLLL NGSLAE-EEI
IIRSENITDN TKNIIVQLNE TVQINCTRPN NNTRKSIHM
GPGKAFYTT GDIIGDIRQA HCNISGEKWN MTLSRVKEKL KEHFK
NGTITFKPP NPGGDPEILT HMFNCAGEFF YCNTTKLFNE
TGENGTITLPCR IKQIINMWQK VGKAIYAPPI AGSINCSSNI
TGMILTRDGG NNTHN ETFRP GGGDMRDNWR SELYKYKVVQ
IEPLGIAPTR ARRRVVQRE KR

41 #43: There are 345 Amino acids, and MW is 39386.14 Daltons, or 39.39 KiloDaltons

(SEQ ID No. 59)

AVGLGA VFFGFLGAAG STMGAASITL TVQARQLLSG IVQQQSNLLR
AIEAQQHLLQ LTVWGIKQLR ARILAVERYL KDQQLLGIWG
CSGKLICTTN VPWNSSWSNK SWEEIWNNMT WMEWEKEIGN
YSDTIYKLIE ESQTQQEKNE QDLLALDKWA SLWNWFDITK
WLWYIKIFIM IIGGLIGLRI AFAVLSVVNR VRQGYSPLSF
QTLIPTSRGA DRPEGIEEEG GEQDKNRSVR LVSGFLALAW
DDLRNLCLFS YRQLRNLILI VTRILERGLRGGW EALKYLWNLV
QYWSQELKNS AISLLNTTAI AVAGGTDRII EIGQRAFRAL
LHIPRRIRQG LERALL

160 #6 (our A1.RU.03.03RU20_{—}06_{—}13_AY500393) There are 805 Amino acids, and MW is 90669.32 Daltons, or 90.67 KiloDaltons: Charge is 24 assuming N and C termini are not blocked

(SEQ ID No. 60)

MKAKGMQRNY QHLWRWG--- -XMLFWXIIM CKAA-ENLWV
TVYYGVPVWR DAETTLFCAS DAKAYDKEVH NVWATHACVP
TDPNPQEIAL ENVTEKFDMW KNNMVEQMQT DIISLWDQSL
KPCVKLTPLC VTLNCAEPNS TSSNNNS--- ---------- --
-VNSNSSD SVFEEMKNCT FNMTTELRDK RKTVHSLFYK
LDIVSTGS-- --NGS----- ---- ---GQY RLINCNTSAM
TQACPKVTFE PIPIHYCAPA GFAILKCKDT NFTGTGPCKN
VSTVQCTHGI KPVVSTQLLL NGSLAE-KEV MXRSENITDN
GKXIIVQLTE PVNITCIRPG NNTRTSIRI- -GPGQTFYAT
XDVIGDIRKA YCXVSRAAWX STLQKISTQL R--K-YFN--
NKT-IXFKNS S-GGDLEVTT HSFNCGGEFF YCNTTDLFNS
TW----DGXG TXT------- XITXA----N GT--ITLPCR
IKQIINMWQR VGQAMYAPPIKGSIRCESNI TGLXLTRDGG
GGT--NXX-- ----XETFRP IGGNMRDNWR SELYKYKVVK
IEPIGVAPTR AKRRVV-ERE KR AI-G-LGA AFLGFLGAAG
STMGAASMTL TVQARQLLSG IVQQQSNLLR AIEAQQHLLK
LTVWGIKQLQ ARVLAVERYL KDQQLLGIWG CSGKLICTTN
VPWNSSWSNK SQSEIWDNMT WMQWDKEVIN YTXIIYDLIE
KSQNQQEKNE QDLLALDKWA SLWXWFDISN WLWYIKIFII
IVGSLIGLRI XFAVLYIINR ARQGYSPLSL QTLTPHPEGP
DRPGRIKEEG GEQGRDRSIR LVSGFLALAW DDLRSLCLFS
YHRLRDFIXI AARTVELLGR SSLKGLRLGW EGLKYLGNLL
GYWGQELKSS AINLIDTIAI

120 #6 There are 493 Amino acids, and MW is 55328.29 Daltons, or 55.33 KiloDaltons

(SEQ ID No. 61)

MKAKGMQRNY QHLWRWG--- -XMLFWXIIM CKAA-ENLWV
TVYYGVPVWR DAETTLFCAS DAKAYDKEVH NVWATHACVP
TDPNPQEIAL ENVTEKFDMW KNNMVEQMQT DIISLWDQSL
KPCVKLTPLC VTLNCAEPNS TSSNNNS--- ---------- --
-VNSNSSD SVFEEMKNCT FNMTTELRDK RKTVHSLFYK
LDIVSTGS-- --NGS----- ---- ---GQY RLINCNTSAM
TQACPKVTFE PIPIHYCAPA GFAILKCKDT NFTGTGPCKN
VSTVQCTHGI KPVVSTQLLL NGSLAE-KEV MXRSENITDN
GKXIIVQLTE PVNITCIRPG NNTRTSIRI- -GPGQTFYAT
XDVIGDIRKA YCXVSRAAWX STLQKISTQL R--K-YFN--
NKT-IXFKNS S-GGDLEVTT HSFNCGGEFF YCNTTDLFNS
TW----DGXG TXT------- XITXA----N GT--ITLPCR
IKQIINMWQR VGQAMYAPPIKGSIRCESNI TGLXLTRDGG
GGT--NXX-- ----XETFRP IGGNMRDNWR SELYKYKVVK
IEPIGVAPTR AKRRVV-ERE KR

41#1 There are 346 Amino acids, and MW is 39333.71 Daltons, or 39.33 KiloDaltons

(SEQ ID No. 62)

AI-G-LGA AFLGFLGAAG STMGAASMTL TVQARQLLSG
IVQQQSNLLR AIEAQQHLLK LTVWGIKQLQ ARVLAVERYL
KDQQLLGIWG CSGKLICTTN VPWNSSWSNK SQSEIWDNMT
WMQWDKEVIN YTXIIYDLIE KSQNQQEKNE QDLLALDKWA
SLWXWFDISN WLWYIKIFII IVGSLIGLRI XFAVLYIINR
ARQGYSPLSL QTLTPHPEGP DRPGRIKEEG GEQGRDRSIR
LVSGFLALAW DDLRSLCLFS YHRLRDFIXI AARTVELLGR
SSLKGLRLGW EGLKYLGNLL GYWGQELKSS AINLIDTIAI
AVARWTDXVI EIGQRLCRAI RNIPRRIRQG XEKALQ

160 #26 (C.ZA.01.01ZATM45_AY228557) There are 878 Amino acids, and MW is 99122.18 Daltons, or 99.12 KiloDaltons; Charge is 15.5 assuming N and C termini are not blocked

(SEQ ID No. 63)

MRVRGIPRNW QQWWIWI--- -ILGFWMLLI CNVG-GNSWV
TIYYGVPVWR EAKTTLFCAS DAKAHETEVH NVWATHACVP
TDPNPQEIEL ENVTENFNMW KNDMVDQMHE DIISLWDQSL
KPCVKLTPLC VTLNCTNATR PVTRTNTTAT GTNNTVTNCS
GSASTNNTCM ENIEGMKNCS FNITTELRDK KKKEYALFYR
LDIVPLNETR D-NSS----- ---------Y RLINCNTSTI
TQACPKVSFD PIPIHYCAPA GYAILKCNNK TFSGTGPCNN
VSTVQCTHGI MPVVSTQLLL NGSLAE-EMV IIRSDNMTNA
ATTIIVHLKD PVEIVCTRPN NTTRREVGI- -GPGQTFYTT
GQIIGDIRQA HCNITGKEWN KTLRQVGAEL E--K-HFP--
NKT-IQFKPH S- GGDLEITT HSFTCSGEFF YCNTSKLFNI
SMS---NLT- ---Y------ NNTDNTD--N PTQ- ITLPCR
IKQIINMWQE VGRAIYAPPI AGNITCKSNI TGLLLTWDGG
SGE--NNT-- ----- ETFRP GGGDMRDNWR SELYKYKVVE
IKPLGIAPTE AKRRVV-ERE KR AI-G-IGA VFLGFLGAAG
STMGAASITL TVQARQLLSG IVQQQSNLLR AIEAQQHLLQ
LTVWGIKQLQ ARVLAIERYL KDQQLLGIWG CSGKLICTTS
VPWNSSWSNK TLGEIWNNMT WMEWDKEISN YTHTIYQLLE
ESQIQQEQNE KELLALDSWK NLWNWFDISN WLWYIKIFIM
IVGGLIGLRI VFAVLSIVNR VRQGYSPLSF QTLTPSPREP
DRLRGIEEEG GEQDKGRSIR LVQGFLALAW DDLRSLCLFS
YHRLRDFISI AARVVEVLGH SSLRGLQRGW EALKYLKSLV
QYWGLELKKS AVSLLDTLAI AVGEGTDRII ELIQGICRAI
RNIPRRIRQG FEAALL

120 #26 (C.ZA.01.01ZATM45_AY228557) There are 526 Amino acids, and MW is 59182.32 Daltons, or 59.18 KiloDaltons

(SEQ ID No. 64)

MRVRGIPRNW QQWWIWI--- -ILGFWMLLI CNVG-GNSWV
TIYYGVPVWR EAKTTLFCAS DAKAHETEVH NVWATHACVP
TDPNPQEIEL ENVTENFNMW KNDMVDQMHE DIISLWDQSL
KPCVKLTPLC VTLNCTNATR PVTRTNTTAT GTNNTVTNCS
GSASTNNTCM ENIEGMKNCS FNITTELRDK KKKEYALFYR
LDIVPLNETR D-NSS----- ---------Y RLINCNTSTI
TQACPKVSFD PIPIHYCAPA GYAILKCNNK TFSGTGPCNN
VSTVQCTHGI MPVVSTQLLL NGSLAE-EMV IIRSDNMTNA
ATTIIVHLKD PVEIVCTRPN NTTRREVGI- -GPGQTFYTT
GQIIGDIRQA HCNITGKEWN KTLRQVGAEL E--K-HFP--
NKT-IQFKPH S- GGDLEITT HSFTCSGEFF YCNTSKLFNI
SMS---NLT- ---Y------ NNTDNTD--N PTQ- ITLPCR
IKQIINMWQE VGRAIYAPPI AGNITCKSNI TGLLLTWDGG
SGE--NNT-- ----- ETFRP GGGDMRDNWR SELYKYKVVE
IKPLGIAPTE AKRRVV-ERE KR

41 #26 (C.ZA.01.01ZATM45_AY228557) There are 352 Amino acids, and MW is 39957.88 Daltons, or 39.96 KiloDaltons

(SEQ ID No. 65)

AI-G-IGA VFLGFLGAAG STMGAASITL TVQARQLLSG
IVQQQSNLLR AIEAQQHLLQ LTVWGIKQLQ ARVLAIERYL
KDQQLLGIWG CSGKLICTTS VPWNSSWSNK TLGEIWNNMT
WMEWDKEISN YTHTIYQLLE ESQIQQEQNE KELLALDSWK
NLWNWFDISN WLWYIKIFIM IVGGLIGLRI VFAVLSIVNR
VRQGYSPLSF QTLTPSPREP DRLRGIEEEG GEQDKGRSIR
LVQGFLALAW DDLRSLCLFS YHRLRDFISI AARVVEVLGH
SSLRGLQRGW EALKYLKSLV QYWGLELKKS AVSLLDTLAI
AVGEGTDRII ELIQGICRAI RNIPRRIRQG FEAALL

### Example 5

### Vaccination with PokA-79-PBMC Strain Envelop Protein Cocktails and Challenging with PokA-79-PBMC in SCID-PBMC Mice

### Example 5A

Immune responses for gp160-1 and gp160-2 in challenged with PokA-79-PBMC strain SCID-PBMC Mice. The result is shown in FIG. 25 A).

### Example 5B

Recombinant gp160-1 and gp160-2 immunizations effect for SCID-PBMC challenging with PokA-79-PBMC 6th passage strain. The result is shown in FIG. 25 B).

### Example 5C

Recombinant gp160-PokA-PBMC and gp160-U455-PBMC immunization effect for challenging with PokA79-PBMC and U455-PBMC 6-8 weeks strains. The result is shown in FIG. 25 C).

Immunization effect tests were completed for HIV subtype A laboratory strain's PokA-79 and U455 “playback” cultivated in vitro on isolated healthy donor PBMC for 5-6 weeks. The application of principle of mass spectrometry analysis of represented in majority HIV-1 gp120/gp160 epitopes for vaccine development brought the material for SCID-PBMC immunizations. These immunizations occurred to be able to prevent or suppress further animal's challenging with the same laboratory HIV strain which aminoacid sequences recognitions were used for design of primers for major gp120/gp160 epitope's cloning and expression.

The groups of animals were treated as follows: 4-5 weeks old SCID mice were inoculated with 5-10×10⁶ of pre-cultivated for 3 days in presence of FHG and IL-2 healthy donor PBMC intraperitoneally once a week. Two weeks later the 1^{st} PBMC inoculation mice were divided into groups (3 animals in every group) and immunized subcutaneously with peptides mixture gp160-1 (PokA79-PBMC) or gp160-2 (U455-PBMC). Each animal received 25 mkg proteins in cocktail mixed in 200 mkl incomplete Freund adjuvant for 1 hour at +4 C. Immunization was repeated three times with the between period of two weeks. Immune response was tested in ELISA assays (FIG. 25A). Three days after the last immunization and the scheduled PBMC inoculation animals were challenged intraperitoneally with log phase 5×10⁶ PokA-79-PBMC (FIG. 25 B), PokA-79-PBMC and U455-PBMC “playbacks” (FIG. 25 C). Viral RNA titre in cell material used for challenging should be minimum 5×10⁷ copies per ml, preferably not less than 10¹⁰ copies per ml; in vitro material controls were stored for Real Time PCR measurements. The blood of the same donor was used for PBMC isolation for mice engraftment and “playbacks” cultivation. Control Hu-SCID mice were challenged intraperitoneally with the same amount of PokA-79-PBMC and U455-PBMC “playbacks” in the same time with experimental groups using background PBMC engraftment but without previous immunizations. Challenges were completed 2 times with three days periods between, Hu-SCID mice were provided with fresh portions of PBMC weekly. Animals were sacrificed one week after the second challenging and blood serum samples were taken for ELISA and Real Time PCR measurements.

There were three groups of SCID-PBMC immunized with gp160-1 and four groups immunized with gp160-2; serums of BalbC mice immunized with the same cocktails gp160-1 and gp160-2 were taken as controls for immunization results detection (FIG. 25 A, B). In mice serums diluted 4-14 times with quantitative calibration curve gp160-1 and gp160-2 antibodies titre were detectable for all SCID-PBMC animals, however control BalbC mice HIV env-specific antibodies titre were always higher (approximately 2-2.5 times in ELISA test) than SCID-PBMC animals titre. Gp160-1 and gp160-2 immune responses assessment were positive for immunized SCID-PBMC mice for at least 2 weeks after the last immunization (FIG. 25 A).

HIV titre in bloodstream measured with Real Time PCR was not detectable in 4 groups immunized with gp160-2. In one of 3 groups immunized with gp160-1 two animals provided challenging rate 400 and 500 HIV RNA copies/ml (FIG. 25 B). 3rd group's viral load “leakage” (Positive PokA-79 viral RNA titre in bloodstream, FIG. 25 B) correlates with the lowest ELISA gp160-1 antibodies titre evaluated for the same SCID-PBMC-gp160-1 animal's group (FIG. 25 A). SCID-PBMC mice challenged with PokA-79-PBMC “playback” without immunization pre-treatment were tested as positive controls (FIG. 25 B, C). Challenging did not work in 2 animals from different groups for both “playbacks”, we suppose it can happen in case of alterations in immune deficiency status of SCID animals. However these results are normal deviation for group's statistic analysis. Both PokA-79-PBMC and U455-PBMC “playbacks” proteins cocktails immunizations led to diminishing number of positive challenging cases (FIG. 25 B) or to blockade of challenging in Hu-SCID animals engrafted with “playbacks” of the same HIV strain (FIG. 25 C).

### REFERENCES

1. Bankert R B, Chen Fang—An “SCID Mouse Having a Normal Immune Response to Exogenous Antigens” Patent EP0937389 (A2), C12N5/00; A01K67/027, 1999 Aug. 25;
2. Bonyhadi M L, Kaneshima H. “The Hu-SCID Mouse: an in vivo Model for HIV Infection in Humans” Molecular medicine today, 1997 June, pp 246-253;
3. Brainard D. M, Seung E., Frahm N., Cariappa A., Bailey C. C. et al. “Induction of Robust Cellular and Humoral Virus-Specific Adaptive Immune Responses in Human Immunodeficiency Virus-Infected Humanized BLT Mice”, J. Virology, 2009 July; 83(14): 7305-7321;
4. Brams P. Morrow P R “Neutralizing high affinity human monoclonal antibodies specific to RSV F-protein and methods for their manufacture and therapeutic use thereof” Patent US2002001798 (A1), C07K16/00; C07K16/10, 2002 Jan. 3;
5. Capodici J. et al., “Large-Scale Beta-Propiolactone Inactivation of HIV for Vaccines” Bioprocess Int., 2006, February, pp 36-41;
6. Coccia M A, Brams P. “Methods for Producing Human Antibodies in SCID Mice Using Dendrite Cells” WO Patent #WO9912553 (A1), C07K16/40, A61K39/00; 1999 Mar. 18;
7. Filinova E. <<HIV preventive vaccine based on HIV specific antibodies>> Patent WO2009046984 (A1), US20070978536P 20071009, C12N15/10, A61K39/21, 2009 Apr. 16;
8. Franchini G., Hel Z., Pavlakis G. et al. “Improved immunogenicity using a combination of DNA and Vaccinia virus vaccines” Appl.: The Government of USA, A61K 39/21 (2006 January), WO/2001/082964, Aug. 11, 2001;
9. Friedman H., Specter S., Bendinelli M. “In vivo Models of HIV Disease and Control” Springer, 2006, Chapter 2: Anderson E. R. at al. “Animal Model Systems of HIV-Diseases”; Chapter 4: Laperanta K. et al. “SCID Mice Transplanted with Human Cells as Small Animal Models in AIDS Research”;
10. Ito M., Kobayashi K., Nakahata T. et al. “Method of producing a mouse suitable for engraftment, differentiation and proliferation of heterologous cells, mouse produced by this method and use of the mouse” Patent US2007011753 (A1), 2007 Jan. 11;
11. Karp N. M. “Immunogenic Composition and Method of Developing a Vaccine Based on Psoralen Inactivated HIV” Patent WO2005040353, 2005 May 6;
12. Lapenta C, Fais S, Rizza P, Spada M, Logozzi M A et al. “U937-SCID mouse xenografts: a new model for acute in vivo HIV-1 infection suitable to test antiviral strategies” Antiviral Research 36 (1997) 81-90;
13. Mosier D E, Gulizia R J, Baird S M, Wilson D B. “Transfer of a functional human immune system to mice with severe combined immunodeficiency” Nature, 1988 Sep. 15; 335(6187):256-9;
14. Picker, L. J., Jarvis, M., Nelson, J, A. “SIV and HIV vaccination using RHCMV- and HCMV-based vaccine vectors” US Patent C12N 15/63 (2006.01), Appl.: Oregon Health and Science University, WO/2006/031264, 23 Mar. 2006;
15. Poignard P. et. al., “Heterogeneity of envelope molecules expressed on primary human immunodeficiency virus type 1 particles as probed by the binding of neutralizing and non-neutralizing antibodies” J. Virol. 2003. January; 77.(1.): 353-65;
16. Sehon A, Bitoh S. “Testing Individual Immune Response to an Antibody with SCID Mouse” Patent WO9405779 (A1), A01K67/027; A01K67/027; 1994 Mar. 17;
17. Steinbrook R. “One Step Forward, Two Steps Back—Will There Ever Be an AIDS Vaccine?” New England Journal of Medicine, Dec. 27, 2007 (26), 357:2653-2655;
18. Stoddart C. A, Bales C. A, Bare J. C, Chkhenkeli G., Galkina S. A. et al. “Validation of the Hu-SCID Thy/Liv Mouse Model with Four Classes of Licensed Antiretrovirals”, PLoS August 2007 Issue 8 p655;
19. Su L., Kaneshima H. Bonyhadi M., Salimi S., Kraft D., Rabin L., and McCune J. M. “HIV-1-Induced Thymocyte Depletion Is Associated with Indirect Cytopathicity and Infection of Progenitor dells In Vivo”, Immunity, January 1995, V. 2, pp 25-36;
20. Uckun F M “Pokeweed Antiviral Protein Polypeptides with Antiviral Activity” Patent WO03106479 (A2), C12N15/09; A61K38/00, 2003 Dec. 24;
21. William C. Griffin W C, Middaugh L D, Cook J E, Tyor W R “The severe combined immunodeficient (SCID) mouse model of human immunodeficiency virus encephalitis: Deficits in cognitive function” Journal of Neurovirology, Volume 10, Issue 2 Apr. 2004, pp 109-115;
22. Van Duyne R, Pedati C, Guendel I., Carpio L, Kehn-Hall K, Saifuddin M, Kashanchi F. “The utilization of humanized mouse models for the study of human retroviral infections” Retrovirology 2009, 6:76;
23. Fikhtengholtz G. M. “Differential and Integral Math Analysis”, 7^{th} edition, volume2, Moscow, 1969, *in Russian;
24. Wolkenstein M. V. “Molecular Biophysics”, Moscow, “Nauka”, 1975, * in Russian.
25. Jonuleit H, Kuhn U, Muller G, Steinbrink K, Paragnik L, Schmitt E, Knop J, Enk A H.: Pro-inflammatory cytokines and prostaglandins induce maturation of potent immunostimulatory dendrite cells under fetal calf serum-free conditions. Eur J Immunol. 1997 December; 27(12):3135-42.

1. A method for producing an HIV vaccine composition, comprising the steps of:
a) creation of a library comprising HIV-1 specific antibodies,
b) enrichment for HIV-1-specific antibodies in the library by panning with HIV-1 peptides, in particular native and/or recombinant HIV-1 peptides,
c) multiplying HIV-1 material comprising HIV-1 peptides, polypeptides or proteins,
d) collecting HIV-1 peptides of the multiplied HIV-1 material using HIV-1-specific antibodies of step b) bound to a support,
e) identification and characterization of the HIV-1 peptides obtained in step d) by mass spectrometry, in particular by MS-MS,
f) cloning of fragments of the gp120 gp160 genes encoding the peptides identified in step e),
g) expressing glycosylated env HIV-1 peptides using the results of step f) in a eukaryotic expression system,
h) purification of the glycosylated env HIV-1 peptides, and
i) production of a vaccine composition,
characterized in that for cloning and/or expressing glycosylated env HIV-1 peptides in step f) and/or g), at least one primer selected from the group consisting of:
(i) V1 forward for subtypes A, G B, C, F 1, H:
(SEQ ID No. 1)
5′-CTC TGY GTY ACT TTA XXX XXX XXX-3′,
(ii) V2 forward for all subtypes:
(SEQ ID No. 3)
5′-AAA ACT GCT CTT WCA XXX XXX XXX-3′,
(iii) V3 forward for A, B, G subtypes:
(SEQ ID No. 5)
5′-TAV AAA TTA ATT GTA XXX XXX XXX X-3′,
(iv) V3 forward For subtype D:
(SEQ ID No. 7)
5′-TAV CAA TTA ATT GCA XXX XXX XXX X-3′,
(v) V4 forward for all subtypes:
(SEQ ID No. 9)
5′-GAA TTT TTC TAT TGY AAXXX XXX XXX-3′,
(vi) V5 forward for A, B, D, G subtypes:
(SEQ ID No. 11)
5′-ACA AGA GAT GGT GGX XXX XXX X-3′,
and
(vii) V5 forward for subtype C:
(SEQ ID No. 13)
5′-ACA CGT GAT GGA GGX XXX XXX X-3′,
and at least one primer selected from the group consisting of:
(viii) gp41(160) Reverse for A, B, D subtypes:
(SEQ ID No. 46)
ATA GCG GCC GCC TAG TGG TGG TGA TGG TGG TGT AG YAA
AGC YCT TTC NAA GCC CTG TC,
(ix) a primer derivative of (viii) lacking the sequence encoding a His-Tag, and/or comprising a sequence encoding a tag,
(x) gp41(160) Reverse for subtype A, rare variant:
(SEQ ID No. 66)
ATA GCG GCC GCC TAG TGG TGG TGA TGG TGG TGT AGC
AAA GCY CTT TCN GCG CCC TGT C,
(xi) gp41(160) Reverse for C subtype:
(SEQ ID No. 67)
ATA GCG GCC GCC TAG TGG TGG TGA TGG TGG TGT WGC
AAA GCT GCT TCA AAG CCC TGT C,
(xii) gp41(160) Reverse for G subtype:
(SEQ ID No. 68)
ATA GCG GCC GCC TAG TGG TGG TGA TGG TGG TGT AGC
AAA GCY CTT TCN AAG CCT TGT C,
(xiii) gp120 Reverse Const5 for all subtypes:
(SEQ ID No. 52)
5′-ATA GCG GCC GCC TAG TGG TGG TGA TGG TGG TGT CTT
TTT TCT CTY TSC ACC ACT CTY CT-3′,
and
(xiiii) a primer derivative of (viii) lacking the sequence encoding a His-Tag, and/or comprising a sequence encoding a tag,
are used.

2. The method according to claim 1, wherein 2, 3, 4, 5, 6, 7, 8, 9, 10, 12, 15, 18 or more variants of primer (i), (ii), (iii), (iv), (v), (vi) and/or (vii) are used, wherein the variants differ in the variables “X”.

3. The method according to claim 1 or 2, wherein the primers (i) to (vii), and (x) to (xii) and one of (xiii) and (xiiii) and one of (viii) and (ix) are used.

4. A method for producing an HIV vaccine composition, comprising the steps of:
a) creation of a library comprising HIV-1 specific antibodies,
b) enrichment for HIV-1-specific antibodies in the library by panning with HIV-1 peptides, in particular native and/or recombinant HIV-1 peptides,
c) multiplying HIV-1 material comprising HIV-1 peptides, polypeptides or proteins,
d) collecting HIV-1 peptides of the multiplied HIV-1 material using HIV-1-specific antibodies of step b) bound to a support,
e) identification and characterization of the HIV-1 peptides obtained in step d) by mass spectrometry, in particular by MS-MS,
f) cloning of fragments of the gp120 gp160 genes encoding the peptides identified in step e),
g) expressing glycosylated env HIV-1 peptides using the results of step f) in a eukaryotic expression system,
h) purification of the glycosylated env HIV-1 peptides, and
i) production of a vaccine composition,
characterized in that for cloning and/or expressing glycosylated env HIV-1 peptides in step and/or g), at least one primer selected from the group consisting of:
(i) Forward gp120 Const1 for A subtype:
5′-AAT TCT AGA CRC TRC AGA AAA CTT GTG GGT YAC-3′(SEQ ID No. 52), or a derivative thereof lacking the XbaI site, and/or comprising a restriction site at the 5′ end,
(ii) Forward gp120 Const1 for B subtype:
5′-AAT TCT AGA CGC TRC AGA AMA ATT GTG GGT CAC-3′ (SEQ ID No. 69), or a derivative thereof lacking the XbaI site, and/or comprising a restriction site at the 5′ end,
(iii) Forward gp120 Const1 for C subtype:
5′-AAT TCT AGA CGT RRT GGG RAA CTT GTG GGT CAC-3′ (SEQ ID No. 70), or a derivative thereof lacking the XbaI site, and/or comprising a restriction site at the 5′ end, and
(iv) Forward gp120 Const1 for G subtype:
5′-AAT TCT AGA CGC CTC ARA TAA CTT GTG GGT CAC AG-3′(SEQ ID No. 71), or a derivative thereof lacking the XbaI site, and/or comprising a restriction site at the 5′ end,
and at least one primer selected from the group consisting of:
(v) V1 reverse for all subtypes:
(SEQ ID No. 25)
5′-GCA GTT TTT YAT TTC TYX XXX XXX XXX-3′,
(vi) V2 reverse for subtypes A, D, C:
(SEQ ID No. 28)
5′-AGG TAT TRC AAT TTA TTX XXX XXX X-3′,
(vii) V2 reverse for subtype B:
(SEQ ID No. 29)
5′-CTG AGG TRT TAC AAX XXX XXX X-3′,
(viii) V2 reverse for subtype G:
(SEQ ID No. 30)
5′-AGA CAT TAC AAT TTA TTX XXX XXX X-3′,
(ix) V2 reverse for subtype F1:
(SEQ ID No. 31)
5′-TTG AGG TAT TRC AAX XXX XXX X-3′,
(x) V3 reverse for subtypes A, D, (C):
(SEQ ID No. 40)
5′-AAA GTT TBA TTC CAX XXX XXX XX-3′,
(xi) V3 reverse for subtype B:
(SEQ ID No. 41)
5′-AAA GTG TTR TTC CAX XXX XXX XX-3′,
(xii) V4 reverse for subtype G:
(SEQ ID No. 19)
5′-CAA TTT GTT TTA TYY TAC A XX XXX XXX X-3′,
(xiii) V4 reverse for subtypes A, B, C, D:
(SEQ ID No. 20)
5′-TAA TTT GYT TTA TTY TGC A XX XXX XXX X-3′,
(xiv) V5 reverse for all subtypes:
(SEQ ID No. 15)
5′-TCC TCC TSC AGG TCT GAA XXX XXX XXX X-3′,,
(xv) gp41(160) Reverse for A, B, D subtypes:
(SEQ ID No. 46)
ATA GCG GCC GCC TAG TGG TGG TGA TGG TGG TGT AG YAA
AGC YCT TTC NAA GCC CTG TC,
(xvi) a primer derivative of (viii) lacking the sequence encoding a His-Tag, and/or comprising a sequence encoding a tag,
(xvii) gp41(160) Reverse for subtype A, rare variant:
(SEQ ID No. 66)
ATA GCG GCC GCC TAG TGG TGG TGA TGG TGG TGT AGC
AAA GCY CTT TCN GCG CCC TGT C,
(xviii) gp41(160) Reverse for C subtype:
(SEQ ID No. 67)
ATA GCG GCC GCC TAG TGG TGG TGA TGG TGG TGT WGC
AAA GCT GCT TCA AAG CCC TGT C,
and
(xix) gp41(160) Reverse for G subtype:
(SEQ ID No. 68)
ATA GCG GCC GCC TAG TGG TGG TGA TGG TGG TGT AGC
AAA GCY CTT TCN AAG CCT TGT C,
are used.

5. The method according to claim 4, wherein 1, 2, 3, 4, 5, 6, 7, 8, 9, 10, 12, 15, 18, or more variants of primer (v), (vi), (vii), (viii), (ix), (x), (xi), (xii), (xiii) and/or (xiv) are used, wherein the variants differ in the variables, in particular the variables “X”.

6. The method according to claim 4 or 5, wherein the primers (i) to (xiv), and (xvii) to (xix) and one of (xv) and (xvi) are used.

7. A method for producing an HIV vaccine composition, comprising the steps of:
a) creation of a library comprising HIV-1 specific antibodies,
b) enrichment for HIV-1-specific antibodies in the library by panning with HIV-1 peptides, in particular native and/or recombinant HIV-1 peptides,
c) multiplying HIV-1 material comprising HIV-1 peptides, polypeptides or proteins,
d) collecting HIV-1 peptides of the multiplied HIV-1 material using HIV-1-specific antibodies of step b) bound to a support,
e) identification and characterization of the HIV-1 peptides obtained in step d) by mass spectrometry, in particular by MS-MS,
f) cloning of fragments of the gp120 gp160 genes encoding the peptides identified in step e),
g) expressing glycosylated env HIV-1 peptides using the results of step f) in a eukaryotic expression system,
h) purification of the glycosylated env HIV-1 peptides, and
i) production of a vaccine composition,
wherein the glycosylated env HIV-1 peptides in step g) are characterized by:
(i) the gp120 peptides have a length of 441-541 amino acids and/or a glycosylated protein molecular weight of about 90-115 kDa, and
(ii) the gp160 peptides have a length of 816-923 aminoacids, and/or a glycosylated protein molecular weight of about 145-175 kDa, and
(iii) at least 50, preferably at least 70, more preferably at least 100, even more preferably at least 150 different HIV-1 envelop peptides are expressed.

8. A method according to claim 7, wherein the vaccine composition of step i) comprises
(a) at least one peptide characterized by the N-terminal sequence LCVTL (SEQ ID No. 72)
(b) at least one peptide characterized by the N-terminal sequence NCSX, wherein X is Y or F (SEQ ID No. 73)
(c) at least one peptide characterized by the N-terminal sequence XINC, wherein X is K or Q or E (SEQ ID No. 74),
(d) at least one peptide characterized by the N-terminal sequence XINC, wherein X is T or P or A (SEQ ID No. 75),
(e) at least one peptide characterized by the N-terminal sequence EFFYC (SEQ ID No. 76),
(f) at least one peptide characterized by the N-terminal sequence TRDG (SEQ ID No. 77),
and
(g) at least one peptide characterized by the N-terminal sequence LDXXENLWVLD, wherein X is T or A, independently from each other (SEQ ID No. 78).

9. A HIV vaccine composition, obtainable by a method according to any of claims 1 to 8.

10. A HIV vaccine composition comprising at least 50, preferably at least 70, more preferably at least 100, even more preferably at least 150 different HIV-1 envelop peptides, characterized in that:
(i) the gp120 peptides have a length of 441-541 amino acids and/or a glycosylated protein molecular weight of about 90-115 kDa, and
(ii) the gp160 peptides have a length of 816-923 amino acids, and/or a glycosylated protein molecular weight of about 145-175 kDa.

11. The vaccine composition of claim 10, further comprising:
(a) at least one peptide characterized by the N-terminal sequence LCVTL (SEQ ID No. 72)
(b) at least one peptide characterized by the N-terminal sequence NCSX, wherein X is Y or F (SEQ ID No. 73)
(c) at least one peptide characterized by the N-terminal sequence XINC, wherein X is K or Q or E (SEQ ID No. 74),
(d) at least one peptide characterized by the N-terminal sequence XINC, wherein X is T or P or A (SEQ ID No. 75),
(e) at least one peptide characterized by the N-terminal sequence EFFYC(SEQ ID No. 76),
(f) at least one peptide characterized by the N-terminal sequence TRDG (SEQ ID No. 77),
and
(g) at least one peptide characterized by the N-terminal sequence LDXXENLWVLD, wherein X is T or A, independently from each other (SEQ ID No. 78).

12. A composition comprising at least one of following nucleic acids:
(i) V1 forward for subtypes A, G, B, C, F1, H:
(SEQ ID No. 1)
5′-CTC TGY GTY ACT TTA XXX XXX XXX-3′,
(ii) V2 forward for all subtypes:
(SEQ ID No. 3)
5′-AAA ACT GCT CTT WCA XXX XXX XXX-3′,
(iii) V3 forward for A, B, G subtypes:
(SEQ ID No. 5)
5′-TAV AAA TTA ATT GTA XXX XXX XXX X-3′,
(iv) V3 forward For subtype D:
(SEQ ID No. 7)
5′-TAV CAA TTA ATT GCA XXX XXX XXX X-3′,
(v) V4 forward for all subtypes:
(SEQ ID No. 9)
5′-GAA TTT TTC TAT TGY AAXXX XXX XXX-3′,
(vi) V5 forward for A, B, D, G subtypes:
(SEQ ID No. 11)
5′-ACA AGA GAT GGT GGX XXX XXX X-3′,
(vii) V5 forward For subtype C:
(SEQ ID No. 13)
5′-ACA CGT GAT GGA GGX XXX XXX X-3′,
(viii) gp41(160) Reverse for A, B, D subtypes
ATA GCG GCC GCC TAG TGG TGG TGA TGG TGG TGT AG YAA AGC YCT TTC NAA GCC CTG TC (SEQ ID No. 46) or a derivative thereof lacking the sequence encoding a His-Tag, and/or comprising a sequence encoding a tag,
(ix) gp41(160) Reverse for subtype A, rare variant:
(SEQ ID No. 66)
ATA GCG GCC GCC TAG TGG TGG TGA TGG TGG TGT AGC
AAA GCY CTT TCN GCG CCC TGT C,
(x) gp41(160) Reverse for C subtype:
(SEQ ID No. 67)
ATA GCG GCC GCC TAG TGG TGG TGA TGG TGG TGT WGC
AAA GCT GCT TCA AAG CCC TGT C,
(xi) gp41(160) Reverse for G subtype:
(SEQ ID No. 68)
ATA GCG GCC GCC TAG TGG TGG TGA TGG TGG TGT AGC
AAA GCY CTT TCN AAG CCT TGT C,
and
(xii) gp120 Reverse Const5 for all subtypes:
5′-ATA GCG GCC GCC TAG TGG TGG TGA TGG TGG TGT CTT TTT TCT CTY TSC ACC ACT CTY CT-3′ (SEQ ID No. 52), or a derivative thereof lacking the sequence encoding a His-Tag, and/or comprising a sequence encoding a tag,

13. A composition comprising at least one of following nucleic acids:
(i) Forward gp120 Const1 for A subtype:
5′-AAT TCT AGA CRC TRC AGA AAA CTT GTG GGT YAC-3′ (SEQ ID No. 52), or a derivative thereof lacking the XbaI site, and/or comprising a restriction site at the 5′ end
(ii) Forward gp120 Const1 for B subtype:
5′-AAT TCT AGA CGC TRC AGA AMA ATT GTG GGT CAC-3′(SEQ ID No. 69), or a derivative thereof lacking the XbaI site, and/or comprising a restriction site at the 5′ end,
(iii) Forward gp120 Const1 for C subtype:
5′-AAT TCT AGA CGT RRT GGG RAA CTT GTG GGT CAC-3′ (SEQ ID No. 70), or a derivative thereof lacking the XbaI site, and/or comprising a restriction site at the 5′ end,
(iv) Forward gp120 Const1 for G subtype:
5′-AAT TCT AGA CGC CTC ARA TAA CTT GTG GGT CAC AG-3′(SEQ ID No. 71), or a derivative thereof lacking the XbaI site, and/or comprising a restriction site at the 5′ end,
(v) V1 reverse for all subtypes:
(SEQ ID No. 25)
5′-GCA GTT TTT YAT TTC TYX XXX XXX XXX-3′,
(vi) V2 reverse for subtypes A, D, C:
(SEQ ID No. 28)
5′-AGG TAT TRC AAT TTA TTX XXX XXX X-3′,
(vii) V2 reverse for subtype B:
(SEQ ID No. 29)
5′-CTG AGG TRT TAC AAX XXX XXX X-3′,
(viii) V2 reverse for subtype G:
(SEQ ID No. 30)
5′-AGA CAT TAC AAT TTA TTX XXX XXX X-3′,
(ix) V2 reverse for subtype F1:
(SEQ ID No. 31)
5′-TTG AGG TAT TRC AAX XXX XXX X-3′,
(x) V3 reverse for subtypes A, D, (C):
(SEQ ID No. 40)
5′-AAA GTT TBA TTC CAX XXX XXX XX-3′,
(xi) V3 reverse for subtype B:
(SEQ ID No. 41)
5′-AAA GTG TTR TTC CAX XXX XXX XX-3′,
(xii) V4 reverse for subtype G:
(SEQ ID No. 19)
5′-CAA TTT GTT TTA TYY TAC A XX XXX XXX X-3′,
(xiii) V4 reverse for subtypes A, B, C, D:
(SEQ ID No. 20)
5′-TAA TTT GYT TTA TTY TGC A XX XXX XXX X-3′,
(xiv) V5 reverse for all subtypes:
(SEQ ID No. 15)
5′-TCC TCC TSC AGG TCT GAA XXX XXX XXX X-3′,,
(xv) gp41(160) Reverse for A, B, D subtypes:
ATA GCG GCC GCC TAG TGG TGG TGA TGG TGG TGT AG YAA AGC YCT TTC NAA GCC CTG TC (SEQ ID No. 46), or a derivative thereof lacking the sequence encoding a His-Tag, and/or comprising a sequence encoding a tag,
(xvi) gp41(160) Reverse for subtype A, rare variant:
(SEQ ID No. 66)
ATA GCG GCC GCC TAG TGG TGG TGA TGG TGG TGT AGC
AAA GCY CTT TCN GCG CCC TGT C
(xvii) gp41(160) Reverse for C subtype:
(SEQ ID No. 67)
ATA GCG GCC GCC TAG TGG TGG TGA TGG TGG TGT WGC
AAA GCT GCT TCA AAG CCC TGT C,
and
(xviii) gp41(160) Reverse for G subtype:
(SEQ ID No. 68)
ATA GCG GCC GCC TAG TGG TGG TGA TGG TGG TGT AGC
AAA GCY CTT TCN AAG CCT TGT C.

14. The use of a Severe Combined T-B-Immune Deficient (SCID) mouse engrafted with human immunocompetent cells (Hu-SCID-mouse) as an animal model for the evaluation of the effectiveness of an HIV vaccine.

15. The use of claim 14, wherein the immunocompetent cells are able to develop a human-type immune reaction for HIV.

16. The use of claim 14, wherein the human immunocompetent cells are PBMC, in particular PBMC pre-cultivated in vitro, more particularly PBMC pre-cultivated in vitro for about 1 day to about 6 weeks.

17. The use of claim 14, wherein the human immunocompetent cells are dendritic cells.

18. The use of claim 14, wherein the immunocompetent cells are a mixture of PBMC and dendritic cells.

19. The use of claim 18, wherein the dendritic cells have been obtained by culturing human PBMC in the presence of cytokines capable of inducing the formation of dendritic cells in vitro.

20. The use of any of claims 14 to 19, wherein the human immunocompetent cells are derived from one human donor.

21. The use of any of claims 14 to 20, wherein the mouse has been engrafted with 5-15×10⁶ cells, in particular immunocompetent cells.

22. The use of any of claims 14 to 21, wherein the Hu-SCID mouse is the particular breed of animals deficient in their own endogenous immune system and introduced with human immunocompetent cells or any other human cells.

23. A method for the evaluation of an HIV vaccine, wherein a Hu-SCID-mouse as defined in any of claims 14 to 22 is inoculated with the HIV vaccine and thereafter challenged with HI-virus.

24. The method of claim 23, wherein the HIV vaccine is an HIV-1 envelop peptides/proteins cocktail, preferably wherein the proteins or peptides are of recombinant origin.

25. The method of any of claim 14 or 24, wherein the evaluation of the vaccine is determined by determining the efficacy of the vaccine.

26. The method of claim 25, wherein the efficacy is determined by determining the protection to HIV challenge.

27. The method of any of claims 23 to 26, wherein the Hu-SCID-mouse is inoculated with the HIV vaccine 1 to 4 weeks after the last engrafting of the human immunocompetent cells.

28. The method of any of claims 23 to 27, wherein an infectious dose of 5×10²-10⁴ TCID₅₀ or higher, in particular up to up to 10⁷ TCID₅₀ for challenging of one Hu-SCID animal is used.

29. The method of any of claims 23 to 28, wherein the efficacy of the HIV vaccine is determined by determining the viral load and/or the specificity an/or the intensity of an immune response in a body fluid sample of the mouse.

30. The method of claim 29, wherein the body fluid sample is blood serum.

31. The method of any of claims 23 to 30, wherein the inoculation with a vaccine is performed in the presence of an adjuvant.

32. The method of any of claims 23 to 31, wherein HIV-specific immune response is detectable within the period of several weeks after the last vaccination.

33. The method of any of claims 23 to 32, wherein the evaluation of the vaccine is carried out by means of RT-PCR, Real Time PCR or ELISA, in particular by means of RT-PCR, Real Time PCR and ELISA.

34. The method of any of claims 14 to 33, wherein at least one HIV laboratory strain playback is used to infect and/or expose the Hu-SCID mice with/to HIV.


SEQUENCE LISTING (78 sequences)

SEQ ID NO: 1 - DNA, 24 bp, Artificial Sequence
  V1 forward
ctctgygtyactttannnnnnnnn

SEQ ID NO: 2 - protein, 8 aa, Artificial Sequence
  translated peptide from V1 forward
LCVTLXXX

SEQ ID NO: 3 - DNA, 24 bp, Artificial Sequence
  V2 forward
aaaactgctcttwcannnnnnnnn

SEQ ID NO: 4 - protein, 7 aa, Artificial Sequence
  peptide translated from V2 forward
NCSXXXX

SEQ ID NO: 5 - DNA, 26 bp, Artificial Sequence
  V3 forward
tavaaattaattgtannnnnnnnnnn

SEQ ID NO: 6 - protein, 8 aa, Artificial Sequence
  translated from V3 forward
XINCXXXX

SEQ ID NO: 7 - DNA, 26 bp, Artificial Sequence
  V3 forward for subtype D
tavcaattaattgcannnnnnnnnnn

SEQ ID NO: 8 - protein, 8 aa, Artificial Sequence
  Translated from V3 forward for subtype D
XINCXXXX

SEQ ID NO: 9 - DNA, 26 bp, Artificial Sequence
  V4 forward
gaatttttctattgyaannnnnnnnn

SEQ ID NO: 10 - protein, 8 aa, Artificial Sequence
  Translated from V4 forward
EFFXCXXX

SEQ ID NO: 11 - DNA, 22 bp, Artificial Sequence
  V5 forward
acaagagatggtggnnnnnnnn

SEQ ID NO: 12 - protein, 7 aa, Artificial Sequence
  Translated from V5 forward
TRDGXXX

SEQ ID NO: 13 - DNA, 22 bp, Artificial Sequence
  V5 forward for subtype C
acacgtgatggaggnnnnnnnn

SEQ ID NO: 14 - protein, 7 aa, Artificial Sequence
  Translated from V5 forward for subtype C
TRDGXXX

SEQ ID NO: 15 - DNA, 28 bp, Artificial Sequence
  V5 reverse
tcctcctscaggtctgaannnnnnnnnn

SEQ ID NO: 16 - DNA, 28 bp, Artificial Sequence
  Complementary to V5 reverse
aggaggasgtccagacttnnnnnnnnnn

SEQ ID NO: 17 - DNA, 28 bp, Artificial Sequence
  V5 reverse complement strand
nnnnnnnnnnttcagacctgsaggagga

SEQ ID NO: 18 - protein, 9 aa, Artificial Sequence
  translation of V5 reverse complement strand
XXXFRPXGG

SEQ ID NO: 19 - DNA, 28 bp, Artificial Sequence
  V4 reverse for subtype G
caatttgttttatyytacannnnnnnnn

SEQ ID NO: 20 - DNA, 28 bp, Artificial Sequence
  V4 reverse for subtypes A,B,C,D
taatttgytttattytgcannnnnnnnn

SEQ ID NO: 21 - DNA, 28 bp, Artificial Sequence
  V4 reverse complement strand for subtype G
nnnnnnnnntgtarrataaaacaaattg

SEQ ID NO: 22 - protein, 9 aa, Artificial Sequence
  Translated V4 reverse complement strand for subtype G
XXXCXIKQI

SEQ ID NO: 23 - DNA, 28 bp, Artificial Sequence
  V4 reverse complement strand for subtypes A,B, C,D
nnnnnnnnntgcaraataaarcaaatta

SEQ ID NO: 24 - protein, 9 aa, Artificial Sequence
  Translated V4 reverse complement strand for subtypes A,B,C,D
XXXCXIKQI

SEQ ID NO: 25 - DNA, 27 bp, Artificial Sequence
  V1 reverse
gcagtttttyatttctynnnnnnnnnn

SEQ ID NO: 26 - DNA, 26 bp, Artificial Sequence
  V1 reverse complement strand
nnnnnnnnnragaaatraaaaactgc

SEQ ID NO: 27 - protein, 8 aa, Artificial Sequence
  V1 reverse complement strand
XXXEIKNC

SEQ ID NO: 28 - DNA, 25 bp, Artificial Sequence
  V2 reverse for subtypes A,D,C
aggtattrcaatttattnnnnnnnn

SEQ ID NO: 29 - DNA, 22 bp, Artificial Sequence
  V2 reverse for subtype B
ctgaggtrttacaannnnnnnn

SEQ ID NO: 30 - DNA, 25 bp, Artificial Sequence
  V2 reverse for subtype G
agacattacaatttattnnnnnnnn

SEQ ID NO: 31 - DNA, 22 bp, Artificial Sequence
  V2 reverse for subtype F1
ttgaggtattrcaannnnnnnn

SEQ ID NO: 32 - DNA, 25 bp, Artificial Sequence
  V2 reverse complement strand for subtypes A,D,C
nnnnnnnnaataaattgyaatacct

SEQ ID NO: 33 - protein, 8 aa, Artificial Sequence
  Translated V2 reverse complement strand for subtypes A,D,C
XXXINCNT

SEQ ID NO: 34 - DNA, 23 bp, Artificial Sequence
  V2 reverse complement strand for subtype B
nnnnnnnnnttgtaayacctcag

SEQ ID NO: 35 - protein, 7 aa, Artificial Sequence
  Translated V2 reverse complement strand for subtype B
XXXCNTS

SEQ ID NO: 36 - DNA, 25 bp, Artificial Sequence
  V2 reverse complement strand for G subtype
nnnnnnnnaataaattgtaatgtct

SEQ ID NO: 37 - protein, 8 aa, Artificial Sequence
  Translated V2 reverse complement strand for G subtype
XXXINCNV

SEQ ID NO: 38 - DNA, 23 bp, Artificial Sequence
  V2 reverse complement strand for subtype F1
nnnnnnnnnttgyaatacctcaa

SEQ ID NO: 39 - protein, 7 aa, Artificial Sequence
  Translated V2 reverse complement strand for subtype F1
XXXCNTS

SEQ ID NO: 40 - DNA, 23 bp, Artificial Sequence
  V3 reverse for subtypes A, D, (C)
aaagtttbattccannnnnnnnn

SEQ ID NO: 41 - DNA, 23 bp, Artificial Sequence
  V3 reverse for subtype B
aaagtgttrttccannnnnnnnn

SEQ ID NO: 42 - DNA, 23 bp, Artificial Sequence
  V3 reverse complement strand for subtypes A, D, (C)
nnnnnnnnntggaatvaaacttt

SEQ ID NO: 43 - protein, 7 aa, Artificial Sequence
  Translated V3 reverse complement strand for subtypes A, D, (C)
XXXWNXT

SEQ ID NO: 44 - DNA, 23 bp, Artificial Sequence
  V3 reverse complement strand for subtype B
nnnnnnnnntggaayaacacttt

SEQ ID NO: 45 - protein, 7 aa, Artificial Sequence
  Translated V3 reverse complement strand for subtype B
XXXWNNT

SEQ ID NO: 46 - DNA, 58 bp, Artificial Sequence
  gp41(160) Reverse for A,B,D subtypes
atagcggccgcctagtggtggtgatggtggtgtagyaaagcyctttcnaagccctgtc

SEQ ID NO: 47 - DNA, 58 bp, Artificial Sequence
  gp41(160) Reverse complement strand for A,B,D subtypes
gacagggcttngaaagrgctttrctacaccaccatcaccaccactaggcggccgctat

SEQ ID NO: 48 - protein, 14 aa, Artificial Sequence
  Translated gp41(160) Reverse complement strand for A,B,D subtypes
QGXERALLHHHHHH

SEQ ID NO: 49 - DNA, 59 bp, Artificial Sequence
  gp120 Reverse Const5 for all subtypes
atagcggccgcctagtggtggtgatggtggtgtcttttttctctytscaccactctyct

SEQ ID NO: 50 - DNA, 59 bp, Artificial Sequence
  gp120 Reverse complement strand Const5 for all subtypes
agragagtggtgsaragagaaaaaagacaccaccatcaccaccactaggcggccgctat

SEQ ID NO: 51 - protein, 15 aa, Artificial Sequence
  Translated gp120 Reverse complement strand Const5 for all subtypes
RRVVXREKRHHHHHH

SEQ ID NO: 52 - DNA, 33 bp, Artificial Sequence
  Forward gp120 Const1 for A subtype
aattctagacrctrcagaaaacttgtgggtyac

SEQ ID NO: 53 - protein, 18 aa, Artificial Sequence
  Translated Forward gp120 Const1 for A subtype
LDXXENLWVLDXXENLWV

SEQ ID NO: 54 - protein, 856 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 55 - protein, 511 aa, Human immunodeficiency virus
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 56 - protein, 345 aa, Human immunodeficiency virus
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 57 - protein, 842 aa, Human immunodeficiency virus
MRVRGMQRNWQTLGNWGILFLGILIICSNADKLWVTVYYGVPVWKEATPTLFCASDAKAYEKEVHNVWATHACVPTDPNPQEVEMENVTENFNMWKNNMVEQMHTDIISLWDESLKPCVELTPLCVTLNCTDYKGTNSTNNATSTVVSPAEIKNCSFNITTEIKDKKKKESALFYRLDVLPLNGEGNNSSTEYRLINCNTSTITQTCPKVTFEPIPIHYCAPAGFAILKCKDKRFNGTGPCKNVSTVQCTHGIKPVVSTQLLLNGSLAEEEIIIRSENITDNTKNIIVQLNETVQINCTRPNNNTRKSIHMGPGKAFYTTGDIIGDIRQAHCNISGEKWNMTLSRVKEKLKEHFKNGTITFKPPNPGGDPEILTHMFNCAGEFFYCNTTKLFNETGENGTITLPCRIKQIINMWQKVGKAIYAPPIAGSINCSSNITGMILTRDGGNNTHNETFRPGGGDMRDNWRSELYKYKVVQIEPLGIAPTRARRRVVQREKRAVGLGAVFFGFLGAAGSTMGAASITLTVQARQLLSGIVQQQSNLLRAIEAQQHLLQLTVWGIKQLRARILAVERYLKDQQLLGIWGCSGKLICTTNVPWNSSWSNKSWEEIWNNMTWMEWEKEIGNYSDTIYKLIEESQTQQEKNEQDLLALDKWASLWNWFDITKWLWYIKIFIMIIGGLIGLRIAFAVLSVVNRVRQGYSPLSFQTLIPTSRGADRPEGIEEEGGEQDKNRSVRLVSGFLALAWDDLRNLCLFSYRQLRNLILIVTRILERGLRGGWEALKYLWNLVQYWSQELKNSAISLLNTTAIAVAGGTDRIIEIGQRAFRALLHIPRRIRQGLERALL

SEQ ID NO: 58 - protein, 497 aa, Human immunodeficiency virus
MRVRGMQRNWQTLGNWGILFLGILIICSNADKLWVTVYYGVPVWKEATPTLFCASDAKAYEKEVHNVWATHACVPTDPNPQEVEMENVTENFNMWKNNMVEQMHTDIISLWDESLKPCVELTPLCVTLNCTDYKGTNSTNNATSTVVSPAEIKNCSFNITTEIKDKKKKESALFYRLDVLPLNGEGNNSSTEYRLINCNTSTITQTCPKVTFEPIPIHYCAPAGFAILKCKDKRFNGTGPCKNVSTVQCTHGIKPVVSTQLLLNGSLAEEEIIIRSENITDNTKNIIVQLNETVQINCTRPNNNTRKSIHMGPGKAFYTTGDIIGDIRQAHCNISGEKWNMTLSRVKEKLKEHFKNGTITFKPPNPGGDPEILTHMFNCAGEFFYCNTTKLFNETGENGTITLPCRIKQIINMWQKVGKAIYAPPIAGSINCSSNITGMILTRDGGNNTHNETFRPGGGDMRDNWRSELYKYKVVQIEPLGIAPTRARRRVVQREKR

SEQ ID NO: 59 - protein, 345 aa, Human immunodeficiency virus
AVGLGAVFFGFLGAAGSTMGAASITLTVQARQLLSGIVQQQSNLLRAIEAQQHLLQLTVWGIKQLRARILAVERYLKDQQLLGIWGCSGKLICTTNVPWNSSWSNKSWEEIWNNMTWMEWEKEIGNYSDTIYKLIEESQTQQEKNEQDLLALDKWASLWNWFDITKWLWYIKIFIMIIGGLIGLRIAFAVLSVVNRVRQGYSPLSFQTLIPTSRGADRPEGIEEEGGEQDKNRSVRLVSGFLALAWDDLRNLCLFSYRQLRNLILIVTRILERGLRGGWEALKYLWNLVQYWSQELKNSAISLLNTTAIAVAGGTDRIIEIGQRAFRALLHIPRRIRQGLERALL

SEQ ID NO: 60 - protein, 825 aa, Human immunodeficiency virus
  MISC_FEATURE (18)..(18): any natural amino acid
MKAKGMQRNYQHLWRWGXMLFWXIIMCKAAENLWVTVYYGVPVWRDAETTLFCASDAKAYDKEVHNVWATHACVPTDPNPQEIALENVTEKFDMWKNNMVEQMQTDIISLWDQSLKPCVKLTPLCVTLNCAEPNSTSSNNNSVNSNSSDSVFEEMKNCTFNMTTELRDKRKTVHSLFYKLDIVSTGSNGSGQYRLINCNTSAMTQACPKVTFEPIPIHYCAPAGFAILKCKDTNFTGTGPCKNVSTVQCTHGIKPVVSTQLLLNGSLAEKEVMXRSENITDNGKXIIVQLTEPVNITCIRPGNNTRTSIRIGPGQTFYATXDVIGDIRKAYCXVSRAAWXSTLQKISTQLRKYFNNKTIXFKNSSGGDLEVTTHSFNCGGEFFYCNTTDLFNSTWDGXGTXTXITXANGTITLPCRIKQIINMWQRVGQAMYAPPIKGSIRCESNITGLXLTRDGGGGTNXXXETFRPIGGNMRDNWRSELYKYKVVKIEPIGVAPTRAKRRVVEREKRAIGLGAAFLGFLGAAGSTMGAASMTLTVQARQLLSGIVQQQSNLLRAIEAQQHLLKLTVWGIKQLQARVLAVERYLKDQQLLGIWGCSGKLICTTNVPWNSSWSNKSQSEIWDNMTWMQWDKEVINYTXIIYDLIEKSQNQQEKNEQDLLALDKWASLWXWFDISNWLWYIKIFIIIVGSLIGLRIXFAVLYIINRARQGYSPLSLQTLTPHPEGPDRPGRIKEEGGEQGRDRSIRLVSGFLALAWDDLRSLCLFSYHRLRDFIXIAARTVELLGRSSLKGLRLGWEGLKYLGNLLGYWGQELKSSAINLIDTIAI

SEQ ID NO: 61 - protein, 509 aa, Human immunodeficiency virus
  MISC_FEATURE (18)..(18): any natural amino acid
MKAKGMQRNYQHLWRWGXMLFWXIIMCKAAENLWVTVYYGVPVWRDAETTLFCASDAKAYDKEVHNVWATHACVPTDPNPQEIALENVTEKFDMWKNNMVEQMQTDIISLWDQSLKPCVKLTPLCVTLNCAEPNSTSSNNNSVNSNSSDSVFEEMKNCTFNMTTELRDKRKTVHSLFYKLDIVSTGSNGSGQYRLINCNTSAMTQACPKVTFEPIPIHYCAPAGFAILKCKDTNFTGTGPCKNVSTVQCTHGIKPVVSTQLLLNGSLAEKEVMXRSENITDNGKXIIVQLTEPVNITCIRPGNNTRTSIRIGPGQTFYATXDVIGDIRKAYCXVSRAAWXSTLQKISTQLRKYFNNKTIXFKNSSGGDLEVTTHSFNCGGEFFYCNTTDLFNSTWDGXGTXTXITXANGTITLPCRIKQIINMWQRVGQAMYAPPIKGSIRCESNITGLXLTRDGGGGTNXXXETFRPIGGNMRDNWRSELYKYKVVKIEPIGVAPTRAKRRVVEREKR

SEQ ID NO: 62 - protein, 352 aa, Human immunodeficiency virus
  MISC_FEATURE (129)..(129): any natural amino acid
AIGLGAAFLGFLGAAGSTMGAASMTLTVQARQLLSGIVQQQSNLLRAIEAQQHLLKLTVWGIKQLQARVLAVERYLKDQQLLGIWGCSGKLICTTNVPWNSSWSNKSQSEIWDNMTWMQWDKEVINYTXIIYDLIEKSQNQQEKNEQDLLALDKWASLWXWFDISNWLWYIKIFIIIVGSLIGLRIXFAVLYIINRARQGYSPLSLQTLTPHPEGPDRPGRIKEEGGEQGRDRSIRLVSGFLALAWDDLRSLCLFSYHRLRDFIXIAARTVELLGRSSLKGLRLGWEGLKYLGNLLGYWGQELKSSAINLIDTIAIAVARWTDXVIEIGQRLCRAIRNIPRRIRQGXEKALQ

SEQ ID NO: 63 - protein, 878 aa, Human immunodeficiency virus
MRVRGIPRNWQQWWIWIILGFWMLLICNVGGNSWVTIYYGVPVWREAKTTLFCASDAKAHETEVHNVWATHACVPTDPNPQEIELENVTENFNMWKNDMVDQMHEDIISLWDQSLKPCVKLTPLCVTLNCTNATRPVTRTNTTATGTNNTVTNCSGSASTNNTCMENIEGMKNCSFNITTELRDKKKKEYALFYRLDIVPLNETRDNSSYRLINCNTSTITQACPKVSFDPIPIHYCAPAGYAILKCNNKTFSGTGPCNNVSTVQCTHGIMPVVSTQLLLNGSLAEEMVIIRSDNMTNAATTIIVHLKDPVEIVCTRPNNTTRREVGIGPGQTFYTTGQIIGDIRQAHCNITGKEWNKTLRQVGAELEKHFPNKTIQFKPHSGGDLEITTHSFTCSGEFFYCNTSKLFNISMSNLTYNNTDNTDNPTQITLPCRIKQIINMWQEVGRAIYAPPIAGNITCKSNITGLLLTWDGGSGENNTETFRPGGGDMRDNWRSELYKYKVVEIKPLGIAPTEAKRRVVEREKRAIGIGAVFLGFLGAAGSTMGAASITLTVQARQLLSGIVQQQSNLLRAIEAQQHLLQLTVWGIKQLQARVLAIERYLKDQQLLGIWGCSGKLICTTSVPWNSSWSNKTLGEIWNNMTWMEWDKEISNYTHTIYQLLEESQIQQEQNEKELLALDSWKNLWNWFDISNWLWYIKIFIMIVGGLIGLRIVFAVLSIVNRVRQGYSPLSFQTLTPSPREPDRLRGIEEEGGEQDKGRSIRLVQGFLALAWDDLRSLCLFSYHRLRDFISIAARVVEVLGHSSLRGLQRGWEALKYLKSLVQYWGLELKKSAVSLLDTLAIAVGEGTDRIIELIQGICRAIRNIPRRIRQGFEAALL

SEQ ID NO: 64 - protein, 526 aa, Human immunodeficiency virus
MRVRGIPRNWQQWWIWIILGFWMLLICNVGGNSWVTIYYGVPVWREAKTTLFCASDAKAHETEVHNVWATHACVPTDPNPQEIELENVTENFNMWKNDMVDQMHEDIISLWDQSLKPCVKLTPLCVTLNCTNATRPVTRTNTTATGTNNTVTNCSGSASTNNTCMENIEGMKNCSFNITTELRDKKKKEYALFYRLDIVPLNETRDNSSYRLINCNTSTITQACPKVSFDPIPIHYCAPAGYAILKCNNKTFSGTGPCNNVSTVQCTHGIMPVVSTQLLLNGSLAEEMVIIRSDNMTNAATTIIVHLKDPVEIVCTRPNNTTRREVGIGPGQTFYTTGQIIGDIRQAHCNITGKEWNKTLRQVGAELEKHFPNKTIQFKPHSGGDLEITTHSFTCSGEFFYCNTSKLFNISMSNLTYNNTDNTDNPTQITLPCRIKQIINMWQEVGRAIYAPPIAGNITCKSNITGLLLTWDGGSGENNTETFRPGGGDMRDNWRSELYKYKVVEIKPLGIAPTEAKRRVVEREKR

SEQ ID NO: 65 - protein, 352 aa, Human immunodeficiency virus
AIGIGAVFLGFLGAAGSTMGAASITLTVQARQLLSGIVQQQSNLLRAIEAQQHLLQLTVWGIKQLQARVLAIERYLKDQQLLGIWGCSGKLICTTSVPWNSSWSNKTLGEIWNNMTWMEWDKEISNYTHTIYQLLEESQIQQEQNEKELLALDSWKNLWNWFDISNWLWYIKIFIMIVGGLIGLRIVFAVLSIVNRVRQGYSPLSFQTLTPSPREPDRLRGIEEEGGEQDKGRSIRLVQGFLALAWDDLRSLCLFSYHRLRDFISIAARVVEVLGHSSLRGLQRGWEALKYLKSLVQYWGLELKKSAVSLLDTLAIAVGEGTDRIIELIQGICRAIRNIPRRIRQGFEAALL

SEQ ID NO: 66 - DNA, 58 bp, Artificial Sequence
  gp41(160) Reverse for subtype A (rare variant)
atagcggccgcctagtggtggtgatggtggtgtagcaaagcyctttcngcgccctgtc

SEQ ID NO: 67 - DNA, 58 bp, Artificial Sequence
  gp41(160) Reverse for C subtype
atagcggccgcctagtggtggtgatggtggtgtwgcaaagctgcttcaaagccctgtc

SEQ ID NO: 68 - DNA, 58 bp, Artificial Sequence
  gp41(160) Reverse for G subtype
atagcggccgcctagtggtggtgatggtggtgtagcaaagcyctttcnaagccttgtc

SEQ ID NO: 69 - DNA, 33 bp, Artificial Sequence
  Forward gp120 Const1 for B subtype
aattctagacgctrcagaamaattgtgggtcac

SEQ ID NO: 70 - DNA, 33 bp, Artificial Sequence
  Forward gp120 Const1 for C subtype
aattctagacgtrrtgggraacttgtgggtcac

SEQ ID NO: 71 - DNA, 35 bp, Artificial Sequence
  Forward gp120 Const1 for G subtype
aattctagacgcctcarataacttgtgggtcacag

SEQ ID NO: 72 - protein, 5 aa, Artificial Sequence
  minimal translation of V1 forward for subtypes A,G B,C,F1,H
LCVTL

SEQ ID NO: 73 - protein, 4 aa, Artificial Sequence
  minimal translation of V2 forward for all subtypes
NCSX

SEQ ID NO: 74 - protein, 4 aa, Artificial Sequence
  minimal translation of V3 forward for A, B, G subtypes
XINC

SEQ ID NO: 75 - protein, 4 aa, Artificial Sequence
  minimal translation of V3 forward for subtype D
XINC

SEQ ID NO: 76 - protein, 5 aa, Artificial Sequence
  minimal translation of V4 forward for all subtypes
EFFYC

SEQ ID NO: 77 - protein, 4 aa, Artificial Sequence
  minimal translation of V5 forward
TRDG

SEQ ID NO: 78 - protein, 11 aa, Artificial Sequence
  minimal translation of Forward gp120 Const1 for A subtype
LDXXENLWVLD